(19) United States
(12) Patent Application Publication
(10) Pub. No.: US 2009/0042856 A1
(43) Pub. Date: Feb. 12, 2009

(54) PYRIDAZINONE DERIVATIVES USED FOR THE TREATMENT OF PAIN

(75) Inventors: Hitoshi Yamazaki, Tokyo (JP); Chiyoshi Kasahara, Tokyo (JP); Hirokazu Kubota, Tokyo (JP); Toru Kontani, Tokyo (JP); Toru Asano, Tokyo (JP); Hidekazu Mizuhara, Tokyo (JP); Masaharu Yokomoto, Hiroshima (JP); Keiji Misumi, Hiroshima (JP); Tomohiko Kinoshita, Hiroshima (JP)

Correspondence Address: OBLON, SPIVAK, MCCLELLAND MAIER & NEUSTADT, P.C., 1940 DUKE STREET, ALEXANDRIA, VA, 22314, US

(73) Assignees: Astellas Pharma Inc, Chuo-ku (JP); Wakunaga Pharmaceutical Co., Ltd., Osaka (JP)

(21) Appl. No.: 12/063,766

(22) PCT Filed: Aug. 31, 2006
(86) PCT No.: PCT/JP2006/317691

§ 371 Date: Jun. 4, 2008

Related U.S. Application Data
(60) Provisional application No. 60/712,825, filed on Sep. 1, 2005.

(51) Int. Cl. A61K 31/519; A61P 25/00; C07D 487/04; C07D 487/10; C07D 491/20; C07D 471/20; A61K 31/527; C07D 498/04; A61K 31/553; A61K 31/5383; A61K 31/5377; A61K 31/5025

(52) U.S. Cl. 514/210.21; 544/238; 544/114; 544/117; 544/231; 544/105; 540/552; 514/211.1; 514/230.5; 514/233.2; 514/249; 514/259.3; 514/267

(57) ABSTRACT

A pyridazinone derivative compound shown by the following formula (I): wherein R₁ is selected from hydrogen, etc.; R₂ is selected from substituted or unsubstituted aryl, etc.; R₃ is hydrogen, etc.; p is 0, 1 or 2; R₄ and R₅, are each hydrogen, etc.; R₆ and R₇, are taken together to form a group of the formula: wherein R₈ is hydrogen; X is selected from oxygen, etc; R₁₀ is selected from hydrogen, etc.; R₁₁ is selected from hydrogen, etc.; R₁₂ is selected from hydrogen, etc.; R₁₃ is selected from hydrogen, etc.; R₁₄ is selected from hydrogen, etc.; m and n are each 0, 1, or 2, or a pharmaceutically acceptable salt thereof, which is useful as a medicament.

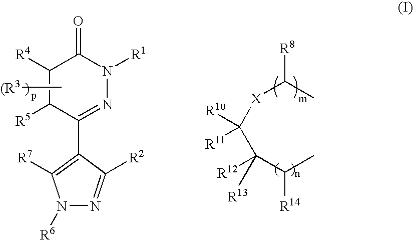

### FIELD OF THE INVENTION

The present invention relates to a pyridazinone derivative compound and a salt thereof, which are useful for medicaments.

### BACKGROUND ART

Rheumatoid arthritis (RA) is a systemic inflammatory disease which causes mainly in the arthrosynovia. Today Methotrexate (MTX) is used generally as a disease-modified anti-rheumatic drugs (DMARD), but the efficacy for inflammatory responses or arthritis mutilans is not enough. On the other hand, the biologics, which targeted cytokines (TNF, IL-1, IL-6), has been revealed recently its efficacy for RA, and it has been proved the importance of these cytokines in the manifestation of RA. In particular, the monoclonal TNF antibody Remicade and soluble TNF receptor fusion protein Enbrel, which inhibit the TNF function, are worthy of note because of the unprecedented efficacy not only for inflammatory response but for arthritis mutilans.

Though the fact above suggests that the importance of the treatment for RA in future, these biologics have fundamental drawbacks related to patient cost, efficacy of production, limitation of administration to hypodermal or intravenous injection, and so on. So, the anti-RA drugs in the next generation are expected to overcome these problems, that is to be an orally small-molecule drug, which blocks or modulates selectively the function of these cytokines. In particular p38α mitogen activated protein kinase (p38α MAPK) belongs to intracellular phosphorylation kinase participating in production and/or functional expression of the cytokine (TNF, IL-1, IL-6), and it is reported that p38α MAPK is activated in the arthrosynovia of RA patients thereby cytokines are produced excessively, so that p38α MAPK has been attracted as a target of anti-RA drug.

These anti-inflammatory agents or compounds having cytokine inhibitory activity have been described (WO98/22457, WO00/41698, WO00/43384, WO01/22965, WO 02/07772, WO02/58695, WO03/041644, etc.) but a pyridazinone derivative having these activity is novel as far as we know.

### SUMMARY OF THE INVENTION

The present invention relates to a pyridazinone derivative compound and a pharmaceutically acceptable salt thereof, which are useful as medicaments; a pharmaceutical composition comprising, as an active ingredient, said pyridazinone derivative compound or a pharmaceutically acceptable salt thereof; a use of said pyridazinone derivative compound or a pharmaceutically acceptable salt thereof as a medicament; and a method for using said pyridazinone derivative compound or a pharmaceutically acceptable salt thereof for therapeutic purposes, which comprises administering said pyridazinone derivative compound or a pharmaceutically acceptable salt thereof to a mammal.

The pyridazinone derivative compound and a salt thereof are inhibitors of cytokines' production or their transduction, and through inhibiting the p38α MAPK they possess pharmacological actions such as analgesic action, anti-inflammatory, anti arthritis mutilans action, or the like.

They are useful as an analgesic, in particular anti-RA agent, drug for pain and other conditions associated with inflammation, drug for Crohn's disease, drug for inflammatory bowel disease, drug for psoriasis, or the like.

The pyridazinone derivative compound or a salt thereof of the present invention is a pyridazinone derivative compound shown by the following formula (I) (hereinafter also simply referred to as compound (I)):

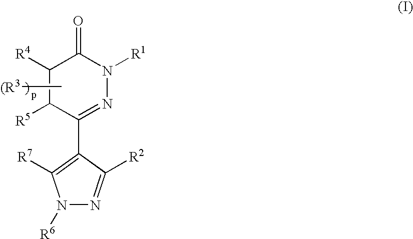

wherein
R¹ is selected from the group consisting of hydrogen, substituted or unsubstituted lower alkyl and substituted or unsubstituted aryl;
R² is selected from the group consisting of substituted or unsubstituted aryl and substituted or unsubstituted heteroaryl;
R³ is lower alkyl;
p is 0, 1 or 2; and
R⁴ and R⁵ are each hydrogen or taken together to form a bond;
R⁶ and R⁷ are taken together to form a group of the formula:

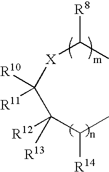

wherein
R⁸ is hydrogen,
X is oxygen or N—R⁹, in which R⁹ is hydrogen, substituted or unsubstituted lower alkanoyl or substituted or unsubstituted lower alkyl; or
R⁸ and R⁹ may be taken together to form a bond;
m and n are each 0, 1 or 2;
R¹⁰ and R¹² are each selected from the group consisting of hydrogen, halogen, hydroxy, formyl, cyano, substituted or unsubstituted lower alkyl, substituted or unsubstituted amino, substituted or unsubstituted lower alkoxy, saturated cyclic amino, substituted or unsubstituted carbamoyl, carboxy, substituted or unsubstituted lower alkoxycarbonyl and substituted or unsubstituted acyloxy;
R¹¹, R¹³ and R¹⁴ are each selected from the group consisting of hydrogen, halogen, substituted or unsubstituted lower alkyl, carboxy and substituted or unsubstituted lower alkoxycarbonyl;
R¹⁰ and R¹¹ or R¹² and R¹³ may be taken together to form oxo, hydroxyimino, substituted or unsubstituted lower alkylene in which one or more carbon(s) may be replaced by hetero atom(s), or substituted or unsubstituted lower alkylidene;
R⁹ and R¹⁰ may be taken together to form lower alkylene or a bond;
R¹¹ and R¹² or R¹³ and R¹⁴ may be taken together to form a bond;
   provided that when n=1 and R¹⁰, R¹¹, R¹², R¹³ and R¹⁴ are simultaneously hydrogen, R⁹ is substituted or unsubstituted lower alkyl or substituted or unsubstituted lower alkanoyl,
   or a pharmaceutically acceptable salt thereof.

One of the preferred embodiments of the present invention can be represented by the compound (I), wherein
R¹ is hydrogen or substituted or unsubstituted aryl;
R² is substituted or unsubstituted aryl;
p is 0;

R¹ and R⁵ are each hydrogen or taken together to form a bond; and
R⁶ and R⁷ are taken together to form a group of the formula:

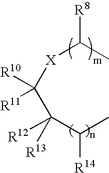

wherein
R⁸ is hydrogen;
X is oxygen or N—R⁹, in which R⁹ is hydrogen, substituted or unsubstituted lower alkanoyl or substituted or unsubstituted lower alkyl; or
R⁸ and R⁹ may be taken together to form a bond;
m and n are each 0, 1 or 2;
R¹⁰ and R¹² are each selected from the group consisting of hydrogen, halogen, hydroxy, formyl, cyano, substituted or unsubstituted lower alkyl, substituted or unsubstituted amino, substituted or unsubstituted lower alkoxy, saturated cyclic amino, substituted or unsubstituted carbamoyl, carboxy, substituted or unsubstituted lower alkoxycarbonyl and substituted or unsubstituted acyloxy;
R¹¹, R¹³ and R¹⁴ are each selected from the group consisting of hydrogen, halogen and substituted or unsubstituted lower alkyl;
R¹⁰ and R¹¹ or R¹² and R¹³ may be taken together to form oxo, hydroxyimino, substituted or unsubstituted lower alkylene in which one or more carbon(s) may be replaced by hetero atom(s), or substituted or unsubstituted lower alkylidene;
R⁹ and R¹⁰ may be taken together to form lower alkylene or a bond;
R¹¹ and R¹² or R¹³ and R¹⁴ may be taken together to form a bond,
   provided that when n=1 and R¹⁰, R¹¹, R¹²R¹³ and R¹⁴ are simultaneously hydrogen, R⁹ is substituted or unsubstituted lower alkyl or substituted or unsubstituted lower alkanoyl,
   or a pharmaceutically acceptable salt thereof.

Another one of the preferred embodiments of the present invention can be represented by the compound (I), wherein
R¹ is hydrogen or (C₆₋₁₄)aryl optionally substituted by (C₁₋₆)alkyl or (C₁₋₆) alkylaminosulfonyl;
R² is (C₆₋₁₄)aryl optionally substituted by 1 to 3 substituent(s) selected from halogen, (C₁₋₆)alkyl and (C₁₋₆)alkoxy;
p is 0;
R⁴ and R⁵ are each hydrogen or taken together to form a bond; and
R⁶ and R⁷ are taken together to form a group of the formula:

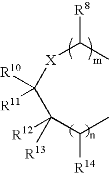

wherein
R⁸ is hydrogen;
X is oxygen or N—R⁹, in which R⁹ is hydrogen, (C₁₋₆)alkyl optionally substituted by carboxy, hydroxy, (C₁₋₆)alkoxycarbonyl, morpholino, morpholinocarbonyl or (C₁₋₆)alkylsulfonyloxy, or (C₂₋₇) alkanoyl; or
R⁸ and R⁹ are taken together to form a bond;
m and n are each 0, 1 or 2;
R¹⁰ is hydrogen, or (C₁₋₆)alkyl optionally substituted by (C₆₋₁₄) aryl(C₁₋₆) alkoxy, di(C₆₋₁₄) aryl(C₁₋₆)alkylsilyloxy or hydroxy;
R¹¹ is hydrogen or (C₁₋₆)alkyl;
R¹² is selected from the group consisting of hydrogen;
   halogen;
   hydroxy;
   carboxy;
   formyl;
   cyano;
   (C₁₋₆)alkyl optionally substituted by hydroxy, hydroxyimino, halogen, (C₁₋₆)alkoxy, (C₁₋₇)alkanoyloxy, amino, mono- or di-(C₁₋₆)alkylamino (wherein one or both of said (C₁₋₆)alkyl is (are) optionally substituted by hydroxy, (C₁₋₆)alkoxy, (C₆₋₁₄)aryl or (C₃₋₆) cycloalkyl-carbonyl), (C₁₋₆)alkylureido, morpholino, or 4- to 6-membered cyclic amino optionally substituted by hydroxy, (C₁₋₆)alkyl or di(C₁₋₆)alkylamino;
   mono- or di-(C₁₋₆)alkylamino;
   4- to 6-membered cyclic amino;
   C₁₋₆ alkoxy optionally substituted by (C₆₋₁₄) aryl;
   carbamoyl optionally substituted by (C₃₋₆) cycloalkyl or hydroxy(C₁₋₆)alkyl;
   (C₁₋₆)alkoxy-carbonyl; and
   (C₁₋₆)alkoxy-carbonyloxy;
R¹³ is hydrogen, or (C₁₋₆)alkyl optionally substituted by hydroxy or (C₁₋₇)alkanoyloxy;
R¹⁴ is hydrogen;
R¹⁰ and R¹¹ may be taken together to form (C₂₋₆)alkylene in which one or more carbon atom(s) may be replaced with heteroatom(s), which is optionally substituted by (C₆₋₁₄)aryl(C₁₋₆)alkoxycarbonyl or (C₁₋₇)alkanoyl;
R¹² and R¹³ may be taken together to form C₂₋₆ alkylene in which one or more carbon atom(s) may be replaced with heteroatom(s) which is optionally substituted by (C₁₋₆)alkyl optionally substituted by hydroxy, or (C₁₋₇)alkanoyl optionally substituted by C₁₋₆ alkoxy;
   (C₁₋₆)alkylidene optionally substituted by hydroxy;
   oxo; or
   hydroxyimino;
   R⁹ and R¹⁰ may be taken together to form (C₂₋₆)alkylene or a bond;
   R¹¹ and R¹³ may be taken together to form a bond; or
   R¹³ and R¹⁴ may be taken together to form a bond;
      provided that when n=1 and R¹⁰, R¹¹, R¹², R¹³ and R¹⁴ are simultaneously hydrogen, R⁹ is substituted or unsubstituted lower alkyl or substituted or unsubstituted lower alkanoyl;
      or a pharmaceutically acceptable salt thereof.

### DETAILED DESCRIPTION OF THE INVENTION

The compound (I) and a salt thereof of the present invention can be prepared by the following processes.

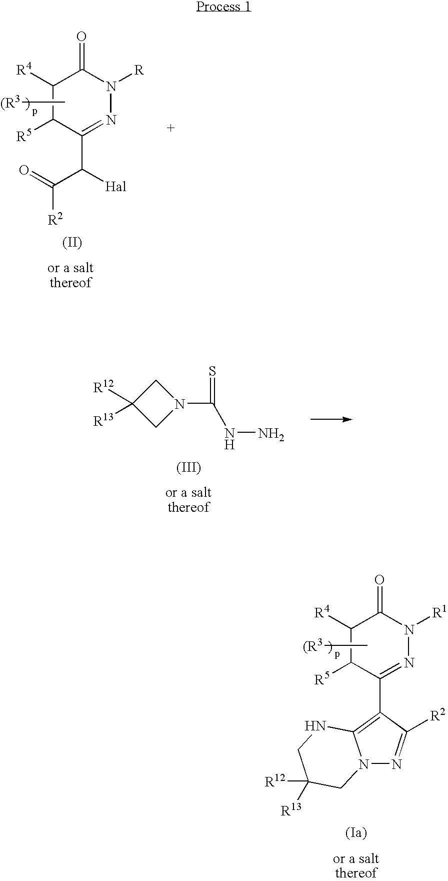

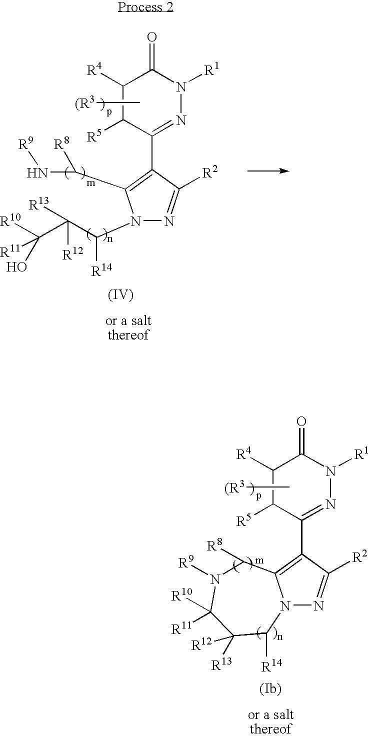

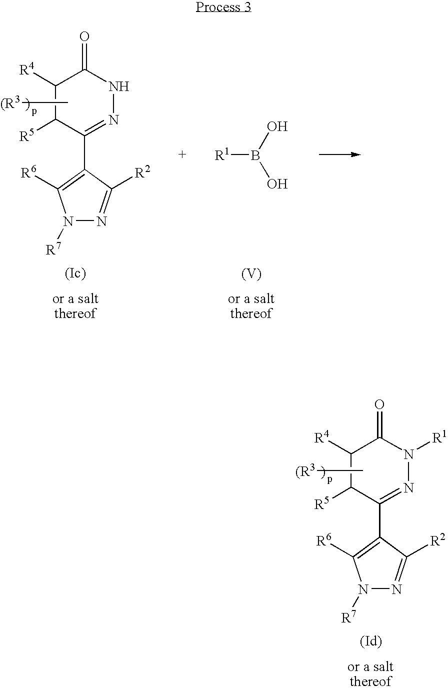

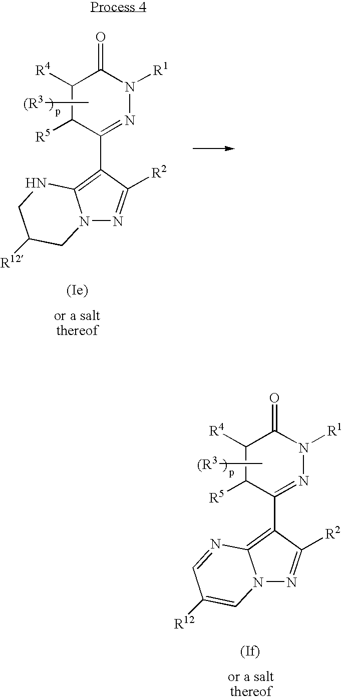

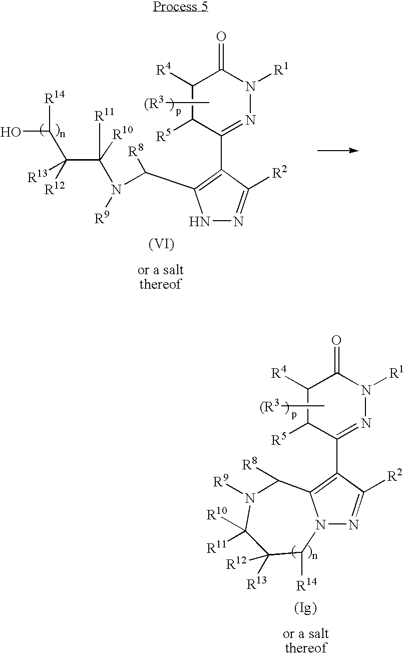

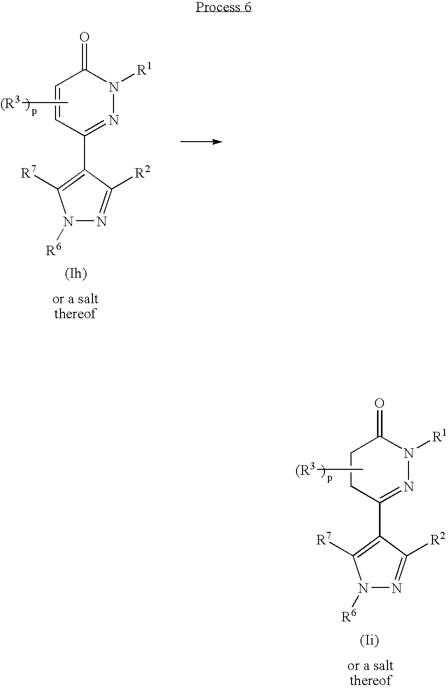

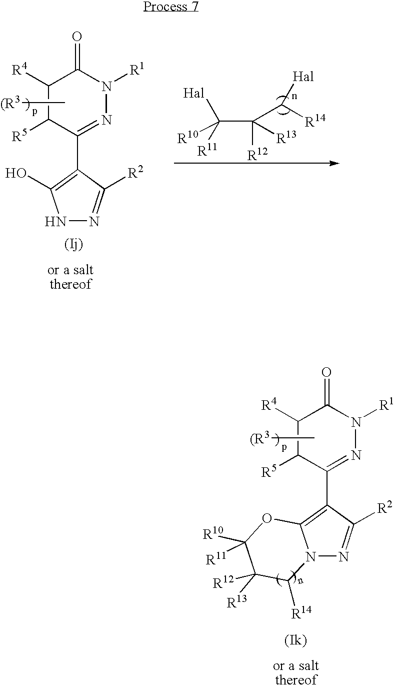

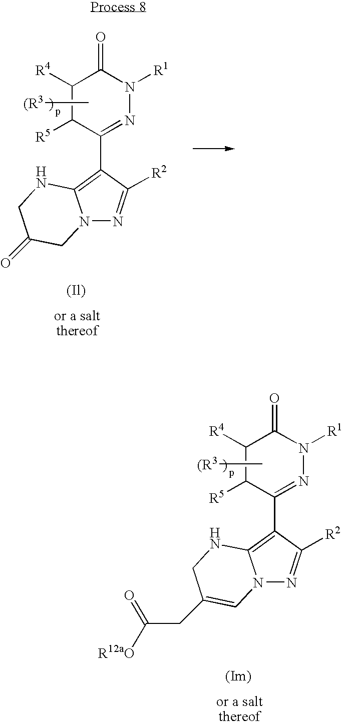

In the formulas in the above-mentioned Processes, R¹, R², R³, R⁴, R⁵, R⁶, R⁷, R⁸, R⁹, R¹⁰, R¹¹, R¹², R¹³, R¹⁴, m, n and p are as defined above;

R^{12′} is similar to R¹²;

R^{12a} is (C₁₋₆)alkyl (e.g., methyl, ethyl, propyl, no butyl, tert-butyl, pentyl, hexyl, etc.); and

Hal is a halogen atom (e.g., bromo, chloro, iodo).

For example, Process 1 is exemplified by Example 1 or the like; Process 2 is exemplified by Example 6 or the like; Process 3 is exemplified by Example 15 or the like; Process 4 is exemplified by Example 2 or the like; Process 5 is exemplified by Example 7 and Example 60, successively or the like; Process 6 is exemplified by Example 55 or the like; Process 7 is exemplified by Example 125 or the like; and Process 8 is exemplified by Example 131 or the like.

In addition to the processes as mentioned above, the compound (I) and a salt thereof can be prepared, for example, according to the procedures as illustrated in Examples in the present specification or in a manner similar thereto.

The starting compounds can be prepared, for example, according to the procedures as illustrated in Preparations in the present specification or in a manner similar thereto.

The compound (I) and a salt thereof can be prepared according to the methods as shown in Preparations or Examples, or in a manner similar thereto.

It is to be noted that all solvated forms of the compound (I) (e.g. hydrates, ethanolates, etc.), all stereoisomers of the compound (I) (e.g., enantiomers, diastereomers, racemic compounds, etc.) and crystal forms of the compound (I) are also included within the scope of the present invention.

It is to be noted that radiolabelled derivatives of compound (I), which are suitable for biological studies, are also included within the scope of the present invention.

Suitable salts of the object compound (I) are conventional pharmaceutically acceptable ones and include metal salts such as alkali metal salts (e.g. sodium salt, potassium salt, etc.) and an alkaline earth metal salts (e.g. calcium salt, magnesium salt, etc.), ammonium salts, organic base salts (e.g. trimethylamine salt, triethylamine salt, pyridine salt, picoline salt, dicyclohexylamine salt, N,N′-dibenzylethylenediamine salt, etc.), organic acid salts (e.g. acetate, trifluoroacetate, maleate, tartrate, fumarate, methanesulfonate, benzenesulfonate, formate, toluenesulfonate, etc.), inorganic acid salts (e.g. hydrochloride, hydrobromide, hydroiodide, sulfate, phosphate, etc.), a salt with an amino acid (e.g. arginine, aspartic acid, glutamic acid, etc.), etc. All starting materials and product compounds may be salts. The compounds of above processes can be converted to salts according to a conventional method.

Hereinafter the symbols of the formula (I) are explained in detail. Throughout the specification and claims, the term “lower” is intended to mean 1 to 6 carbon atom(s) unless otherwise indicated.

### (Definition of R¹)

In the formula (I), R¹ is selected from the group consisting of hydrogen, substituted or unsubstituted lower alkyl and substituted or unsubstituted aryl.

Examples of the “lower alkyl” of the “substituted or unsubstituted lower alkyl” for R¹ may include straight or branched (C₁₋₆)alkyl such as methyl, ethyl, propyl, isopropyl, butyl, isobutyl, tert-butyl, pentyl, hexyl, etc., in which the preferred one may be (C₁₋₄)alkyl, and more preferable one may be methyl, ethyl, propyl, isopropyl, isobutyl, etc.

Examples of the substituents for the “substituted lower alkyl” for R¹ may include hydroxy, hydroxy(C₅₋₈)cycloalkyl, (C₅₋₈)cycloalkyl, nitro, nitro (C₅₋₈)cycloalkyl, amido, amido(C₅₋₈)cycloalkyl, sulfonamido, sulfonamido(C₅₋₈)cycloalkyl, ureido, ureido (C₅₋₈)cycloalkyl etc. The number of the substituent may be one; two or more. Where the number of the substituent is two or more, the substituents may be the same or different.

Examples of the “aryl” of the “substituted or unsubstituted aryl” for R¹ may include (C₆₋₁₄)aryl such as phenyl, naphthyl, indenyl, anthryl, etc., in which the preferred one may be (C₆₋₁₀)aryl, and the more preferred one may be phenyl, etc.

Examples of the substituents for the “substituted aryl” for R¹ may include lower alkyl [e.g., (C₁₋₄)alkyl (e.g., methyl, ethyl, propyl, butyl, etc.), etc.], (lower)alkylaminosulfonyl [e.g., (C₁₋₄)alkylaminosulfonyl (e.g., methylaminosulfonyl, ethylaminosulfonyl, propylaminosulfonyl, tert-butylaminosulfonyl, etc.), etc.], aryloxy (e.g., (C₆₋₁₄)aryloxy, etc.), halo(lower)alkyl (e.g., chloromethyl, dichloromethyl, fluoromethyl, difluoromethyl, trifluoromethyl, pentachloroethyl, etc.), hydroxy(lower)alkyl (e.g., hydroxy(C₁₋₄)alkyl, etc.), lower alkanoyl (e.g., (C₁₋₄)alkyl-carbonyl, etc.), halogen (e.g., fluoro, chloro, bromo, iodo, etc.), lower alkoxy (e.g., (C₁₋₄)alkoxy, etc.), carboxy, lower alkoxycarbamoyl, carbamoyl, lower alkylcarbamoyl, etc. The number of the substituent may be one or two or more. Where the number of the substituent is two or more, the substituents may be the same or different.

Suitable examples of R¹ may include hydrogen, methylphenyl, (tert-butylamino)sulfonylphenyl, ethylphenyl, methoxyphenyl, aminosulfonylphenyl, etc.

### (Definition of R²)

In the formula (I), R² is selected from the group consisting of substituted or unsubstituted aryl and substituted or unsubstituted heteroaryl.

Examples of the “aryl” of the “substituted or unsubstituted aryl” for R² may include aryl similar to those exemplified for R¹ above, in which the preferred one may be (C₆₋₁₀)aryl, and the more preferred one may be phenyl, etc.

Examples of the substituents for the “substituted aryl” for R² may include halogen (e.g., fluoro, chloro, bromo, iodo, etc.), lower alkyl [e.g., (C₁₋₄)alkyl (e.g., methyl, ethyl, propyl, butyl, etc.), etc.], lower alkoxy [e.g., (C₁₋₄)alkoxy (e.g., methoxy, ethoxy, propoxy, butoxy, etc.), etc.], halo(lower)alkyl (e.g., chloromethyl, dichloromethyl, fluoromethyl, difluoromethyl, trifluoromethyl, pentachloroethyl, etc.), hydroxy(lower)alkyl, etc. The number of the substituent may be one, two or more. Where the number of the substituent is two or more, the substituents may be the same or different.

Examples of the “heteroaryl” of the “substituted or unsubstituted heteroaryl” for R² may include, 5 to 14-membered heteroaryl, such as furyl, pyrrolyl, thienyl, oxazolyl, etc., in which the preferred one may be 5 or 6-membered heteroaryl, and more preferred one may be thienyl, etc.

Examples of the substituents for the “substituted heteroaryl” for R² may include substituents similar to the substituents exemplified above for the “substituted aryl” for R². The number of the substituent may be one or two or more. Where the number of the substituent is two or more, the substituents may be the same or different.

Suitable examples of R² may include phenyl, fluorophenyl, difluorophenyl, chlorofluorophenyl, methylphenyl, dimethylphenyl, methoxyphenyl, methyl(fluoro)phenyl, etc.

### (Definition of R³)

In the formula (I), R³ is lower alkyl.

Examples of the “lower alkyl” for R³ may include lower alkyl similar to those exemplified for R¹ above, in which the preferred one may be (C₁₋₄)alkyl.

Suitable examples of R³ may include methyl, ethyl, etc.

### (Definition of p)

In the formula (I), p is 0, 1 or 2.

Suitable example of p is 0.

### (Definitions of R⁴ and R⁵)

In the formula (I), R⁴ and R⁵ are each hydrogen or taken together to form a bond.

### (Definitions of R⁶ and R⁷)

In the formula (I), R⁶ and R⁷ are taken together to form a group of the formula:

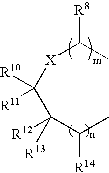

### (Definition of R⁸)

R⁸ is hydrogen.

### (Definition of X)

X is oxygen or N—R⁹, in which R⁹ is hydrogen, substituted or unsubstituted lower alkanoyl, or substituted or unsubstituted lower alkyl.

Examples of the “lower alkyl” of the “substituted or unsubstituted lower alkyl” for R⁹ may include lower alkyl similar to those exemplified for R¹ above.

Examples of the substituents for the “substituted lower alkyl for R⁹ may include those exemplified as the substituents for the “substituted lower alkyl” for R¹⁸ and R¹⁹ mentioned below, in which the preferred are carboxy, hydroxy, (C₁₋₆)alkoxycarbonyl, morpholino, morpholinocarbonyl or (C₁₋₆)alkylsulfonyloxy.

Examples of the “lower alkanoyl” of the “substituted or unsubstituted lower alkanoyl” for R⁹ may include (C₂₋₇)alkanoyl [e.g, (C₁₋₆)alkyl-carbonyl (e.g. acetyl, ethylcarbonyl, propylcarbonyl, butylcarbonyl, pentylcarbonyl, hexylcarbonyl, etc.), etc.].

Examples of the substituents for the “substituted lower alkanoyl” for R⁹ may include those exemplified as the substituents for the “substituted lower alkyl” for R¹⁸ and R¹⁹ mentioned below.

Preferred examples of R⁹ may include hydrogen; (C₁₋₆)alkyl optionally substituted by carboxy, hydroxy, (C₁₋₆)alkoxycarbonyl, morpholino, morpholinocarbonyl or (C₁₋₆)alkylsulfonyloxy; (C₂₋₇)alkanoyl, etc.

Alternatively, R⁶ and R⁹ may be taken together to form a bond.

### (Definitions of m and n)

m and n are each 0, 1 or 2.

### (Definitions of R¹⁰ and R¹¹)

In the formula (I), R¹⁰ is selected from the group consisting of hydrogen, halogen, hydroxy, formyl, cyano, substituted or unsubstituted lower alkyl, substituted or unsubstituted amino, substituted or unsubstituted lower alkoxy, saturated cyclic amino, substituted or unsubstituted carbamoyl, carboxy and substituted or unsubstituted lower alkoxycarbony.

Specifically, R¹⁰ is hydrogen or substituted or unsubstituted lower alkyl.

Examples of the “lower alkyl” for the “substituted or unsubstituted lower alkyl” for R¹⁰ may include lower alkyl similar to those exemplified for R¹ above, in which the preferred one may be (C₁₋₆)alkyl and more preferred one may be methyl, ethyl, isopropyl, etc.

Examples of the substituents for the “substituted lower alkyl” for R¹⁰ may include:

(1) hydroxy;
(2) arylalkoxy [e.g., (C₆₋₁₄)aryl(C₁₋₆)alkoxy such as benzyloxy, phenethyloxy, etc.];
(3) di(C₆₋₁₄)aryl(C₁₋₆)alkylsilyloxy (e.g., methyldiphenylsilyloxy, tert-butyldiphenylsilyloxy, etc.), etc.

Preferred examples of R¹⁰ may include hydrogen, (C₁₋₆)alkyl optionally substituted by (C₆₋₁₄)aryl(C₁₋₆)alkoxy, di(C₆₋₁₄)aryl(C₁₋₆)alkylsilyloxy or hydroxy, etc.

Examples of the “substituted or unsubstituted amino”, “substituted or unsubstituted lower alkoxy”, “saturated cyclic amino”, “substituted or unsubstituted carbamoyl” and “lower alkoxycarbonyl” for R¹⁰ may be similar to the “substituted or unsubstituted amino”, “substituted or unsubstituted lower alkoxy”, “saturated cyclic amino”, “substituted or unsubstituted carbamoyl” and “lower alkoxycarbonyl” exemplified above as the substituents for the “substituted lower alkyl” for R¹² mentioned below.

Alternatively, R⁹ and R¹⁰ may be taken together to form lower alkylene (e.g., (C₂₋₆)alkylene such as ethylene, propylene, butylene, pentylene, hexylene, etc.), in which preferred may be propylene, etc.

R¹¹ is selected from the group consisting of hydrogen, halogen, substituted or unsubstituted lower alkyl, carboxy and substituted or unsubstituted lower alkoxycarbonyl.

Examples of the “halogen” for R¹¹ may include chloro, fluoro, bromo, iodo, etc.

Examples of the “lower alkyl” for the “substituted or unsubstituted lower alkyl” for R¹¹ may include lower alkyl similar to those exemplified for R¹ above, and examples of the “lower alkoxycarbonyl” for the “substituted or unsubstituted lower alkoxycarbonyl” for R¹¹ may include those exemplified above as the substituent (8) for the “substituted lower alkyl” for R¹² mentioned below. Examples of the substituents for “substituted lower alkyl” and “substituted lower alkoxycarbonyl” for R¹¹ may include those exemplified as the substituents for the “substituted lower alkyl” for R¹.

Specifically, R¹¹ is hydrogen, or lower alkyl.

Examples of the lower alkyl for R¹¹ may include lower alkyl similar to those exemplified for R¹ above, in which the preferred may be (C₁₋₄)alkyl and more preferred may be methyl, ethyl, isopropyl, etc.

Alternatively, R¹⁰ and R¹¹ may be taken together to form

(1) substituted or unsubstituted lower alkylene [e.g., (C₂₋₆)alkylene (e.g., ethylene, propylene, butylene, pentylene, hexylene, etc., in which the preferred one may be ethylene, propylene, butylene, etc.)];
(2) substituted or unsubstituted lower alkylidene [e.g., (C₁₋₆)alkylidene such as methylidene, ethylidene, propylidene, butylidene, pentylidene, hexylene, etc., in which the preferred one may be methylidene, ethylidene, propan-2-ylidene, etc.];
(3) oxo, or
(4) hydroxyimino, etc.

As used herein, the term “lower alkylene” in the phrase “substituted lower alkylene” formed by R¹⁰ and R¹¹ may also include alkylene group as defined above in which one or more carbon atom(s) is (are) replaced by one or more heteroatom(s) selected from a nitrogen atom, an oxygen atom and a sulfur atom, and examples of such lower alkylene formed by R¹⁰ and R¹¹ may include following groups such as, but not limited to, —(CH₂)₂—O—(CH₂)₂—, —(CH₂)₂—N—(CH₂)₂—, etc.

Examples of the substituents for the above-mentioned “substituted lower alkylene” formed together by R¹⁰ and R¹¹ may include:

(1) arylalkoxycarbonyl [e.g., (C₆₋₁₄)aryl(C₁₋₆)alkoxycarbonyl such as benzyloxycarbonyl, phenetyloxycarbonyl, etc.];
(2) acyl [e.g., (C₁₋₇)alkanoyl such as formyl, acetyl, propionyl, butyryl, etc., (C₆₋₁₄)acyl such as benzoyl, etc.], etc.

Preferred examples of the “substituted or unsubstituted lower alkylene” formed by R¹⁰ and R¹¹ may include (C₂₋₆)alkylene in which one or more carbon atom(s) may be replaced with heteroatom(s) selected from an oxygen atom and a nitrogen atom, which is optionally substituted by (C₆₋₁₄)aryl(C₁₋₆)alkoxycarbonyl or (C₁₋₇)alkanoyl.

Alternatively, R⁹ and R¹⁰ may be taken together to form lower alkylene or a bond.

Examples of the “lower alkylene” formed by R⁹ and Rⁿ may include (C₂₋₆)alkylene, in which preferred are propylene, etc.

### (Definitions of R¹², R¹³ and R¹⁴)

In the above-mentioned formula (I), R¹² is selected from the group consisting of hydrogen, halogen, hydroxy, formyl, cyano, substituted or unsubstituted lower alkyl, substituted or unsubstituted amino, substituted or unsubstituted lower alkoxy, saturated cyclic amino, substituted or unsubstituted carbamoyl, carboxy and substituted or unsubstituted lower alkoxycarbonyl, substituted or unsubstituted acyloxy.

Examples of the “halogen” for R¹² may include chloro, fluoro, bromo, iodo, etc., in which the preferred one may be fluoro, etc.

Examples of the “lower alkyl” of the “substituted or unsubstituted lower alkyl” for R¹² may include lower alkyl similar to those exemplified above for R¹, in which the preferred one may be (C₁₋₄)alkyl and more preferred one may be methyl, ethyl, isopropyl, etc.

Examples of the substituents for the “substituted lower alkyl” for R¹² may include:

(1) hydroxy, hydroxyimino or tri(lower)alkylsilyloxy;
(2) halogen (e.g., chloro, fluoro, bromo, iodo, etc.);
(3) substituted or unsubstituted amino [e.g., amino, mono- or di-(substituted or unsubstituted lower alkyl)amino (e.g., mono-(C₁₋₆)alkylamino in which said (C₁₋₆)alkyl may be substituted by (C₆₋₁₄)aryl, (C₃₋₈)cycloalkylcarbonyl or hydroxy (e.g., methylamino, ethylamino, propylamino, isopropylamino, butylamino, tert-butylamino, neopentylamino, hydroxymethylamino, hydroxyethylamino, cyclopropanecarbonylamino, etc.), di-(C₁₋₄)alkylamino in which one or both of said (C₁₋₄)alkyl may be substituted by (C₆₋₁₄)aryl (e.g., dimethylamino, diethylamino, ethylmethylamino, etc.), 2-hydroxyethylamino, 2-methoxyethylamino, 2-(dimethylamino)ethylamino, 2-hydroxy-1,1-dimethylethylamino, 2-hydroxy-1-(hydroxymethyl)ethylamino, (2-hydroxyethyl)methylamino, (2-methoxyethyl)methylamino, benzylmethylamino, tert-butylbenzylamino, dibenzylamino etc.), mono-(C₂₋₇) alkanoylamino (e.g., acetylamino, ethylcarbonylamino, propylcarbonylamino, isopropylcarbonylamino, butylcarbonylamino, pentylcarbonylamino, hexylcarbonylamino, etc.), (C₃₋₈)cycloalkylamino (e.g., cyclopropylamino, cyclobutylamino, cyclopentylamino, cyclohexylamino, etc.), etc.];
(4) substituted or unsubstituted lower alkoxy (e.g., (C₁₋₆)alkoxy (e.g., methoxy, ethoxy, propoxy, isopropoxy, butoxy, neopentyloxy, etc.), (C₆₋₁₄)aryl(C₁₋₆)alkoxy (e.g., benzyloxy, etc.), 2-hydroxyethyloxy, 2-hydroxy-1,1-dimethylethyloxy, 2-methoxyethyloxy, 2-(dimethylamino)ethyloxy, etc.);
(5) saturated cyclic amino [e.g., 4-, 5- or 6-membered saturated cyclic amino which may further have heteroatom(s) selected from a nitrogen atom, an oxygen atom and a sulfur atom and/or oxo besides the amino nitrogen and may have substituent(s), such as azetidinyl (e.g., 3-hydroxy-1-azetidinyl, 3-amino-1-azetidinyl, 3-methylamino-1-azetidinyl, etc.), pyrrolidinyl (e.g., 1-pyrrolidinyl, 3-hydroxy-1-pyrrolidinyl, 3-amino-1-pyrrolidinyl, 3-methylamino-1-pyrrolidinyl, etc.), morpholinyl (e.g., morpholino, etc.), 4-(lower)alkyl-1-piperazinyl (e.g., 4-methyl-1-piperazinyl, 4-isopropyl-1-piperazinyl, etc.), 4-(mono- or di-(lower)alkylamino)-1-piperidinyl (e.g., 4-(dimethylamino)-1-piperidinyl, etc.), oxopyrrolidinyl (e.g., 2-oxo-1-pyrrolidinyl, etc.), etc.];
(6) substituted or unsubstituted carbamoyl [e.g., carbamoyl, (lower)alkylcarbamoyl (e.g., (C₁₋₄)alkylcarbamoyl such as methylcarbamoyl, ethylcarbamoyl, propylcarbamoyl, isopropylcarbamoyl, butylcarbamoyl, etc.), (C₃₋₈)cycloalkylcarbamoyl (e.g., cyclopropylcarbamoyl, etc.), etc.];
(7) carboxy;
(8) lower alkoxycarbonyl [e.g., (C₁₋₆)alkoxycarbonyl (e.g., methoxycarbonyl, ethoxycarbonyl, propyloxycarbonyl, tert-butoxycarbonyl, pentyloxycarbamoyl, hexyloxycarbamoyl, etc.), etc.];
(9) lower alkylureido [e.g., (C₁₋₆)alkylureido (e.g., methylureido, ethylureido, etc.)]
(10) lower acyloxy [e.g., (C₁₋₇)alkanoyloxy (e.g., formyloxy, acetyloxy, ethylcarbonyloxy, propylcarbonyloxy, butylcarbonyloxy, pentylcarbonyloxy, hexylcarbonyloxy, etc.], etc.

The number of the substituent may be one, two or more. Where the number of the substituent is two or more, the substituents may be the same or different.

Examples of the “substituted or unsubstituted amino”, “saturated cyclic amino”, “substituted or unsubstituted lower alkoxy”, “substituted or unsubstituted carbamoyl” and “lower alkoxycarbonyl” for R¹² may be similar to the “substituted or unsubstituted amino”, “saturated cyclic amino”, “substituted or unsubstituted lower alkoxy”, “substituted or unsubstituted carbamoyl” and “substituted or unsubstituted lower alkoxycarbonyl” exemplified above as the substituents of the “substituted lower alkyl” for R¹².

Examples of the “acyloxy” for the “substituted or unsubstituted acyloxy” for R¹² may include lower acyloxy similar to those exemplified above as the substituent (10) for the “substituted lower alkyl” for R¹² mentioned above.

Examples of the substituents for the “substituted acyloxy” for R¹² may be similar to those exemplified as the substituents for the “substituted lower alkyl” for R¹².

Preferable examples for R¹² may include hydrogen; halogen; hydroxy; carboxy; formyl; cyano; hydroxycyano; (C₁₋₆)alkyl optionally substituted by hydroxy, hydroxyimino, halogen, (C₁₋₆)alkoxy, (C₁₋₇)alkanoyloxy, amino, mono- or di-(C₁₋₆)alkylamino (in which one or both of said (C₁₋₆)alkyl is (are) optionally substituted by hydroxy, (C₁₋₆)alkoxy, (C₆₋₁₄)aryl or (C₃₋₆)cycloalkyl-carbonyl), (C₁₋₆)alkylureido, morpholino, (C₁₋₇)alkanoyloxy, or 4- to 6-membered cyclic amino optionally substituted by hydroxy, (C₁₋₆)alkyl or di(C₁₋₆)alkylamino; mono- or di-(C₁₋₇)alkylamino; 4- to 6-membered cyclic amino; (C₁₋₆)alkoxy optionally substituted by (C₆₋₁₄)aryl; carbamoyl optionally substituted by (C₃₋₆)cycloalkyl or hydroxy(C₁₋₆)alkyl; (C₁₋₆)alkoxycarbonyl; (C₁₋₆)alkoxycarbonyloxy, etc.

Among the above-mentioned substituents, suitable examples of R¹² may include hydrogen, fluoro, hydroxy, formyl, cyano, methyl, aminomethyl, tert-butylaminomethyl, dimethylaminomethyl, diethylaminomethyl, dibenzylaminomethyl, benzylmethylaminomethyl, benzyl(tert-buthyl)aminomethyl, methoxycarbonylmethyl, 3-hydroxyazetinylmethyl, 4-methylpiperazinylmethyl, pyrrolidinylmethyl, hydroxymethyl, hydroxyethylaminomethyl, methoxyethylaminomethyl, iodomethyl, methylaminomethyl, morpholinomethyl, (2-hydroxyethyl)methylaminomethyl, acetyloxymethyl, 4-(dimethylamino)-1-piperidinylmethyl, ethoxycarbonylmethyl, cyclopropylcarbamoylmethyl, ethylureidomethyl, hydroxyiminomethyl, dimethylamino, isopropylamino, 3-hydroxy-1-azetidinyl, piperidino, morpholino, benzyloxy, neopentyloxy, carboxy, methoxycarbonyl, ethoxycarbonyl, tert-butoxycarbonyl, carbamoyl, cyclopropylcarbamoyl, etc.

R¹³ is selected from the group consisting of hydrogen, halogen, substituted or unsubstituted lower alkyl, carboxy and substituted or unsubstituted lower alkoxycarbonyl.

Examples of the “halogen” and “substituted or unsubstituted lower alkoxycarbonyl” for R¹³ may be similar to those exemplified for R¹¹.

Examples of the “lower alkyl” of the “substituted or unsubstituted lower alkyl” for R¹³ may include lower alkyl similar to those exemplified above for R¹, in which the preferred one may be (C₁₋₄)alkyl, and more preferred one may be methyl, ethyl, isopropyl, etc.

Examples of the substituents for the “substituted lower alkyl” for R¹³ may include

(1) hydroxy;
(2) halogen (e.g., chloro, fluoro, bromo, iodo, etc.);
(3) substituted or unsubstituted amino [e.g., amino, mono- or di-(substituted or unsubstituted lower alkyl)amino (e.g., mono-(C₁₋₆)alkylamino (e.g., methylamino, ethylamino, propylamino, isopropylamino, butylamino, tert-butylamino, neopentylamino, etc.), di-(C₁₋₄)alkylamino (e.g., dimethylamino, diethylamino, ethylmethylamino, etc.), 2-hydroxyethylamino, 2-methoxyethylamino, 2-(dimethylamino)ethylamino, 2-hydroxy-1,1-dimethylethylamino, 2-hydroxy-1-(hydroxymethyl)ethylamino, (2-hydroxyethyl)methylamino, (2-methoxyethyl)methylamino, etc.), mono-(C₂₋₇)alkanoylamino (e.g., acetylamino, ethylcarbonylamino, propylcarbonylamino, isopropylcarbonylamino, butylcarbonylamino, pentylcarbonylamino, hexylcarbonylamino, etc.), (C₃₋₈)cycloalkylamino (e.g., cyclopropylamino, cyclobutylamino, cyclopentylamino, cyclohexylamino, etc.), etc.];
(4) substituted or unsubstituted lower alkoxy [e.g., (C₁₋₄)alkoxy (e.g., methoxy, ethoxy, propoxy, isopropoxy, butoxy, etc.), 2-hydroxyethyloxy, 2-hydroxy-1,1-dimethylethyloxy, 2-methoxyethyloxy, 2-(dimethylamino)ethyloxy, etc.];
(5) lower alkanoyloxy [e.g., (C₁₋₇)alkanoyloxy [e.g., formyloxy, acetyloxy, ethylcarbonyloxy, propylcarbonyloxy, butylcarbonyloxy, pentylcarbonyloxy, hexylcarbonyloxy, etc.]; etc.

The number of the substituent may be one, two or more. Where the number of the substituent is two or more, the substituents may be the same or different.

Suitable examples of R¹³ may include hydrogen, halogen (e.g., fluoro, etc.), (C₁₋₆)alkyl optionally substituted by hydroxy, fluoro, halogen, (C₁₋₆)alkoxy or (C₁₋₇)alkanoyl (e.g., methyl, hydroxymethyl, fluoromethyl, methoxymethyl, acetyloxymethyl, etc.), in which preferred are hydrogen, halogen or (C₁₋₆)alkyl optionally substituted by hydroxy or (C₁₋₇)alkanoyloxy (e.g., hydroxymethyl, acetyloxymethyl, etc.), etc.

R¹⁴ is selected from the group consisting of hydrogen, halogen, substituted or unsubstituted lower alkyl, carboxy and substituted or unsubstituted lower alkoxycarbonyl.

The “halogen”, “substituted or unsubstituted lower alkyl” and “substituted or unsubstituted lower alkoxycarbonyl” for R¹⁴ may be similar to those exemplified for R¹¹.

Preferably, R¹⁴ is hydrogen.

Alternatively, R¹² and R¹³ may be taken together to form (1) substituted or unsubstituted lower alkylene [e.g., (C₂₋₆)alkylene (e.g., ethylene, propylene, butylene, pentylene, hexylene, etc., in which the preferred one may be ethylene, propylene, butylene, etc.)];

(2) substituted or unsubstituted lower alkylidene (e.g., (C₁₋₆)alkylidene such as methylidene, ethylidene, propylidene, butylidene, pentylidene, hexylidene, etc., in which the preferred one may be methylidene, ethylidene, propan-2-ylidene, etc.];
(3) oxo, or
(4) hydroxyimino.

The term “lower alkylene” in the phrase “substituted or unsubstituted lower alkylene” for R¹² and R¹³ refers to alkylene group as defined above in which one or more carbon atom(s) is (are) replaced by one or more heteroatom(s) selected from a nitrogen atom, an oxygen atom and a sulfur atom

Examples of the substituents for the above-mentioned “substituted lower alkylene” formed by R¹² and R¹³ may include

(1) substituents for “substituted or unsubstituted lower alkyl” for R¹²; and
(2) substituted or unsubstituted lower alkyl [e.g., substituted or unsubstituted (C₁₋₆)alkyl (e.g., methyl, ethyl, propyl, isopropyl, n-butyl, tert-butyl, pentyl, hexyl, etc.), examples of the substituent may include the substituents for the “substituted or unsubstituted lower alkyl” for R¹²]

Suitable examples of the “substituted or unsubstituted lower alkylene” formed by R¹² and R¹³ may include following groups such as, but not limited to,

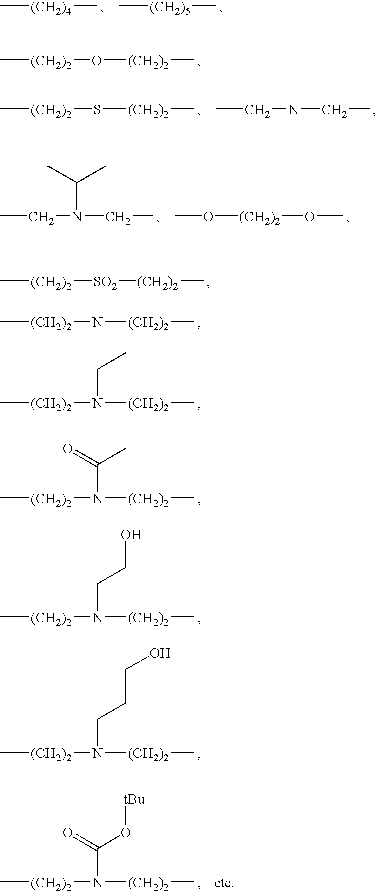

Examples of the substituents for the above-mentioned “substituted lower alkylidene” formed by R¹² and R¹³ may be similar to those exemplified for the “substituted or unsubstituted alkylene” formed by R¹² and R¹³.

Suitable examples of the “substituted or unsubstituted lower alkylidene” formed by R¹² and R¹³ may include (C₁₋₆)alkylidene optionally substituted by hydroxy, such as the following groups, but not limited to, —CH₂═CH—CH₃═CH—CH₂—OH, etc.

Alternatively, R¹¹ and R¹² or R¹³ and R¹⁴ may be taken together to form a bond.

In an embodiment of the present invention, R⁶ and R⁷ are taken together to form the following structure (A), (B1) or (B2).

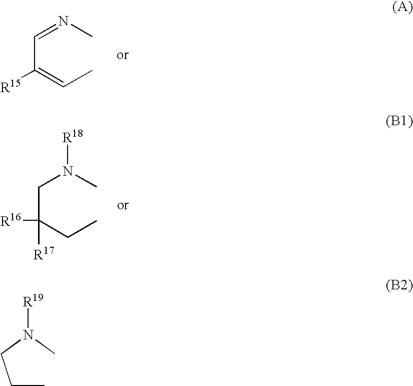

### (Definition of R¹⁵)

In the above-mentioned formula (A), R¹⁵ is selected from the group consisting of hydroxy, substituted or unsubstituted lower alkyl, substituted or unsubstituted amino, substituted or unsubstituted lower alkoxy, saturated cyclic amino, lower substituted or unsubstituted carbamoyl, carboxy and substituted or unsubstituted lower alkoxycarbonyl.

Examples of the “lower alkyl” of the “substituted or unsubstituted lower alkyl” for R¹⁵ may include lower alkyl similar to those exemplified for R¹ above, in which the preferred one may be (C₁₋₄)alkyl and more preferred one may be methyl, ethyl, isopropyl, etc.

Examples of the substituents for the “substituted lower alkyl” for R¹⁵ may include:

(1) hydroxy;
(2) substituted or unsubstituted amino [e.g., amino, mono or di-(substituted or unsubstituted lower alkyl)amino (e.g., mono-(C₁₋₆)alkylamino such as methylamino, ethylamino, propylamino, isopropylamino, butylamino, tert-butylamino, neopentylamino, etc.; di-(C₁₋₄)alkylamino such as dimethylamino, diethylamino, ethylmethylamino, etc.; 2-hydroxyethylamino, 2-methoxyethylamino, 2-(dimethylamino)ethylamino, 2-hydroxy-1,1-dimethylethylamino, 2-hydroxy-1-(hydroxymethyl)ethylamino, (2-hydroxyethyl)methylamino, (2-methoxyethyl)methylamino, etc.), mono-(C₂₋₅)alkanoylamino (e.g., acetylamino, ethylcarbonylamino, propylcarbonylamino, isopropylcarbonylamino, butylcarbonylamino, etc.), (C₃₋₆)cycloalkylamino (e.g., cyclopropylamino, cyclobutylamino, cyclopentylamino, cyclohexylamino, etc.), etc.);
(3) substituted or unsubstituted lower alkoxy [e.g., (C₁₋₄)alkoxy (e.g., methoxy, ethoxy, propoxy, isopropoxy, butoxy, etc.), 2-hydroxyethyloxy, 2-hydroxy-1,1-dimethylethyloxy, 2-methoxyethyloxy, 2-(dimethylamino)ethyloxy, etc.];
(4) saturated cyclic amino [e.g., 4-, 5- or 6-membered saturated cyclic amino which may further have heteroatom(s) selected from a nitrogen atom, an oxygen atom and a sulfur atom and/or oxo besides the amino nitrogen and may have substituent(s), such as azetidinyl (e.g., 3-hydroxy-1-azetidinyl, 3-amino-1-azetidinyl), pyrrolidinyl (e.g., 1-pyrrolidinyl, etc.), morpholinyl (e.g., morpholino, etc.), 4-(lower)alkyl-1-piperazinyl (e.g., 4-methyl-1-piperazinyl, 4-isopropyl-1-piperazinyl, etc.), oxopyrrolidinyl (e.g., 2-oxo-1-pyrrolidinyl, etc.), etc.];
(5) substituted or unsubstituted carbamoyl [e.g., carbamoyl, (lower)alkylcarbamoyl (e.g., (C₁₋₄)alkylcarbamoyl such as methylcarbamoyl, ethylcarbamoyl, propylcarbamoyl, isopropylcarbamoyl, butylcarbamoyl, etc.), etc.],
(6) carboxy;
(7) lower alkoxycarbonyl [e.g., (C₁₋₆)alkoxycarbonyl (e.g., methoxycarbonyl, ethoxycarbonyl, tert-butoxycarbonyl, pentyloxycarbonyl, hexyloxycarbonyl), etc.], etc. The number of the substituent may be one, two or more. Where the number of the substituent is two or more, the substituents may be the same or different.

Examples of the “substituted or unsubstituted amino”, “substituted or unsubstituted lower alkoxy”, “saturated cyclic amino”, “substituted or unsubstituted carbamoyl” and “lower alkoxycarbonyl” for R¹⁵ may be similar to the “substituted or unsubstituted amino”, “substituted or unsubstituted lower alkoxy”, “saturated cyclic amino”, “substituted or unsubstituted carbamoyl” and “lower alkoxycarbonyl” exemplified above as the substituents for the “substituted lower alkyl” for R¹⁵.

Suitable examples of R¹⁵ may include dimethylaminomethyl, methylaminomethyl, hydroxymethyl, morpholino, 3-hydroxyl-azetidinyl, etc.

### (Definitions of R¹⁶ and R¹⁷)

In the above-mentioned formula (B1), R¹⁶ is selected from the group consisting of hydrogen, halogen, hydroxy, substituted or unsubstituted lower alkyl, substituted or unsubstituted amino, saturated cyclic amino, substituted or unsubstituted lower alkoxy, substituted or unsubstituted carbamoyl, carboxy and lower alkoxycarbonyl.

Examples of the “halogen” for R¹⁶ may include chloro, fluoro, bromo, iodo, etc., in which the preferred one may be fluoro, etc.

Examples of the “lower alkyl” of the “substituted or unsubstituted lower alkyl” for R¹⁶ may include lower alkyl similar to those exemplified for R¹ above, in which the preferred one may be (C₁₋₄)alkyl and more preferred one may be methyl, ethyl, isopropyl, etc.

Examples of the substituents for the “substituted lower alkyl” for R¹⁶ may include:

(1) hydroxy or tri(lower)alkylsilyloxy;
(2) halogen (e.g., chloro, fluoro, bromo, iodo, etc.);
(3) substituted or unsubstituted amino [e.g., amino, mono- or di-(substituted or unsubstituted lower alkyl)amino (e.g., mono-(C₁₋₆)alkylamino (e.g., methylamino, ethylamino, propylamino, isopropylamino, butylamino, tert-butylamino, neopentylamino, etc.), di-(C₁₋₄)alkylamino (e.g., dimethylamino, diethylamino, ethylmethylamino, etc.), 2-hydroxyethylamino, 2-methoxyethylamino, 2-(dimethylamino)ethylamino, 2-hydroxy-1,1-dimethylethylamino, 2-hydroxy-1-(hydroxymethyl)ethylamino, (2-hydroxyethyl)methylamino, (2-methoxyethyl)methylamino, etc.), mono-(C₂₋₅)alkanoylamino (e.g., acetylamino, ethylcarbonylamino, propylcarbonylamino, isopropylcarbonylamino, butylcarbonylamino, etc.), (C₃₋₈) cycloalkylamino (e.g., cyclopropylamino, cyclobutylamino, cyclopentylamino, cyclohexylamino, etc.), etc.];
(4) substituted or unsubstituted lower alkoxy (e.g., (C₁₋₄)alkoxy (e.g., methoxy, ethoxy, propoxy, isopropoxy, butoxy, etc.), 2-hydroxyethyloxy, 2-hydroxy-1,1-dimethylethyloxy, 2-methoxyethyloxy, 2-(dimethylamino)ethyloxy, etc.);
(5) saturated cyclic amino [e.g., 4-, 5- or 6-membered saturated cyclic amino which may further have heteroatom(s) selected from a nitrogen atom, an oxygen atom and a sulfur atom and/or oxo besides the amino nitrogen and may have substituent(s), such as azetidinyl (e.g., 3-hydroxy-1-azetidinyl, 3-amino-1-azetidinyl, 3-methylamino-1-azetidinyl, etc.), pyrrolidinyl (e.g., 1-pyrrolidinyl, 3-hydroxy-1-pyrrolidinyl, 3-amino-1-pyrrolidinyl, 3-methylamino-1-pyrrolidinyl, etc.), morpholinyl (e.g., morpholino, etc.), 4-(lower)alkyl-1-piperazinyl (e.g., 4-methyl-1-piperazinyl, 4-isopropyl-1-piperazinyl, etc.), 4-(mono- or di-(lower)alkylamino)-1-piperidinyl (e.g., 4-(dimethylamino)-1-piperidinyl, etc.), oxopyrrolidinyl (e.g., 2-oxo-1-pyrrolidinyl, etc.), etc.];
(6) substituted or unsubstituted carbamoyl [e.g., carbamoyl, (lower)alkylcarbamoyl (e.g., (C₁₋₄)alkylcarbamoyl such as methylcarbamoyl, ethylcarbamoyl, propylcarbamoyl, isopropylcarbamoyl, butylcarbamoyl, etc.), etc.];
(7) carboxy;
(8) lower alkoxycarbonyl [e.g., (C₁₋₄)alkoxycarbonyl (e.g., methoxycarbonyl, ethoxycarbonyl, tert-butoxycarbonyl, etc.), etc.], etc. The number of the substituent may be one or two or more. Where the number of the substituent is two or more, the substituents may be the same or different.

Examples of the “substituted or unsubstituted amino”, “saturated cyclic amino”, “substituted or unsubstituted lower alkoxy”, “substituted or unsubstituted carbamoyl” and “lower alkoxycarbonyl” for R¹⁶ may be similar to the “substituted or unsubstituted amino”, “saturated cyclic amino”, “substituted or unsubstituted lower alkoxy”, “substituted or unsubstituted carbamoyl” and “lower alkoxycarbonyl” exemplified as the substituents of the “substituted or unsubstituted lower alkyl” for R⁷.

Suitable examples of R¹⁶ may include hydrogen, fluoro, hydroxy, dimethylaminomethyl, hydroxymethyl, iodomethyl, 4-(dimethylamino)-1-piperidinylmethyl, dimethylamino, piperidino, isopropylamino, methylaminomethyl, morpholinomethyl, (2-hydroxyethyl)methylaminomethyl, morpholino, carboxy, methoxycarbonyl, tert-butoxycarbonyl, 3-hydroxy-1-azetidinyl, etc.

In the above-mentioned formula (B1), R¹⁷ is selected from the group consisting of hydrogen, halogen, substituted or unsubstituted lower alkyl, carboxy and lower alkoxycarbonyl.

Examples of the “halogen” for R¹⁷ may include chloro, fluoro, bromo, iodo, etc., in which the preferred one may be fluoro, etc.

Examples of the “lower alkyl” of the “substituted or unsubstituted lower alkyl” for R¹⁷ may include lower alkyl similar to those exemplified for R¹ above, in which the preferred one may be (C₁₋₄)alkyl, and more preferred one may be methyl, ethyl, isopropyl, etc.

Examples of the substituents for the “lower alkyl” for R¹⁷ may include

(1) hydroxy;
(2) halogen (e.g., chloro, fluoro, bromo, iodo, etc.);
(3) substituted or unsubstituted amino [e.g., amino, mono- or di-(substituted or unsubstituted lower alkyl)amino (e.g., mono-(C₁₋₆)alkylamino (e.g., methylamino, ethylamino, propylamino, isopropylamino, butylamino, t-butylamino, neopentylamino, etc.), di-(C₁₋₄)alkylamino (e.g., dimethylamino, diethylamino, ethylmethylamino, etc.), 2-hydroxyethylamino, 2-methoxyethylamino, 2-(dimethylamino)ethylamino, 2-hydroxy-1,1-dimethylethylamino, 2-hydroxy-1-(hydroxymethyl)ethylamino, (2-hydroxyethyl)methylamino, (2-methoxyethyl)methylamino, etc.), mono-(C₂₋₅)alkanoylamino (e.g., acetylamino, ethylcarbonylamino, propylcarbonylamino, isopropylcarbonylamino, butylcarbonylamino, etc.), (C₃₋₈) cycloalkylamino (e.g., cyclopropylamino, cyclobutylamino, cyclopentylamino, cyclohexylamino, etc.), etc.];
(4) substituted or unsubstituted lower alkoxy [e.g., (C₁₋₄)alkoxy (e.g., methoxy, ethoxy, propoxy, isopropoxy, butoxy, etc.), 2-hydroxyethyloxy, 2-hydroxy-1,1-dimethylethyloxy, 2-methoxyethyloxy, 2-(dimethylamino)ethyloxy, etc.], etc. The number of the substituent may be one or two or more. Where the number of the substituent is two or more, the substituents may be the same or different.

Suitable examples of R¹⁷ may include hydrogen, methyl, hydroxymethyl, fluoro, fluoromethyl, methoxymethyl, etc.

Alternatively, R¹⁶ and R¹⁷ are taken together to form lower alkylene or lower alkylidene.

Examples of the “lower alkylene” for R¹⁶ and R¹⁷ may include (C₂₋₆)alkylene such as ethylene, propylene, butylene, pentylene, hexylene, etc., in which the preferred one may be ethylene, propylene, butylene, etc.

Examples of the “lower alkylidene” for R¹⁶ and R¹⁷ may include (C₁₋₆)alkylidene such as methylidene, ethylidene, propylidene, butylidene, pentylidene, hexylene, etc., in which the preferred one may be methylidene, ethylidene, propan-2-ylidene, etc.

### (Definition of R¹⁸)

In the above-mentioned formula (B1), R¹⁸ is hydrogen or substituted or unsubstituted lower alkyl; provided that when both R¹⁶ and R¹⁷ are simultaneously hydrogen, R¹ is substituted or unsubstituted lower alkyl.

Examples of the “lower alkyl” of the “substituted or unsubstituted lower alkyl” for R¹⁸ may include lower alkyl similar to those exemplified for R¹ above, in which the preferred one may be (C₁₋₄)alkyl and more preferred one may be ethyl, propyl, etc.

Examples of the substituents for the “substituted lower alkyl” for R¹⁸ may include

(1) hydroxy;
(2) carboxy;
(3) halogen (chloro, fluoro, bromo, iodo);
(4) (lower)alkoxycarbonyl [e.g., (C₁₋₆)alkoxycarbonyl (e.g., methoxycarbonyl, ethoxycarbonyl, propoxycarbonyl, butoxycarbonyl, t-butoxycarbonyl, pentyloxycarbonyl, hexyloxycarbonyl, etc.), etc.];
(5) substituted or unsubstituted amino (e.g., amino, mono- or di-(substituted or unsubstituted lower alkyl)amino (e.g., mono-(C₁₋₆)alkylamino (e.g., methylamino, ethylamino, propylamino, isopropylamino, butylamino, tert-butylamino, neopentylamino, etc.), di-(C₁₋₄)alkylamino (e.g., dimethylamino, diethylamino, ethylmethylamino, etc.), 2-hydroxyethylamino, 2-methoxyethylamino, 2-(dimethylamino)ethylamino, 2-hydroxy-1,1-dimethylethylamino, 2-hydroxy-1-(hydroxymethyl)ethylamino, (2-hydroxyethyl)methylamino, (2-methoxyethyl)methylamino, etc.), mono-(C₂₋₅)alkanoylamino (e.g., acetylamino, ethylcarbonylamino, propylcarbonylamino, isopropylcarbonylamino, butylcarbonylamino, etc.), (C₃₋₉)cycloalkylamino (e.g., cyclopropylamino, cyclobutylamino, cyclopentylamino, cyclohexylamino, etc.), etc.];
(6) substituted or unsubstituted lower alkoxy [e.g., (C₁₋₄)alkoxy (e.g., methoxy, ethoxy, propoxy, isopropoxy, butoxy, etc.), 2-hydroxyethyloxy, 2-hydroxy-1,1-dimethylethyloxy, 2-methoxyethyloxy, 2-(dimethylamino)ethyloxy, etc.];
(7) saturated cyclic amino [e.g., 4, 5- or 6-membered saturated cyclic amino which may further have heteroatom(s) selected from a nitrogen atom, an oxygen atom and a sulfur atom and/or oxo besides the amino nitrogen and may have substituent(s), such as azetidinyl (e.g., 3-hydroxy-1-azetidinyl, 3-amino-1-azetidinyl, 3-methylamino-1-azetidinyl, etc.), pyrrolidinyl (e.g., 1-pyrrolidinyl, 3-hydroxy-1-pyrrolidinyl, 3-amino-1-pyrrolidinyl, 3-methylamino-1-pyrrolidinyl, etc.), morpholinyl (e.g., morpholino, etc.), 4-(lower)alkyl-1-piperazinyl (e.g., 4-methyl-1-piperazinyl, 4-isopropyl-1-piperazinyl, etc.), 4-(mono- or di-(lower)alkylamino)-1-piperidinyl (e.g., 4-(dimethylamino)-1-piperidinyl, etc.), oxopyrrolidinyl (e.g., 2-oxo-1-pyrrolidinyl, etc.), etc.];
(8) lower alkylsulfonyloxy [e.g., (C₁₋₆)alkylsulfonyloxy (e.g., methylsulfonyloxy, ethylsulfonyloxy, propylsulfonyloxy, butylsulfonyloxy, pentylsulfonyloxy, hexylsulfonyloxy, etc.), etc.];
(9) substituted or unsubstituted arylsulfonyloxy (e.g., p-toluenesulfonyloxy, benzenesulfonyloxy, mesitylenesulfonyloxy, etc.), etc. The number of the substituent may be one or two or more. Where the number of the substituent is two or more, the substituents may be the same or different.

Suitable examples of R¹⁸ may include hydrogen, methyl, ethyl, tert-butoxycarbonylethyl, carboxyethyl, hydroxypropyl, methoxyethyl, hydroxyethyl, dimethylaminopropyl, etc.

### (Definition of R¹⁹)

In the above-mentioned formula (B2), R¹⁹ is hydrogen or substituted or unsubstituted lower alkyl.

Examples of the “lower alkyl” of the “substituted or unsubstituted lower alkyl” for R¹⁹ may include lower alkyl similar to those exemplified for R¹ above, in which the preferred one may be (C₁₋₁₄)alkyl and more preferred one may be ethyl, propyl, etc.

Examples of the substituents for the “substituted lower alkyl” for R¹⁹ may include

(1) hydroxy;
(2) carboxy;
(3) (lower)alkoxycarbonyl [e.g., (C₁₋₆)alkoxycarbonyl (e.g., methoxycarbonyl, ethoxycarbonyl, propoxycarbonyl, butoxycarbonyl, pentyloxycarbonyl, hexyloxycarbonyl, etc.), etc.];
(4) saturated cyclic amino [e.g., 4-, 5- or 6-membered saturated cyclic amino which may further have heteroatom(s) selected from a nitrogen atom, an oxygen atom and a sulfur atom and/or oxo besides the amino nitrogen and may have substituent(s), such as azetidinyl (e.g., 3-hydroxy-1-azetidinyl, 3-amino-1-azetidinyl, etc.), morpholinyl (e.g., morpholino, etc.), etc.];
(5) (saturated cyclic amino)carbonyl [e.g., a group in which the saturated cyclic amino as exemplified in (4) above is attached to a carbonyl group (e.g., morpholinocarbonyl, etc.), etc.];
(6) (lower)alkylsulfonyloxy [e.g., (C₁₋₆)alkylsulfonyloxy (e.g., methylsulfonyloxy, ethylsulfonyloxy, propylsulfonyloxy, butylsulfonyloxy, pentylcarbonyloxy, hexylcarbonyloxy, etc.), etc.];
(7) substituted or unsubstituted amino [e.g., amino, mono- or di-(substituted or unsubstituted lower alkyl)amino (e.g., mono-(C₁₋₆)alkylamino (e.g., methylamino, ethylamino, propylamino, isopropylamino, butylamino, tert-butylamino, neopentylamino, etc.), di-(C₁₋₄)alkylamino (e.g., dimethylamino, diethylamino, ethylmethylamino, etc.), 2-hydroxyethylamino, 2-methoxyethylamino, 2-(dimethylamino)ethylamino, 2-hydroxy-1,1-dimethylethylamino, 2-hydroxy-1-(hydroxymethyl)ethylamino, (2-hydroxyethyl)methylamino, (2-methoxyethyl)methylamino, etc.), mono-(C₂₋₅)alkanoylamino (e.g., acetylamino, ethylcarbonylamino, propylcarbonylamino, isopropylcarbonylamino, butylcarbonylamino, etc.), (C₃₋₈) cycloalkylamino (e.g., cyclopropylamino, cyclobutylamino, cyclopentylamino, cyclohexylamino, etc.), etc.),
(8) substituted or unsubstituted arylsulfonyloxy (e.g., p-toluenesulfonyloxy, benzenesulfonyloxy, mesitylenesulfonyloxy, etc.);
(9) halogen (e.g., chloro, fluoro, bromo, iodo, etc.), etc. The number of the substituent may be one or two or more. Where the number of the substituent is two or more, the substituents may be the same or different.

Suitable examples of R¹⁹ may include methyl, ethyl, propyl, methoxyethyl, methoxypropyl, hydroxyethyl, ethoxycarbonylethyl, carboxyethyl, hydroxypropyl, morpholinocarbonylethyl, methylsulfonyloxypropyl, morpholinopropyl, methylaminopropyl, dimethylaminopropyl, etc.

Specific examples of the preferred compound of the present invention may be exemplified by Examples below.

In order to show the usefulness of the compound (I) of the present invention, the pharmacological test results of the representative compounds of the present invention are shown in the following.

### Test 1: Inhibition of TNF-α Production in THP-1 Cells

[I] Test Method

THP-1 cells, a human monocytic cell line, were maintained in RPMI 1640 (Sigma R8758) supplemented with penicillin (50 U/ml), streptomycin (50 μg/ml) and 10% fetal bovine serum (Moregate BioTech.) at 37° C., 5% CO₂ in a humidified incubator. Initial stock solutions of test compounds were made in DMSO. All cells, reagents and test compounds were diluted into culture media. THP-1 cells (1×10⁵ cells/well final) and lipopolysaccharide (LPS; 10 μg/mL final; Sigma L-4005, from E. coli serotype 055:B5) were added to 96 well polypropylene culture plates (Sumilon, MS-8196F5; sterile) containing test compound or 0.1% DMSO vehicle. The cell mixture was incubated for 20 hours in a humidified incubator at 37° C., 5% CO₂. The culture supernatants were harvested and TNF-α levels from LPS stimulated cells in the presence of 100 nM test compound was calculated compared with control cells stimulated in the presence of 0.1% DMSO.

[II] Test Compounds
6-{2-(2,4-Difluorophenyl)-6-[(dimethylamino)methyl]-4,5,6,7-tetrahydropyrazolo[1,5-a]pyrimidin-3-yl}-2-(2-methylphenyl)-3(2H)-pyridazinone (Example 1)
6-{2-(2,4-Difluorophenyl)-6-[(dimethylamino)methyl]pyrazolo[1,5-a]pyrimidin-3-yl}-2-(2-methylphenyl)-3(2H)-pyridazinone (Example 2)
6-[1-Ethyl-6-(4-fluorophenyl)-2,3-dihydro-1H-imidazo[1,2-b]pyrazol-7-yl]-2-(2-methylphenyl)-3(2H)-pyridazinone (Example 6)
6-[2-(4-Fluorophenyl)-6,6-bis(hydroxymethyl)-4,5,6,7-tetrahydropyrazolo[1,5-a]pyrimidin-3-yl]-2-(2-methylphenyl)pyridazin-3(2H)-one (Example 35)
6-[2-(2,4-Difluorophenyl)-6-(hydroxymethyl)-4,5,6,7-tetrahydropyrazolo[1,5-a]pyrimidin-3-yl)-2-(2-methylphenyl)pyridazin-3(2H)-one (Example 37)
6-{2-(4-Fluorophenyl)-6-[(4-methylpiperazin-1-yl)methyl]-4,5,6,7-tetrahydropyrazolo[5-a]pyrimidin-3-yl}-2-(2-methylphenyl)pyridazin-3(2H)-one dihydrochloride (Example 47)
6-{2-(2,4-difluorophenyl)-6-[(dimethylamino)methyl]-4,5,6,7-tetrahydropyrazolo[1,5-a]pyrimidin-3-yl}-2-(2-methylphenyl)-4,5-dihydropyridazin-3(2H)-one (Example 55)
N-cyclopropyl-2-(4-fluorophenyl)-3-[1-(2-methylphenyl)-6-oxo-1,6-dihydropyridazin-3-yl]-4,5,6,7-tetrahydropyrazolo[1,5-a]pyrimidine-6-carboxamide (Example 57)
6-[6,6-Difluoro-2-(4-fluorophenyl)-4,5,6,7-tetrahydropyrazolo[1,5-a]pyrimidin-3-yl]-2-(2-methylphenyl)pyridazin-3(2H)-one (Example 85)
6-{6-[(tert-Butylamino)methyl]-2-(2,4-difluorophenyl)-4,5,6,7-tetrahydropyrazolo[1,5-a]pyrimidin-3-yl}-2-(2-methylphenyl)pyridazin-3(2H)-one (Example 98)
6-[1-Acetyl-2′-(4-fluorophenyl)-4′,5′-dihydrospiro[piperidine-4,6′-pyrazolo[1,5-a]pyrimidin]-3′-yl]-2-(2-methylphenyl)pyridazin-3(2H)-one (Example 107)
6-[(5S)-2-(4-Fluorophenyl)-5-(hydroxymethyl)-4,5,6,7-tetrahydropyrazolo[1,5-a]pyrimidin-3-yl]-2-(2-methylphenyl)pyridazin-3(2H)-one (Example 123)
6-[(5S)-2-(4-Fluorophenyl)-5-(hydroxymethyl)-4,5,6,7-tetrahydropyrazolo[1,5-a]pyrimidin-3-yl]-2-(2-methylphenyl)pyridazin-3(2H)-one (Example 124)
Ethyl 3-(4-fluorophenyl)-2-[1-(2-methylphenyl)-6-oxo-1,6-dihydropyridazin-3-yl]-3-oxopropanoate (Example 125)
6-(5-Isopropyl-2-phenyl-4,5,6,7-tetrahydropyrazolo[1,5-a]pyrazin-3-yl)-2-(2-methylphenyl)pyridazin-3(2H)-one (Example 130)

[III] Test Results

TABLE 1
Inhibition of TNF-α production in THP-1 cells at 100 nM

| Test compounds | % inhibition |
|---|---|
| (Example Nos.) | of control |
| Example 1 | 88 |
| Example 2 | 98 |
| Example 6 | 80 |
| Example 35 | 90 |
| Example 37 | 94 |
| Example 47 | 95 |
| Example 55 | 98 |
| Example 57 | 97 |
| Example 85 | 88 |
| Example 98 | 86 |
| Example 107 | 90 |
| Example 123 | 94 |
| Example 124 | 94 |
| Example 125 | 83 |
| Example 130 | 70 |

Test 2: Inhibition of Hind Paw Swelling in Adjuvant-Induced Arthritis Rats

[I] Test Method

Arthritis was induced by injection of 0.5 mg of Mycobacterium tuberculosis (Difco Laboratories, Detroit, Mich.) in 50 μL of liquid paraffin into the right hind footpad of female Lewis rats aged 7 weeks (day 0). Normal untreated rats were used as negative controls. Animals were randomized and grouped (n≧5) for drug treatment based on an increase of left hind paw volume and body weight on day 15. Test compounds were suspended in vehicle (0.5% methylcellulose) and orally administered once a day from days 15 to 24. The volume of the left hind paw was measured on day 25 by a water displacement method using a plethymometer for rats (MK-550; Muromachi Kikai Co., Ltd., Tokyo, Japan).

[II] Test Compounds
6-[2-(4-Fluorophenyl)-6-(hydroxymethyl)-4,5,6,7-tetrahydropyrazolo[1,5-a]pyrimidin-3-yl]-2-(2-methylphenyl)-3(2H)-pyridazinone (Example 3)
6-[2-(4-Fluorophenyl)-6-hydroxy-4,5,6,7-tetrahydropyrazolo[1,5-a]pyrimidin-3-yl]-2-(2-methylphenyl)-3(2H)-pyridazinone (Example 18)
6-[2-(2,4-Difluorophenyl)-6-(hydroxymethyl)-4,5,6,7-tetrahydropyrazolo[1,5-a]pyrimidin-3-yl]-2-(2-methylphenyl)pyridazin-3(2H)-one (Example 37)
6-[2′-(4-Fluorophenyl)-2,3,4′,5,5′,6-hexahydrospiro[pyran-4,6′-pyrazolo[1,5-a]pyrimidin]-3′-yl]-2-(2-methylphenyl)pyridazin-3(2H)-one (Example 63)
6-[2′-(4-Fluorophenyl)-4′,5′-dihydrospiro[1,3-dioxolane-2,6′-pyrazolo[1,5-a]pyrimidin]-3′-yl]-2-(2-methylphenyl)pyridazin-3(2H)-one (Example 86)
6-[(6R)-2-(4-Fluorophenyl)-6-hydroxy-4,5,6,7-tetrahydropyrazolo[1,5-a]pyrimidin-3-yl]-2-(2-methylphenyl)pyridazin-3(2H)-one (Example 100)
6-[(5S)-2-(4-fluorophenyl)-5-(hydroxymethyl)-4,5,6,7-tetrahydropyrazolo[1,5-a]pyrimidin-3-yl)-2-(2-methylphenyl)pyridazin-3(2H)-one (Example 123)
6-[(5S)-2-(4-fluorophenyl)-5-(hydroxymethyl)-4,5,6,7-tetrahydropyrazolo[1,5-a]pyrimidin-3-yl]-2-(2-methylphenyl)pyridazin-3(2H)-one (Example 124)
6-[2-(4-Fluorophenyl)-6,6-dimethyl-4,5,6,7-teterahydropyrazolo[1,5-a]pyrimidin-3-yl]-2-(2-methylphenyl)pyridazin-3(2H)-one (Example 132)

[III] Test Results

TABLE 2
Inhibition of hind paw swelling in adjuvant-induced arthritis rats

| Test | Dose | % inhibition of |
|---|---|---|
| compounds | (mg/kg) | vehicle-treated rats |
| Example 1 | 0.3 | 51.2 |
| Example 3 | 1 | 56.4 |
| Example 18 | 1 | 46.1 |
| Example 37 | 1 | 63.6 |
| Example 63 | 1 | 39.5 |
| Example 86 | 1 | 48.1 |
| Example 100 | 0.5 | 40.2 |
| Example 123 | 1 | 62.3 |
| Example 124 | 1 | 50.6 |
| Example 132 | 1 | 60.4 |

The compound (I) and a salt thereof of the present invention are useful as inhibitors of cytokines' production or their transduction, and through inhibiting the p38α MAPK they possess pharmacological actions such as analgesic action, anti-inflammatory, anti arthritis mutilans action, or the like, and for the prevention and/or the treatment of pain, rheumatoid arthritis, other conditions associated with inflammation, Crohn's disease, inflammatory bowel disease, psoriasis, or the like.

The pharmaceutical composition of the present invention can be used in the form of a pharmaceutical preparation, for example, in a solid, semisolid or liquid form, which contains the compound (I) or a pharmaceutically acceptable salt thereof as an active ingredient in admixture with an organic or inorganic carrier or excipient suitable for rectal, pulmonary (nasal or buccal inhalation), nasal, ocular, external (topical), oral or parenteral (including subcutaneous, intravenous and intramuscular) administrations or insufflation. The active ingredient may be compounded, for example, with the usual non-toxic, pharmaceutically acceptable carriers for tablets, pellets, troches, capsules, suppositories, creams, ointments, aerosols, powders for insufflation, solutions, emulsions, suspensions, and any other form suitable for use. In addition, auxiliary, stabilizing agents, thickening agents, coloring agents and perfumes may be used where necessary. The compound (I) or a pharmaceutically acceptable salt thereof is included in a pharmaceutical composition in an amount sufficient to produce the desired aforesaid pharmaceutical effect upon the process or condition of diseases.

For applying the composition to a mammal (e.g., human being, mouse, rat, swine, dog, cat, horse, bovine, etc., especially human being), it is preferable to apply the composition by intravenous, intramuscular, pulmonary or oral administration, or insufflation. While the dosage of therapeutically effective amount of the compound (I) varies depending on the age and condition of each individual patient to be treated, in the case of intravenous administration, a daily dose of 0.01-100 mg of the compound (I) per kg weight of a mammal, in the case of intramuscular administration, a daily dose of 0.1-100 mg of the compound (I) per kg weight of a mammal, and in case of oral administration, a daily dose of 0.5-100 mg of the compound (I) per kg weight of a mammal is generally given for the prevention and/or treatment of the aforesaid diseases.

Hereinafter the reactions for preparing the compound [I] of the present invention are explained in more detail with referring to the Preparations and Examples. However, the Preparations and Examples are given only for the purpose of illustration of the present invention, and the invention should not be restricted by the Preparations and Examples in any way.

The abbreviations, symbols and terms used in the Preparations and Examples have the following meanings.

AcOH acetic acid

CDCl₃ chloroform-d

CHCl₃ chloroform

CH₂Cl₂ dichloromethane

CH₃CN acetonitrile

EtOAc or AcOEt ethyl acetate

MeOH methanol

EtOH ethanol

PrOH propanol

i-PrOH or IPA isopropyl alcohol

BUOH butanol t-(or tert-)BuOH t-(or tert-)butanol

DME 1,2-dimethoxyethane

DMF N,N-dimethylformamide

DMSO dimethyl sulfoxide

Et₃N triethylamine

IPE diisopropyl ether

TFA trifluoroacetic acid

THF tetrahydrofuran

HOBt or HOBT 1-hydroxybenzotriazole

EDCI or WSCD 1-ethyl-3-[3′-(dimethylamino)propyl]carbodiimide

Pd/C palladium on carbon

MCPBA or mCPBA 3-chloroperoxybenzoic acid

min minute(s)

hr or h hour(s)

rt room temperature

conc. concentrated

aq aqueous (ex. aq NaHCO₃ solution)

HCl hydrochloric acid

CuBr₂ copper (II) bromide

Na₂CO₃ sodium carbonate

NaOH sodium hydroxide

Na₂SO₄ sodium sulfate

### Preparation 1

To a solution of 3-chloro-6-methylpyridazine (51 g) and ethyl 4-fluorobenzoate (66.7 g) in THF (200 ml) was added dropwise lithium bis(trimethylsilyl)amide (793 ml, 1.0 M in THF) over the period of 30 min while maintaining the temperature below 15° C. After stirring for 30 min at room temperature, the mixture was recooled in an ice bath, and neutralized by addition of cold water (250 ml) and 6 N HCl (175 ml). A solid was separated from the mixture and collected to give 2-(6-chloro-3-pyridazinyl)-1-(4-fluorophenyl)ethanone (36.6 g) as the first crop. The organic layer was separated from the mother liquor and washed with brine (150 ml, twice), dried over Na₂SO₄, filtered and concentrated to form a suspension. This suspension was dissolved under reflux. To the solution was added hexane (600 ml) and the resulted suspension was aged for 1 hour with stirring at room temperature. The resulted solid was collected and washed with hexane (200 ml) to afford 2-(6-chloro-3-pyridazinyl)-1-(4-fluorophenyl)ethanone (51.3 g) as the second crop.

Mass ESI (+) 251 (M+1)

¹H-NMR (300 MHz, DMSO-d₆) δ 4.85 (2H, s), 7.42 (2H, t, J=9 Hz), 7.78 (1H, d, J=8.7 Hz), 7.93 (1H, d, J=8.7 Hz), 8.13-8.22 (2H, m)

### Preparation 2

A mixture of 2-(6-chloro-3-pyridazinyl)-1-(4-fluorophenyl)ethanone (30.0 g) and sodium acetate (19.6 g) in AcOH (240 ml) was stirred for 3 hours at 135° C. After cooling to room temperature, cold water (400 ml) was added to this mixture. A solid separated from the mixture was collected, washed with water and dried in vacuo to give 6-[2-(4-fluorophenyl)-2-oxoethyl]-3(2H)-pyridazinone (17 g) as a gray solid.

Mass ESI (+) 233 (M+1)

¹H-NMR (300 MHz, DMSO-d₆) δ 4.43 (2H, s), 6.87 (1H, d, J=10 Hz), 7.36-7.43 (3H, m), 8.09-8.14 (2H, m)

### Preparation 3

A mixture of 6-(2-(4-fluorophenyl)-2-oxoethyl]-3(2H)-pyridazinone (4.8 g), ethylene glycol (9.6 ml) and toluenesulfonic acid hydrate (393 mg) in toluene (96 ml) was refluxed for 6 h with azeotropic removal of water.

After concentration, the residue was partitioned between EtOAc and saturated aqueous NaHCO₃. The organic layer was washed with brine, dried over Na₂SO₄, filtered and evaporated in vacuo to give a solid. The solid was triturated with hexane, collected and dried in vacuo to afford 6-{[2-(4-fluorophenyl)-1,3-dioxolan-2-yl]methyl}—3(2H)-pyridazinone (3.04 g) as a white solid.

¹H-NMR (200 MHz, DMSO-d₆) δ 3.10 (2H, s), 3.67-3.74 (2H, m), 3.89-3.97 (2H, m), 6.76 (1H, d, J=9.8 Hz), 7.11-7.20 (2H, m), 7.28 (1H, d, J=9.8 Hz), 7.33-7.40 (2H, m), 12.76 (1H, s)

### Preparation 4

A mixture of 6-{[2-(4-fluorophenyl)-1,3-dioxolan-2-yl]methyl}-3(2H)-pyridazinone (2.0 g), 2-methylbenzeneboronic acid (2.46 g), copper (II) acetate (263 mg) and pyridine (2.93 ml) in DMF (30 ml) was stirred for 14 hours at room temperature. The mixture was partitioned between EtOAc and H₂O. The separated organic layer was washed with brine, dried over Na₂SO₄, filtered and evaporated in vacuo. The residue was purified by column chromatography on SiO₂ (eluent; 1% to 8% methanol in dichloromethane) to give 6-{[2-(4-fluorophenyl)-1,3-dioxolan-2-yl]methyl}-2-(2-methylphenyl)-3(2H)-pyridazinone (2.17 g) as an amorphous solid.

¹H-NMR (200 MHz, DMSO-d₆) δ 1.83 (3H, s), 3.16 (2H, s), 3.70-3.34 (2H, m), 3.89-4.04 (2H, m), 6.95-7.07 (2H, m), 7.09-7.23 (2H, m), 7.24-7.41 (5H, m), 7.46 (1H, d, J=9.5 Hz)

### Preparation 5

To a solution of 6-{[2-(4-fluorophenyl)-1,3-dioxolan-2-yl]methyl}-2-(2-methylphenyl)-3(2H)-pyridazinone (2.16 g) in THF (20 ml) was added conc. HCl (2 ml) at room temperature. After stirring for 14 hours, the mixture was concentrated and partitioned between EtOAc and water. The organic layer was washed with 3% aqueous NaHCO₃ and brine, dried over Na₂SO₄, filtered and concentrated in vacuo. The residue was purified by column chromatography on SiO₂ (eluent; 30% to 50% EtOAc in dichloromethane) to give 6-[2-(4-fluorophenyl)-2-oxoethyl]-2-(2-methylphenyl)-3(2H)-pyridazinone (1.64 g) as a pale yellow waxy solid.

¹H-NMR (200 MHz, DMSO-d₆) δ 2.01 (3H, s), 4.51 (2H, s), 7.08 (1H, d, J=9.6 Hz), 7.20-7.47 (6H, m), 7.53 (1H, d, J=9.6 Hz), 8.05-8.18 (2H, m)

### Preparation 6

To a solution of 6-[2-(4-fluorophenyl)-2-oxoethyl]-2-(2-methylphenyl)-3(2H)-pyridazinone (500 mg) in AcOH (4 ml) was added pyridinium tribromide (595 mg) portionwise at room temperature. After 3 h, the mixture was partitioned between EtOAc (8 ml) and water (16 ml). The separated organic layer was washed with water, 3% aqueous Na₂S₂O₃, 3% aqueous NaHCO₃ (two times) and brine, dried over Na₂SO₄, filtered and concentrated in vacuo to give 6-[1-bromo-2-(4-fluorophenyl)-2-oxoethyl]-2-(2-methylphenyl)-3(2H)-pyridazinone (566 mg) as a pale yellow solid.

¹H-NMR (200 MHz, DMSO-d₆) δ 1.89 (3H, s), 7.08 (1H, s), 7.15-7.48 (7H, m), 7.80 (1H, d, J=9.7 Hz), 7.08-8.20 (2H, m)

### Preparation 7

2-(6-Chloro-3-pyridazinyl)-1-(2,4-difluorophenyl)ethanone was obtained according to a similar manner to Preparation 1.

¹H-NMR (200 MHz, DMSO-d₆) δ 4.74 (1.6H, d, J=2.5 Hz), 6.25 (0.2H, s), 7.18-7.37 (1H, m), 7.39-7.56 (1H, m), 7.75-7.87 (1.2H, m), 7.88-8.11 (2H, m)

### Preparation 8

6-[2-(2,4-Difluorophenyl)-2-oxoethyl]-3(2H)-pyridazinone was obtained according to a similar manner to Preparation 2.

¹H-NMR (200 MHz, DMSO-d₆) δ 4.32 (2H, d, J=3.0 Hz), 6.86 (1H, dd, J=1.5, 10.0 Hz), 7.27 (1H, dt, J=2.5, 8.0 Hz), 7.38 (1H, d, J=10.0 Hz), 7.40-7.53 (1H, m), 7.91-8.08 (1H, m), 12.91 (1H, brs)

### Preparation 9

6-{[2-(2,4-Difluorophenyl)-1,3-dioxolan-2-yl]methyl}-3(2H)-pyridazinone was obtained according to a similar manner to Preparation 3.

¹H-NMR (200 MHz, DMSO-d₆) δ 3.19 (2H, s), 3.72-3.87 (2H, m), 3.88-4.02 (2H, m), 6.76 (1H, d, J=10.0 Hz), 7.01 (1H, dt, J=2.5, 8.5 Hz), 7.17-7.42 (3H, m), 12.73 (1H, brs)

### Preparation 10

A mixture of 6-{[2-(2,4-difluorophenyl)-1,3-dioxolan-2-yl]methyl}-3(2H)-pyridazinone (8.00 g), 2-methylbenzeneboronic acid (7.39 g), copper (II) acetate (988 mg) and pyridine (10.75 g) in DMF (80 ml) was stirred at room temperature for 2 days. The mixture was partitioned between EtOAc (120 ml) and 3% aqueous NaHCO₃ (160 ml). The organic layer was washed with 3% aqueous citric acid (×2), 0.5 N NaOH (×2) and brine, dried over Na₂SO₄, filtered and evaporated in vacuo. The residue was purified by column chromatograph on SiO₂ (eluent; EtOAc/Hex (w/w)=1/1 to 2/1) to give 6-{[2-(2,4-difluorophenyl)-1,3-dioxolan-2-yl]methyl}-2-(2-methylphenyl)-3(2H)-pyridazinone (8.29 g) as a waxy solid.

¹H-NMR (200 MHz, DMSO-d₆) δ 1.81 (3H, s), 3.24 (2H, s), 3.74-3.90 (2H, m), 3.93-4.08 (2H, m), 6.92-7.09 (3H, m), 7.14-7.39 (5H, m), 7.47 (1H, d, J=9.6 Hz)

### Preparation 11

6-[2-(2,4-Difluorophenyl)-2-oxoethyl]-2-(2-methylphenyl)-3(2H)-pyridazinone was obtained according to a similar manner to Preparation 5.

¹H-NMR (200 MHz, DMSO-d₆) δ 2.00 (3H, s), 4-0.40 (2H, d, J=2.6 Hz), 7.08 (1H, d, J=9.6 Hz), 7.19-7.58 (7H, m), 7.94-8.09 (1H, m)

### Preparation 12

6-[1-Bromo-2-(2,4-difluorophenyl)-2-oxoethyl]-2-(2 methylphenyl)-3(2H)-pyridazinone was obtained according to a similar manner to Preparation 6.

¹H-NMR (200 MHz, DMSO-d₆) δ 1.81 (3H, s), 6.76 (1H, s), 6.99-7.58 (7H, m), 7.80 (1H, d, J=9.7 Hz), 7.98-8.14 (1H, m).

### Preparation 13

A mixture of 4-methyl-4-phenylthiosemicarbazide (544 mg), 3-(dimethylaminomethyl)azetidine dihydrochloride (561 mg) and 1,8-diazabicyclo[5.4.0]undec-7-ene (0.94 ml) in acetonitrile (2 mL) was stirred at 90° C. for 3 hours. The mixture was cooled to room temperature. To the mixture was added water (20 mL), and the mixture was washed with ether (20 mL). The aqueous layer was extracted with chloroform (40 mL×2). The extracts were combined, dried over anhydrous magnesium sulfate and concentrated under reduced pressure. The oily residue was crystallized from diisopropyl ether to give 3-[(dimethylamino)methyl]-1-azetidinecarbothiohydrazide (248 mg) as gray powder.

¹H-NMR (500 MHz, CDCl₃) δ 2.22 (s, 6H), 2.52 (d, 2H, J=7.5 Hz), 2.80-2.85 (m, 1H), 3.78 (dd, 2H, J=5.5 Hz, 10.0 Hz), 4.20 (t, 2H, J=8.5 Hz), 6.39 (brs, 1H)

### Preparation 14

To a solution of 2-hydrazinoethanol (0.88 mL) in ethanol (8 mL) was added dropwise a solution of ethyl 3-isothiocyanatopropionate (1.42 mL) in ethanol (8 mL) at room temperature. The mixture was stirred at room temperature overnight. The solvent was removed under reduced pressure. The residue was purified by flash column chromatography (gradient elution: methanol/chloroform (w/w)=0% to 6%) to give ethyl 3-({[1-(2-hydroxyethyl)hydrazino]carbonothioyl}amino)propanoate (2.40 g) as colorless oil.

¹H-NMR (500 MHz, CDCl₃) δ 1.27 (3H, t, J=7.5 Hz), 1.62 (1H, brs), 2.36 (1H, brs), 2.66 (2H, t, J=5.9 Hz), 3.90 (2H, q, J=6.0 Hz), 4.02-4.05 (2H, m), 4.08 (2H, s), 4.17 (2H, q, J=7.3 Hz), 4.30 (2H, t, J=5.0 Hz), 8.36 (1H, brs).

### Preparation 15

6-[5-(Ethylamino)-3-(4-fluorophenyl)-1-(2-hydroxyethyl)-1H-pyrazol-4-yl]-2-(2-methylphenyl)-3(2H)-pyridazinone was obtained according to a similar manner to Example 1 mentioned below.

Mass ESI (+) 434 (M+1)

¹H-NMR (500 MHz, CDCl₃) δ 1.03 (3H, t, J=6.7 Hz), 2.22 (3H, s), 3.04-3.09 (2H, m), 3.83 (1H, brs), 4.01-4.03 (2H, m), 4.18 (2H, t, J=4.7 Hz), 5.15 (1H, brs), 6.87 (1H, d, J=9.8 Hz), 7.02 (1H, d, J=9.8 Hz), 7.13 (2H, t, J=8.2 Hz), 7.28-7.39 (4H, m), 7.48 (2H, dd, J=5.6, 8.8 Hz)

### Preparation 16

6-[5-(Ethylamino)-3-(4-fluorophenyl)-1-(3-hydroxypropyl)-1H-pyrazol-4-yl]-2-(2-methylphenyl)-3(2H)-pyridazinone was obtained according to a similar manner to Example 1 mentioned below.

Mass ESI (+) 448 (M+1)

¹H-NMR (500 MHz, CDCl₃) δ 1.04 (3H, t, J=7.8 Hz), 2.02-2.06 (2H, m), 2.23 (3H, s), 3.04-3.10 (2H, m), 3.55-3.63 (3H, m), 4.24 (2H, t, J=6.8 Hz), 5.21 (1H, brs), 7.14 (2H, t, J=8.4 Hz), 7.29-7.40 (4H, m), 7.48 (2H, dd, J=5.5, 8.7 Hz)

### Preparation 17

Ethyl 3-({3-(4-fluorophenyl)-1-(2-hydroxyethyl)-4-[1-(2-methylphenyl)-6-oxo-1,6-dihydro-3-pyridazinyl]-1H-1-pyrazol-5-yl}amino)propanoate was obtained according to a similar manner to Example 1 mentioned below.

Mass ESI (+) 506 (M+1)

¹H-NMR (500 MHz, CDCl₃) δ 1.19 (3H, t, J=7.0 Hz), 2.24 (3H, s), 2.36 (2H, t, J=6.5 Hz), 3.31 (2H, dd, J=6.4, 12.7 Hz), 3.65 (1H, t, J=6.0 Hz), 4.01-4.08 (4H, m), 4.20 (2H, t, J=4.6 Hz), 5.18 (1H, t, J=7.1 Hz), 6.89 (1H, d, J=9.6 Hz), 7.01 (1H, d, J=9.5 Hz), 7.13 (2H, dd, J=8.8, 8.8 Hz), 7.36-7.70 (4H, m), 7.47 (2H, dd, J=5.5, 8.7 Hz)

### Preparation 18

tert-Butyl 3-[{3-(4-fluorophenyl)-4-[1-(2-methylphenyl)-6-oxo-1,6-dihydro-3-pyridazinyl]-1H-pyrazol-5-yl}(3-hydroxypropyl)amino]propanoate was obtained according to a similar manner to Example 1 mentioned below.

¹H-NMR (500 MHz, CDCl₃) δ 1.42 (6H, s), 1.45 (3H, s), 1.74-1.79 (2H, m), 2.10 (3H, s), 2.41 (2H, t, J=7.5 Hz), 3.31 (2H, t, J=6.9 Hz), 3.42 (2H, t, J=7.5 Hz), 3.61 (2H, t, J=5.3 Hz), 6.98 (1H, d, J=9.6 Hz), 7.03 (2H, dd, J=8.6, 8.6 Hz), 7.17 (1H, d, J=6.4 Hz), 7.28-7.42 (6H, m)

### Preparation 19

N-Ethyl-1-(3-hydroxypropyl)hydrazinecarbothioamide was obtained according to a similar manner to Preparation 14.

¹H-NMR (CDCl₃) δ 1.23 (3H, t, J=7.3 Hz), 1.65-1.66 (1H, m), 1.82-1.86 (2H, m), 3.55-3.64 (5H, m), 3.73 (2H, s), 4.28 (2H, t, J=5.9 Hz), 7.81 (1H, brs)

### Preparation 20

6-[1-Bromo-2-(2,4-difluorophenyl)-2-oxoethyl]-3(2H)-pyridazinone was obtained according to a similar manner to

### Preparation 6.

¹H-NMR (200 MHz, DMSO-d₆) δ 6.69 (1H, s), 6.93-7.11 (1H, m), 7.29 (1H, dt, J=2.7, 8.7 Hz), 7.38-7.53 (1H, m), 7.64 (1H, d, J=9.9 Hz), 8.08 (1H, dt, J=6.6, 8.9 Hz), 13.08-13.27 (1H, m)

### Preparation 21

To a suspension of LiAlH₄ (543 mg) in THF (20 mL) was added dropwise a solution of 4-(hydroxymethyl)tetrahydro-2H-thiopyran-4-carbonitrile (1.50 g) in THF (20 mL) at 0° C. The mixture was stirred for 1 h at the same temperature and the reaction was quenched by slow addition of H₂O (0.5 mL), 10% aqueous NaOH (0.5 mL) and H₂O (0.5 mL×3) with ice cooling. After 10 min with stirring, the insoluble materials were filtered off and the filter cake was washed with EtOAc. The filtrate was dried over MgSO₄, filtered and concentrated in vacuo to give [4-(aminomethyl)tetrahydro-2H-thiopyran-4-yl]methanol (1.30 g) as a pale yellow oil.

Mass ESI (+) 162 (M+1)

### Preparation 22

To a mixture of [4-(aminomethyl)tetrahydro-2H-thiopyran-4-yl]methanol (1.20 g) in CH₂Cl₂ (20 mL) and aqueous NaHCO₃ (1.25 g in 10 mL of H₂O) was added O-phenyl chlorothiocarbonate (1.54 g) portionwise and the mixture was stirred for 30 min vigorously at room temperature. The organic layer was separated and the aqueous solution was extracted with CHCl₃. The combined organic layer was washed with brine, dried over MgSO₄, filtered and concentrated in vacuo. The residue was purified by column chromatography (eluent: Hex/EtOAc=1/1) to give O-phenyl {[4-(hydroxymethyl)tetrahydro-2H-thiopyran-4-yl]methyl}thiocarbamate (740 mg) as a colorless oil.

Mass ESI (+) 320 (M+Na)

### Preparation 23

To a solution of O-phenyl {[4-(hydroxymethyl)tetrahydro-2H-thiopyran-4-yl]methyl}thiocarbamate (740 mg) in i-PrOH (10 mL) was added hydrazine monohydrate (1.25 g) and the mixture was stirred for 3 h at room temperature. The whole mixture was diluted with brine and CHCl₃. The aqueous layer was extracted with CHCl₃. The combined organic layer was washed with 0.5 M aqueous NaOH and brine, dried over MgSO₄, filtered and concentrated to give N-{[4-(hydroxymethyl)tetrahydro-2H-thiopyran-4-yl]methyl}hydrazinecarbothioamide (300 mg) as a white solid.

Mass ESI (+) 258 (M+Na)

### Preparation 24

6-(5-{[(2S)-2-(Benzyloxy)-3-hydroxypropyl]amino}-3-(4-fluorophenyl)-1H-pyrazol-4-yl]-2-(2-methylphenyl)pyridazin-3(2H)-one was obtained according to a similar manner to Example 1 mentioned below.

Mass ESI (+) 526 (M+1)

¹H-NMR (CDCl₃) δ 2.20 (3H, s), 3.40-3.65 (5H, m), 4.49 (2H, s), 6.00 (1H, br), 6.82 (1H, d, J=9.9 Hz), 6.97 (1H, d, J=9.9 Hz), 7.15-7.25 (4H, m), 7.25-7.36 (7H, m), 7.41-7.48 (2H, m)

### Preparation 25

6-[5-{[(2S)-2-(Benzyloxy)-3-hydroxypropyl]amino}-3-(2,4-difluorophenyl)-1H-pyrazol-4-yl]-2-(2-methylphenyl)pyridazin-3(2H)-one was obtained according to a similar manner to Example 1 mentioned below.

Mass ESI (+) 544 (M+1)

¹H-NMR (CDCl₃) δ 2.19 (3H, s), 3.39-3.67 (5H, m), 4.45-4.58 (2H, m), 6.12 (1H, br), 6.85 (1H, d, J=9.9 Hz), 6.95-7.06 (2H, m), 6.95 (1H, dd, J=1.4, 9.9 Hz), 7.20-7.26 (2H, m), 7.26-7.37 (7H, m), 7.41-7.51 (1H, m)

### Preparation 26

6-[5-{[(2R)-2-(Benzyloxy)-3-hydroxypropyl]amino}-3-[(4-fluorophenyl)-1H-pyrazol-4-yl]-2-(2-methylphenyl)pyridazin-3(2H)-one was obtained according to a similar manner to Example 1 mentioned below.

Mass ESI (+) 526 (M+1)

¹H-NMR (CDCl₃) δ 2.20 (3H, s), 3.41-3.64 (5H, m), 4.49 (2H, s), 6.00 (1H, t, J=6.2 Hz), 6.81 (1H, d, J=9.9 Hz), 6.97 (1H, d, J=9.9 Hz), 7.16-7.24 (4H, m), 7.25-7.36 (7H, m), 7.42-7.48 (2H, m)

### Preparation 27

6-[5-{[(2R)-2-(Benzyloxy)-3-hydroxypropyl]amino}-3-(2,4-difluorophenyl)-1H-pyrazol-4-yl]-2-(2-methylphenyl)pyridazin-3(2H)-one was obtained according to a similar manner to Example 1 mentioned below.

Mass ESI (+) 544 (M+1)

¹H-NMR (CDCl₃) δ 2.18 (3H, s), 3.39-3.67 (5H, m), 4.42-4.53 (2H, m), 6.08 (1H, br), 6.85 (1H, d, J=10.1 Hz), 6.93-7.03 (2H, m), 6.94 (1H, dd, J=1.4, 10.1 Hz), 7.18-7.23 (2H, m), 7.24-7.35 (7H, m), 7.39-7.47 (1H, m)

### Preparation 28

6-[3-(2,4-Difluorophenyl)-5-{[(2S)-2-(2,2-dimethylpropoxy)-3-hydroxypropyl]amino}-1H-pyrazol-4-yl]-2-(2-methylphenyl)pyridazin-3(2H)-one was obtained according to a similar manner to Example 1 mentioned below.

Mass ESI (+) 544 (M+1)

¹H-NMR (CDCl₃) δ 2.18 (3H, s), 3.39-3.67 (5H, m), 4.42-4.53 (2H, m), 6.08 (1H, br), 6.85 (1H, d, J=10.1 Hz), 6.93-7.03 (2H, m), 6.94 (1H, dd, J=1.4, 10.1 Hz), 7.18-7.23 (2H, m), 7.24-7.35 (7H, m), 7.39-7.47 (1H, m)

### Preparation 29

Ethyl 3-({3-(2,4-difluorophenyl)-4-[1-(2-methylphenyl)-6-oxo-1,6-dihydropyridazin-3-yl]-1H-pyrazol-5-yl}amino)-2-(hydroxymethyl)propanoate was obtained according to a similar manner to Example 1 mentioned below.

¹H-NMR (CDCl₃) δ 1.11 (3H, d, J=6.9 Hz), 2.20 (3H, s), 2.66-2.70 (1H, m), 3.65-3.68 (2H, m), 3.76-3.82 (2H, m), 3.92-4.03 (2H, m), 6.03 (1H, brs), 6.85 (1H, d, J=9.2 Hz), 6.92-7.01 (3H, m), 7.29-7.43 (5H, m).

### Preparation 30

Ethyl 3-({3-(4-fluorophenyl)-4-[1-(2-methylphenyl)-6-oxo-1,6-dihydropyridazin-3-yl]-1H-pyrazol-5-yl}amino)-2-(hydroxymethyl)propanoate was obtained according to a similar manner to Example 1 mentioned below.

¹H-NMR (CDCl₃) δ 1.12 (3H, d, J=7.2 Hz), 2.22 (3H, s), 2.69-2.73 (1H, m), 3.66-3.72 (2H, m), 3.77-3.83 (2H, m), 3.91-4.04 (2H, m), 5.98 (1H, brs), 6.82 (1H, d, J=10.1 Hz), 6.97 (1H, d, J=10.1 Hz), 7.20 (2H, dd, J=8.6, 8.6 Hz), 7.34-7.38 (4H, m), 7.45 (2H, dd, J=5.4, 8.5 Hz).

### Preparation 31

Ethyl 3-({3-(2,4-difluorophenyl)-4-[1-(2-methylphenyl)-6-oxo-1,6-dihydropyridazin-3-yl]-1H-pyrazol-5-yl}amino)butanoate was obtained according to a similar manner to Example 1 mentioned below.

¹H-NMR (CDCl₃) δ 1.18-1.23 (6H, m), 2.22 (3H, s), 2.41-2.46 (1H, m), 2.59-2.64 (1H, m), 3.98-4.02 (1H, m), 4.10 (2H, q, J=7.3 Hz), 6.07 (1H, brs), 6.86 (1H, d, J=9.2 Hz), 6.95-7.06 (3H, m), 7.30-7.38 (4H, m), 7.50 (1H, dd, 7.8, 16.5 Hz)

### Preparation 32

6-[5-{[3-tert-Butoxy-2-(hydroxymethyl)propyl]amino}-3-(4-fluorophenyl)-1H-pyrazol-4-yl]-2-(2-methylphenyl)pyridazin-3(2H)-one was obtained according to a similar manner to Example 1 mentioned below.

¹H-NMR (CDCl₃) δ 1.11 (9H, s), 1.86-1.90 (1H, m), 2.20 (3H, s), 3.36 (2H, d, J=5.9 Hz), 3.40 (2H, dd, J=6.4, 6.4 Hz), 3.61 (2H, ddd, J=4.1, 11.0, 28.0 Hz), 5.98 (1H, brs), 6.81 (1H, d, J=9.7 Hz), 6.98 (1H, d, J=10.2 Hz), 7.17 (2H, dd, J=8.7, 8.7 Hz), 7.34-7.38 (4H, m), 7.46 (2H, dd, J=5.0, 8.2 Hz)

### Preparation 33

6-[3-(2,4-Difluorophenyl)-5-({[1-(hydroxymethyl)cyclopropyl]methyl}amino)-1H-pyrazol-4-yl]-2-(2-methylphenyl)pyridazin-3(2H)-one was obtained according to a similar manner to Example 1 mentioned below.

Mass ESI (+) 464 (M+1)

¹H-NMR (CDCl₃) δ 0.33-0.38 (4H, m), 2.22 (3H, s), 3.22 (2H, s), 3.35 (2H, brs), 6.24 (1H, brs), 6.86 (1H, d, J=9.5 Hz), 6.95-7.03 (3H, m), 7.33-7.39 (4H, m), 7.46 (1H, dd, J=8.2, 14.5 Hz)

### Preparation 34

6-[3-(4-Fluorophenyl)-5-({[1-(hydroxymethyl)cyclopropyl]methyl}amino)-1H-pyrazol-4-yl]-2-(2-methylphenyl)pyridazin-3(2H)-one was obtained according to a similar manner to Example 1 mentioned below.

Mass ESI (+) 446 (M+1)

¹H-NMR (CDCl₃) δ 0.33-0.38 (4H, m), 2.24 (3H, s), 3.23 (2H, d, J=5.9 Hz), 3.32 (2H, brs), 6.11 (1H, brs), 6.83 (1H, d, J=9.6 Hz), 6.99 (1H, d, J=9.6 Hz), 7.18 (2H, dd, J=8.5, 8.5 Hz), 7.33-7.39 (4H, m), 7.45 (2H, dd, J=5.1, 8.8 Hz)

### Preparation 35

6-[5-({[1-(Hydroxymethyl)cyclopropyl]methyl}amino)-3-(3-methylphenyl)-1H-pyrazol-4-yl]-2-(2-methylphenyl)pyridazin-3(2H)-one was obtained according to a similar manner to Example 1 mentioned below.

Mass ESI (+) 442 (M+1)

¹H-NMR (CDCl₃) δ 0.33-0.37 (4H, m), 2.24 (3H, s), 2.42 (3H, s), 3.24 (2H, brs), 3.31 (2H, brs), 6.09 (1H, brs), 6.81 (1H, d, J=10.2 Hz), 7.05 (1H, d, J=10.5 Hz), 7.24-7.40 (8H, m)

### Preparation 36

6-[3-(2,4-Difluorophenyl)-5-({[1-(hydroxymethyl)cyclobutyl]methyl}amino)-1H-pyrazol-4-yl]-2-(2-methylphenyl)pyridazin-3(2H)-one was obtained according to a similar manner to Example 1 mentioned below.

Mass ESI (+) 478 (M+1)

¹H-NMR (CDCl₃) δ 1.56-1.69 (4H, m), 1.82-1.88 (2H, m), 2.22 (3H, s), 3.36 (2H, d, J=6.5 Hz), 3.47 (2H, s), 6.24 (1H, brs), 6.85 (1H, d, J=10.1 Hz), 6.94-7.05 (3H, m), 7.33-7.40 (4H, m), 7.46 (1H, dd, J=8.2, 14.7 Hz)

### Preparation 37

6-[3-(4-Fluorophenyl)-5-([1-(hydroxymethyl)cyclobutyl]methyl)amino)-1H-pyrazol-4-yl]-2-(2-methylphenyl)pyridazin-3(2H)-one was obtained according to a similar manner to Example 1 mentioned below.

Mass ESI (+) 460 (M+1)

¹H-NMR (CDCl₃) δ 1.55-1.70 (4H, m), 1.83-1.89 (2H, m), 2.23 (3H, s), 3.37 (2H, d, J=6.5 Hz), 3.45 (2H, s), 6.11 (1H, brs), 6.82 (1H, d, J=10.2 Hz), 6.99 (1H, d, J=9.9 Hz), 7.19 (2H, dd, J=8.3, 8.3 Hz), 7.34-7.39 (4H, m), 7.45 (2H, dd, J=5.0, 8.7 Hz)

### Preparation 38

6-[3-(2,4-Difluorophenyl)-5-({[3-(hydroxymethyl)-1-isopropylazetidin-3-yl]methyl}amino)-1H-pyrazol-4-yl)-2-(2-methylphenyl)pyridazin-3(2H)-one was obtained according to a similar manner to Example 1 and Example 123, successively, mentioned below.

¹H-NMR (CDCl₃) δ 0.92 (6H, d, J=6.2 Hz), 2.20 (3H, s), 2.39-2.45 (1H, m), 2.98 (2H, d, J=8.2 Hz), 3.01 (2H, d, J=8.4 Hz), 3.56 (2H, d, J=6.4 Hz), 3.57 (2H, s), 6.25 (1H, brs), 6.85 (1H, d, J=10.1 Hz), 6.93-7.02 (3H, m), 7.31-7.38 (4H, m), 7.45 (1H, dd, J=8.6, 15.1 Hz)

### Preparation 39

6-(3-(2,4-Difluorophenyl)-5-[(2-hydroxy-1,1-dimethylethyl)amino]-1H-pyrazol-4-yl]-2-(2-methylphenyl)pyridazin-3(2H)-one was obtained according to a similar manner to Example 1 mentioned below.

Mass ESI (+) 452 (M+1)

¹H-NMR (CDCl₃) δ 1.11 (6H, si, 2.21 (3H, s), 3.55 (2H, m), 6.35 (1H, brs), 6.84 (1H, d, J=9.5 Hz), 6.92-6.98 (3H, m), 7.31-7.37 (4H, m), 7.42 (1H, dd, J=7.8, 14.3 Hz)

### Preparation 40

6-[3-(2,4-Difluorophenyl)-5-{[(2S)-2-hydroxypropyl]amino}-1H-pyrazol-4-yl]-2-(2-methylphenyl)pyridazin-3(2H)-one was obtained according to a similar manner to Example 1 mentioned below.

¹H-NMR (CDCl₃) δ 1.11 (6H, s), 2.21 (3H, s), 3.55 (2H, m), 16.35 (1H, brs), 6.84 (1H, d, J=9.5 Hz), 6.92-6.98 (3H, m), 7.31-7.37 (4H, m), 7.42 (1H, dd, J=7.8, 14.3 Hz)

### Preparation 41

6-[3-(2,4-Difluorophenyl)-5-{[(2R)-2-hydroxypropyl]amino}-1H-pyrazol-4-yl]-2-(2-methylphenyl)pyridazin-3(2H)-one was obtained according to a similar manner to Example 1 mentioned below.

¹H-NMR (CDCl₃) δ 1.09 (3H, d, J=6.0 Hz), 2.20 (3H, s), 3.10-3.17 (1H, m), 3.22-3.27 (1H, m), 3.88-3.94 (1H, m), 6.16 (1H, brs), 6.85 (1H, d, J=9.6 Hz), 6.92-6.99 (3H, m), 7.32-7.37 (4H, m), 7.43 (1H, dd, J=8.3, 15.6 Hz)

### Preparation 42

6-{3-(2,4-Difluorophenyl)-5-[(2S)-2-(hydroxymethyl)pyrrolidin-1-yl]-1H-pyrazol-4-yl}-2-(2-methylphenyl)pyridazin-3(2H)-one was obtained according to a similar manner to Example 1 mentioned below.

Mass ESI (+) 464 (M+1)

¹H-NMR (CDCl₃) δ 1.68-1.74 (1H, m), 1.81-1.91 (2H, m), 2.02-2.10 (1H, m), 2.07 (3H, s), 3.08 (1H, dd, J=6.9, 16.5 Hz), 3.40 (1H, dd, J=6.9, 15.1 Hz), 3.64 (1H, dd, J=6.4, 11.0 Hz), 3.75 (1H, dd, J=3.2, 11.0 Hz), 3.94-3.99 (1H, m), 6.82-6.91 (2H, m), 6.96 (1H, d, J=9.6 Hz), 7.07-7.13 (1H, m), 7.27-7.35 (4H, m), 7.39 (1H, dd, J=8.4, 14.7 Hz)

### Preparation 43

6-[3-(4-Fluorophenyl)-5-{[(1R)-2-hydroxy-1-methylethyl]amino}-1H-pyrazol-4-yl]-2-(2-methylphenyl)pyridazin-3(2H)-one was obtained according to a similar manner to Example 1 mentioned below.

Mass ESI (+) 420 (M+1)

¹H-NMR (CDCl₃) δ 1.04 (3H, d, J=6.0 Hz), 2.23 (3H, s), 3.36-3.45 (1H, m), 3.65 (2H, d, J=8.2 Hz), 6.01 (1H, brs), 6.83 (1H, d, J=9.6-Hz), 6.98 (1H, d, J=9.6 Hz), 7.14 (2H, dd, J=8.7 Hz, J=8.7 Hz), 7.28-7.42 (4H, m), 7.45 (2H, dd, J=5.5 Hz, J=8.7 Hz).

### Preparation 44

6-[3-(4-Fluorophenyl)-5-({[4-(hydroxymethyl)tetrahydro-2H-thiopyran-4-yl]methyl}amino)-1H-pyrazol-4-yl]-2-(2-methylphenyl)pyridazin-3(2H)-one was obtained according to a similar manner to Example 1 mentioned below.

Mass ESI (+) 506 (M+1)

¹H-NMR (CDCl₃) δ 1.42 (4H, br), 2.10 (3H, s), 2.41-2.44 (4H, m), 3.06 (2H, d, J=5 Hz), 3.12 (2H, d, J=6 Hz), 4.16 (1H, t, J=6 Hz), 5.57 (1H, br), 6.92 (1H, m), 7.03 (1H, m), 7.34-7.40 (6H, m), 7.53 (2H, m), 12.29 (1H, s)

### Preparation 45

6-[5-({[1-(Bromomethyl)cyclohexyl]methyl}amino)-3-(4-fluorophenyl)-1H-pyrazol-4-yl)-2-(2-methylphenyl)pyridazin-3(2H)-one was obtained according to a similar manner to Example 1 mentioned below.

Mass ESI (+) 550 (M+1)

¹H-NMR (DMSO-d₆) δ 1.12-1.38 (10H, m), 2.09 (3H, s), 3.11-3.19 (2H, m), 6.92 (1H, d, J=10.0 Hz), 7.01 (1H, d, J=10.0 Hz), 7.32-7.41 (6H, m), 7.52-7.58 (2H, m)

### Preparation 46

6-[3-(4-Fluorophenyl)-5-({[4-(hydroxymethyl)tetrahydro-2H-pyran-4-yl]methyl}amino)-1H-pyrazol-4-yl]-2-(2-methylphenyl)pyridazin-3(2H)-one was obtained according to a similar manner to Example 1 mentioned below.

Mass ESI (+) 490 (M+1)

¹H-NMR (CDCl₃) δ 1.48-1.56 (2H, m), 1.68-1.79 (2H, m), 2.01 (2H, t, J=6 Hz), 2.25 (3H, s), 3.07-3.22 (2H, m), 3.60-3.75 (2H, m), 4.18 (2H, t, J=6 Hz), 6.79-6.83 (1H, m), 6.83 (1H, d, J=10 Hz), 7.06 (1H, d, J=10 Hz), 7.14-7.19 (2H, m), 7.35-7.39 (4H, m), 7.48-7.53 (2H, m)

### Preparation 47

6-{5-[({4-[(Benzyloxy)methyl]-1,1-dioxidotetrahydro-2H-thiopyran-4-yl}methyl)amino]-3-(4-fluorophenyl)-1H-pyrazol-4-yl}-2-(2-methylphenyl)pyridazin-3(2H)-one was obtained according to a similar manner to Example 1 mentioned below.

Mass ESI (+) 628 (M+1)

¹H-NMR (CDCl₃) δ 1.83-1.96 (4H, m), 2.18 (3H, s), 2.78-2.84 (2H, m), 3.04-3.08 (2H, m), 3.08 (2H, s), 3.50 (2H, d, J=6 Hz), 4.11 (2H, s), 6.35 (1H, br), 6.83 (1H, d, J=10 Hz), 6.92 (1H, d, J=10 Hz), 7.13-7.17 (4H, m), 7.27-7.43 (9H, m)

### Preparation 48

6-[5-{[(1S)-1-({[tert-Butyl(diphenyl)silyl]oxy}methyl)-3-hydroxypropyl]amino}-3-(4-fluorophenyl)-1H-pyrazol-4-yl]-2-(2-methylphenyl)pyridazin-3(2H)-one was obtained according to a similar manner to Example 1 mentioned below.

¹H-NMR (DMSO-d₆) δ 0.80 (9H, s), 1.52-1.62 (1H, m), 1.70-1.83 (1H, m), 2.07 (3H, s), 3.46-3.86 (5H, m), 4.46 (1H, brs), 5.53 (1H, brs), 6.92 (1H, d, J=10 Hz), 7.03 (1H, d, J=10 Hz), 7.24-7.56 (18H, m), 12.27 (1H, brs)

### Preparation 49

6-[5-{[(1R)-1-({[tert-Butyl(diphenyl)silyl]oxy}methyl)-3-hydroxypropyl]amino}-3-(4-fluorophenyl)-1H-pyrazol-4-yl]-2-(2-methylphenyl)pyridazin-3(2H)-one was obtained according to a similar manner to Example 1 mentioned below.

¹H-NMR (DMSO-d₆) δ 0.80 (9H, s), 1.52-1.62 (1H, m), 1.70-1.83 (1H, m), 2.07 (3H, s), 3.46-3.86 (5H, m), 4.46 (1H, brs), 5.53 (1H, brs), 6.92 (1H, d, J=10 Hz), 7.03 (1H, d, J=10 Hz), 7.24-7.56 (18H, m), 12.27 (1H, brs)

### Preparation 50

tert-Butyl 4-[{3-(4-fluorophenyl)-4-[1-(2-methylphenyl)-6-oxo-1,6-dihydropyridazin-3-yl]-1H-pyrazol-5-yl}amino)methyl)-4-(hydroxymethyl)piperidine-1-carboxylate was obtained according to a similar manner to Example 1 mentioned below.

Mass ESI (+) 611 (M+Na)

### Preparation 51

6-[3-(4-Fluorophenyl)-5-{[4-(2-hydroxyethyl)tetrahydro-2H-pyran-4-yl]amino}-1H-pyrazol-4-yl)-2-(2-methylphenyl)pyridazin-3(2H)-one was obtained according to a similar manner to Example 1 mentioned below.

Mass ESI (+) 472 (M+1)

¹H-NMR (CDCl₃) δ 1.52-1.64 (2H, m), 1.80-1.90 (2H, m) 1.83-1.87 (2H, m), 2.24 (3H, s), 3.09-3.28 (2H, m), 3.38-3.50 (2H, m), 3.71 (2H, d, J=6 Hz), 6.85 (1H, d, J=10 Hz), 6.97 (1H, d, J=10 Hz), 7.18-7.25 (2H, m), 7.32-7.41 (4H, m), 7.44-7.48 (2H, m)

### Preparation 52

6-(3-(4-Fluorophenyl)-5-[(3-hydroxy-2,2-dimethylpropyl)amino]-1H-pyrazol-4-yl}-2-(2-methylphenyl)pyridazin-3(2H)-one was obtained according to a similar manner to Example 1 mentioned below.

Mass ESI (+) 448 (M+1)

¹H-NMR (CDCl₃) δ 0.62 (6H, s), 2.10 (3H, s), 2.98-3.00 (4H, m), 4.59 (1H, t, J=6 Hz), 6.91-7.04 (2H, m), 7.34-7.39 (6H, m), 7.53 (2H, m)

### Preparation 53

6-{3-(4-Fluorophenyl)-5-[(2-hydroxyethyl)amino]-1H-pyrazol-4-yl}-2-(2-methylphenyl)pyridazin-3(2H)-one was obtained according to a similar manner to Example 1 mentioned below.

¹H-NMR (CDCl₃) δ 2.22 (3H, s), 3.45-3.52 (2H, m), 3.72-3.78 (2H, m), 6.82 (1H, d, J=9.5 Hz), 6.89 (1H, d, J=10.0 Hz), 7.06-7.17 (2H, m), 7.31-7.42 (8H, m)

### Preparation 54

6-{[1S-{[(1S)-2-(Benzyloxy)-1-(hydroxymethyl)ethyl]amino}-3-(4-fluorophenyl)-1H-pyrazol-4-yl]-2-(2-methylphenyl)pyridazin-3(2H)-one was obtained according to a similar manner to Example 1 mentioned below.

Mass ESI (+) 548 (M+Na)

¹H-NMR (CDCl₃) δ 2.18 (3H, s), 3.27-4.41 (7H, m), 6.52-7.63 (15H, m)

### Preparation 55

6-[5-{[(1R)-2-(Benzyloxy)-1-(hydroxymethyl)ethyl]amino}-3-(4-fluorophenyl)-1H-pyrazol-4-yl]-2-(2-methylphenyl)pyridazin-3(2H)-one was obtained according to a similar manner to Example 1 mentioned below.

Mass ESI (+) 548 (M+Na)

¹H-NMR (CDCl₃) δ 2.20 (3H, s), 3.50 (2H, m), 3.70 (2H, m), 3.93 (1H, brs), 4.22 (2H, s), 6.85 (1H, d), 7.08 (3H, m), 7.19-7.43 (11H, m)

### Preparation 56

(2S)-2-({3-(4-Fluorophenyl)-4-[L-(2-methylphenyl)-6-oxo-1,6-dihydropyridazin-3-yl]-1H-pyrazol-5-yl}amino)-3-methylbutyl acetate was obtained according to a similar manner to Example 1 mentioned below.

Mass ESI (+) 490 (M+1)

¹H-NMR (CDCl₃) δ 0.54-0.66 (3H, m), 0.80-0.90 (3H, m), 1.78-1.88 (1H, m), 1.98 (3H, s), 2.22 (3H, s), 3.46-3.64 (1H, m), 3.93-4.02 (1H, m), 4.18-3.25 (1H, m), 6.13-6.31 (1H, m), 6.82 (1H, d, J=9.5 Hz), 7.02 (1H, d, J=9.5 Hz), 7.20 (2H, dd, J=8.5 Hz, J=8.5 Hz), 7.30-7.39 (4H, m), 7.49 (2H, dd, J=5.5 Hz, J=8.5 Hz)

### Preparation 57

6-{5-[(3-Bromo-2,2-difluoropropyl)amino]-3-(4-fluorophenyl)-1H-pyrazol-4-yl}-2-(2-methylphenyl)pyridazin-3(2H)-one was obtained according to a similar manner to Example 1 mentioned below.

¹H-NMR (CDCl₃) δ 2.23 (3H, s), 3.55 (2H, t, J=13.5 Hz), 3.89 (2H, J=13.0 Hz, J=6.5 Hz), 5.98 (1H, m), 6.85 (1H, d, J=9.5 Hz), 6.98 (1H, d, J=9.5 Hz), 7.24 (2H, dd, J=9.0 Hz, J=9.0 Hz), 7.31-7.42 (4H, m), 7.46 (2H, dd, J=5.5 Hz, J=9.0 Hz), 9.09 (1H, brs)

### Preparation 58

6-[5-({[2-(Bromomethyl)-1,3-dioxolan-2-yl]methyl}amino)-3-(4-fluorophenyl)-1H-pyrazol-4-yl]-2-(2 methylphenyl)pyridazin-3(2H)-one was obtained according to a similar manner to Example 1 mentioned below.

¹H-NMR (CDCl₃) δ 2.23 (3H, s), 3.39 (2H, s), 3.54-3.66 (2H, m), 3.69-3.89 (2H, m), 3.91-4.05 (2H, m), 6.07 (1H, brs), 6.83 (1H, d, J=10.0 Hz), 6.99 (1H, d, J=10.0 Hz), 7.20 (2H, dd, J=9.0 Hz, J=9.0 Hz), 7.33-7.43 (4H, m), 7.47 (2H, dd, J=5.5 Hz, J=9.0 Hz)

### Preparation 59

6-{3-(2,4-Difluorophenyl)-5-[(3-hydroxy-1-methylpropyl)amino]-1H-pyrazol-4-yl}-2-(2-methylphenyl)pyridazin-3(2H)-one was obtained according to a similar manner to Example 10 mentioned below.

¹H-NMR (CDCl₃) δ 1.09 (3H, d, J=5.5 Hz), 1.71-1.77 (2H, m), 2.23 (3H, s), 3.51-3.55 (1H, m), 3.59-3.66 (2H, m), 5.99 (1H, brs), 6.84 (1H, d, J=10.1 Hz), 6.90-6.98 (3H, m), 7.27-7.43 (5H, m)

### Preparation 60

6-[3-(4-Fluorophenyl)-5-({[4-(hydroxymethyl)-1,1-dioxidotetrahydro-2H-thiopyran-4-ylmethyl}amino)-1H-pyrazol-4-yl]-2-(2-methylphenyl)pyridazin-3(2H)-one was obtained according to a similar manner to Example 95 mentioned below.

Mass ESI (+) 538 (M+1)

¹H-NMR (DMSO-d₆) δ 1.70 (4H, m), 2.10 (3H, s), 2.90-3.02 (4H, m), 3.16 (2H, s), 3.23 (2H, d, J=6 Hz), 6.94 (1H, d, J=10 Hz), 7.06 (1H, d, J=10 Hz), 7.30-7.40 (6H, m), 7.51-7.55 (2H, m)

### Preparation 61

Benzyl 4-({3-(4-fluorophenyl)-4-[1-(2-methylphenyl)-6-oxo-1,6-dihydropyridazin-3-yl]-1H-pyrazol-5-yl}amino)-4-(2-hydroxyethyl)piperidine-1-carboxylate was obtained according to a similar manner to Example 123 mentioned below.

¹H-NMR (DMSO-d₆) δ 1.36 (2H, t), 1.91 (3H, brs), 2.07 (3H, s), 2.67 (2H, brs), 3.51 (2H, m), 4.21 (1H, t), 5.03 (2H, brs), 5.82 (1H, s), 6.55 (1H, s), 6.94 (1H, d), 7.03 (1H, d), 7.25-7.46 (9H, m), 7.57 (2H, m), 7.69 (4H, m)

### EXAMPLE 1

A mixture of 6-[1-bromo-2-(2,4-difluorophenyl)-2-oxoethyl]-2-(2-methylphenyl)-3(2H)-pyridazinone (210 mg), 3-[(dimethylamino)methyl]-1-azetidinecarbothiohydrazide (113 mg) in glacial acetic acid (1.5 mL) was heated at 55° C. to 60° C. for 1.5 h. The mixture was poured into water (20 mL), neutralized with sodium hydrogencarbonate and extracted with ethyl acetate (30 mL). The extract was concentrated under reduced pressure. The residue was purified by flash column chromatography on SiO₂ (eluent; 0% to 4% methanol in chloroform) to give 6-{2-(2,4-difluorophenyl)-6-[(dimethylamino)methyl]-4,5,6,7-tetrahydropyrazolo[1,5-a]pyrimidin-3-yl}-2-(2-methylphenyl)-3(2H)-pyridazinone (167 mg) as yellow amorphous solid.

Mass ESI (+) 477 (M+1)

¹H-NMR (500 MHz, CDCl₃)δ 2.21 (3H, s), 2.24 (6H, s), 2.26-2.43 (3H, m), 3.09 (1H, t, J=8.9 Hz), 3.48 (1H, d, J=12.3 Hz), 3.82 (1H, dd, J=8.1 Hz, 12.4 Hz), 4.27 (1H, dd, J=5.6 Hz, 12.4 Hz), 5.90 (1H, brs), 6.83 (1H, d, J=9.6 Hz), 6.92-6.97 (2H, m), 7.00-7.04 (1H, m), 7.34-7.38 (4H, m), 7.55 (1H, dd, J=8.1 Hz, 14.7 Hz)

### EXAMPLE 2

A mixture of 6-{2-(2,4-difluorophenyl)-6-[(dimethylamino)methyl]-4,5,6,7-tetrahydropyrazolo[1,5-a]pyrimidin-3-yl}-2-(2-methylphenyl)-3(2H)-pyridazinone (48 mg) and 2,3-dichloro-5,6-dicyano-p-benzoquinone (23 mg) in dioxane (1 mL) was stirred at room temperature overnight. To the mixture was added water (10 mL), and the mixture was extracted with ethyl acetate (15 mL). The extract was concentrated under reduced pressure. The residue was purified by flash column chromatography on SiO₂ (eluent; ethyl acetate to 4% MeOH in chloroform) to give 6-{2-(2,4-difluorophenyl)-6-[(dimethylamino)methyl]pyrazolo[11,5-a]pyrimidin-3-yl}-2-(2-methylphenyl)-3(2H)-pyridazinone as yellow oil (22 mg).

Mass ESI (+) 473 (M+1)

¹H-NMR (500 MHz, CDCl₃) δ 2.05 (3H, s), 2.33 (6H, s), 3.55 (2H, s), 6.67 (1H, t, J=10.2 Hz), 6.87 (1H, t, J=8.2 Hz), 7.01 (1H, d, J=7.8 Hz), 7.14-7.19 (1H, d, J=9.6 Hz), 7.36 (3H, m), 7.52 (1H, dd, J=7.5 Hz, 15.2 Hz), 8.31 (2H, d, J=9.5 Hz), 8.64 (2H, d, J=6.9 Hz)

### EXAMPLE 3

6-[2-(4-Fluorophenyl)-6-(hydroxymethyl)-4,5,6,7-tetrahydropyrazolo[1,5-a]pyrimidin-3-yl]-2-(2-methylphenyl)-3(2H)-pyridazinone was obtained according to a similar manner to Example 1.

Mass ESI (+) 432 (M+1)

¹H-NMR (500 MHz, CDCl₃)-1.69 (1H, brs), 2.23 (3H, s), 2.40-2.50 (1H, m), 3.20-3.30 (1H, m), 3.46-5.30 (1H, m), 3.72-3.78 (2H, m), 3.98 (1H, dd, J=7.3, 13.0 Hz), 4.25 (111, dd, J=5.2, 13.0 Hz), 5.81 (1H, brs), 6.81 (1H, d, J=9.5 Hz), 7.02 (1H, d, J=9.5 Hz), 7.15 (2H, dd, J=8.2, 8.5 Hz), 7.31-7.41 (4H, m), 7.50 (2H, dd, J=5.6, 8.2 Hz)

### EXAMPLE 4

To a suspension of 6-[2-(4-fluorophenyl)-6-(hydroxymethyl)-4,5,6,7-tetrahydropyrazolo[1,5-a]pyrimidin-3-yl]-2-(2-methylphenyl)-3(2H)-pyridazinone (0.216 mg) in tetrahydrofuran (2 mL) were added imidazole (85 mg) and triphenylphosphine (197 mg), and the suspension was stirred at room temperature for 5 minutes. To the suspension was added dropwise a solution of iodine (190 mg) in tetrahydrofuran (1 mL), and the mixture was stirred at room temperature for 1.5 h. To the reaction mixture were added EtOAc (50 mL), and the solution was washed successively with 3% aqueous Na₂S₂O₃ (20 mL), saturated aqueous NaHCO₃ solution (20 mL) and brine (20 mL). The organic layer was dried over anhydrous MgSO₄ and the insoluble substance was filtered off. The filtrate was concentrated in vacuo. The residue was purified by flash column chromatography (gradient elution: hexane/EtOAc (w/w) 0% to 100%) to give 6-[2-(4-fluorophenyl)-6-iodomethyl-4,5,6,7-tetrahydropyrazolo[1,5-a]pyrimidin-3-yl]-2-(2-methylphenyl)-3(2H)-pyridazinone (0.211 g) as yellow solid.

¹H-NMR (500 MHz, CDCl₃) δ 2.23 (3H, s), 2.44-2.52 (1H, m), 3.22-3.26 (1H, m), 3.26 (2H, d, J=6.9 Hz), 3.51-3.57 (1H, m), 3.96 (1H, dd, J=7.6, 13.0 Hz), 4.34 (1H, dd, J=5.1, 13.0 Hz), 5.84 (1H, brs), 6.81 (1H, d, J=9.2 Hz), 7.02 (1H, d, J=9.2 Hz), 7.16 (2H, dd, J=8.2, 8.5 Hz), 7.32-7.42 (4H, m), 7.50 (2H, dd, J=5.0, 8.2 Hz)

### EXAMPLE 5

To a suspension of 6-[2-(4-fluorophenyl)-6-iodomethyl-(4,5,6,7-tetrahydropyrazolo[1,5-a]pyrimidin-3-yl}]-2-(2-methylphenyl)]-3(2H)-pyridazinone (50 mg) in CH₃CN (1 mL) were added 4-dimethylaminopiperidine (36 mg) and K₂CO₃ (25.6 mg), and the suspension was stirred at 80° C. for 2.5 h. To the suspension was added 4-dimethylaminopiperidine (36 mg), and the suspension was additionally stirred for 8 h. To the reaction mixture were added EtOAc (30 mL), and the solution was extracted with 10% citric acid (20 mL×2). The extracts were combined, and the solution was basified with NaHCO₃. The suspension was extracted with EtOAc (20 mL×3), and the organic layers were combined. The solution was dried over anhydrous MgSO₄ and filtered off. The filtrate was concentrated in vacuo. To the residue was added 4 M HCl in EtOAc (2 mL) to give 6-[6-{[4-(dimethylamino)-1-piperidinyl]methyl}-2-(4-fluorophenyl)-4,5,6,7-tetrahydropyrazolo[1,5-a]pyrimidin-3-yl]-2-(2-methylphenyl)-3(2H)-pyridazinone dihydrochloride (25 mg).

Mass ESI (+) 542 (M+1)

¹H-NMR (500 MHz, CDCl₃) δ 2.09 (3H, s), 2.16-2.32 (4H, m), 2.68-2.81 (7H, m), 2.90-3.02 (2H, m), 3.10-3.22 (3H, m), 3.47-3.54 (1H, m), 3.62-4.07 (4H, m), 4.30-4.41 (1H, m), 6.10 (1H, brs), 6.95 (1H, d, J=9.5 Hz), 7.10 (1H, d, J=9.5 Hz), 7.24 (2H, dd, J=8.6, 8.9 Hz), 7.31-7.40 (4H, m), 7.49 (2H, dd, J=5.5, 8.5 Hz), 10.8 (1H, brs), 11.1 (1H, brs)

### EXAMPLE 6

A mixture of 6-[5-(ethylamino)-3-(4-fluorophenyl)-1-(2-hydroxyethyl)-1H-pyrazol-4-yl]-2-(2-methylphenyl)-3(2H)-pyridazinone (858 mg), imidazole (337 mg, 4.95 mmol) and triphenylphosphine (779 mg) in tetrahydrofuran (5.0 ml) was stirred at room temperature for 3 h. To the mixture was added dropwise a solution of iodine (754 mg) in THF (5.0 mL). The mixture was stirred overnight. To the mixture were added THF (4.0 mL), imidazole (236 mg) and triphenylphosphine (545 mg). The mixture was stirred at room temperature for 20 minutes. To the mixture was added dropwise a solution of iodine (754 mg) in THF (4.0 ml) at room temperature, and the mixture was stirred for 6 h. The resulting precipitate was removed by filtration. To the filtrate was added EtOAc (50 ml). The mixture was washed successively with 5% aqueous Na₂S₂O₃ solution (30 ml), 5% aqueous NaHCO₃ solution (30 ml) and brine (30 mL). The organic layer was concentrated under reduced pressure. The residue was purified by flash column chromatography (gradient elution: EtOAc/hexane=50% to 85%). To the crystalline residue was added isopropyl ether, and the solid was filtered off. To the solid was added 10% HCl (100 mL) and EtOAc (50 mL). The aqueous layer was separated, washed with Et₂O and neutralized with NaOH. The mixture was extracted with EtOAc (100 mL). The extract was dried over anhydrous MgSO₄ and concentrated under reduced pressure. To the crystalline residue was added hexane, and the solid was filtered off to give 6-[1-ethyl-6-(4-fluorophenyl)-2,3-dihydro-1H-imidazo [1,2-b]pyrazol-7-yl]-2-(2-methylphenyl)-3(2H)-pyridazinone as pale yellow prisms (460 mg).

Mass ESI (+) 416 (M+1)

¹H-NMR (500 MHz, CDCl₃) δ 0.97 (3H, t, J=6.8 Hz), 2.20 (3H, s), 3.27 (2H, q, J=6.9 Hz), 3.81 (2H, t, J=8.6 Hz), 4.20 (2H, t, J=8.2 Hz), 6.86 (1H, d, J=9.7 Hz), 6.97 (1H, d, J=9.7 Hz), 7.08 (2H, dd, J=8.7, 8.7 Hz), 7.26-7.36 (4H, m), 7.46 (2H, dd, J=5.5, 8.7 Hz)

### EXAMPLE 7

A mixture of tert-butyl 3-[{3-(4-fluorophenyl)-4-[1-(2-methylphenyl)-6-oxo-1,6-dihydro-3-pyridazinyl]-1H-pyrazol-5-yl}(3-hydroxypropyl)amino]propanoate (175 mg), triphenylphosphine (126 mg) and diethyl azodicarboxylate (75 μL) in THF (6 ml) was stirred at room temperature for 24 h. The mixture was concentrated under reduced pressure. The residue was purified by flash column chromatography (gradient elution: EtOAc/hexane=20% to 95%) to give tert-butyl 3-[2-(4-fluorophenyl)-3-[1-(2-methylphenyl)-6-oxo-1,6-dihydro-3-pyridazinyl]-6,7-dihydropyrazolo[1,5-a]pyrimidin-4(5H)-yl]propanoate (153 mg) as yellow amorphous.

¹H-NMR (500 MHz, CDCl₃) δ 1.39 (9H, s), 2.16 (3H, s), 2.15-2.23 (2H, m), 2.28 (2H, t, J=6.9 Hz), 3.28 (2H, t, J=5.6 Hz), 3.49-3.54 (2H, m), 4.14 (2H, t, J=6.5 Hz), 6.92 (1H, d, J=9.6 Hz), 7.01 (2H, dd, J=8.7, 8.7 Hz), 7.12 (1H, d, J=9.6 Hz), 7.25-7.35 (4H, m), 7.39 (2H, dd, J=5.5, 8.8 Hz)

### EXAMPLE 8

To a solution of tert-butyl 3-[2-(4-fluorophenyl)-3-(1-(2-methylphenyl)-6-oxo-1,6-dihydro-3-pyridazinyl)-6,7-dihydropyrazolo[1,5-a]pyrimidin-4(5H)-yl]propanoate (145 mg) in chloroform (6 ml) was added trifluoroacetic acid (0.5 mL), and the mixture was stirred at room temperature for 2 h. To the mixture was added water (10 mL), and the mixture was neutralized with NaHCO₃. The organic layer was separated and concentrated under reduced pressure to give 3-[2-(4-fluorophenyl)-3-[1-(2-methylphenyl)-6-oxo-1,6-dihydro-3-pyridazinyl]-6,7-dihydropyrazolo[1,5-a]pyrimidin-4(5H)-yl]propanoic acid (110 mg) as yellow amorphous.

¹H-NMR (500 MHz, CDCl₃) δ 2.13 (3H, s), 2.16-2.19 (2H, m), 2.36 (2H, t, J=7.5 Hz), 3.26 (2H, t, J=5.5 Hz), 3.56 (2H, brs), 4.14 (2H, t, J=5.8 Hz), 6.96 (1H, d, J=9.6 Hz), 7.01 (2H, dd, J=8.7, 8.7 Hz), 7.11 (1H, d, J=9.6 Hz), 7.26-7.33 (4H, m), 7.37 (2H, dd, J=5.5, 8.7 Hz)

### EXAMPLE 9

A mixture of ethyl 3-{6-(4-fluorophenyl)-7-[1-(2-methylphenyl)-6-oxo-1,6-dihydro-3-pyridazinyl]-2,3-dihydro-1H-imidazo[1,2-b]pyrazol-1-yl}propanoate (450 mg) and 10% aqueous NaOH solution (4 mL) in ethanol (10 mL) was stirred at 60° C. for 50 minutes. The solvent was removed under reduced pressure. To the residue was added water (10 mL) The mixture was neutralized with citric acid and extracted with EtOAc (30 mL). The extract was dried over anhydrous MgSO₄ and concentrated under reduced pressure to give 3-{6-(4-fluorophenyl)-7-[1-(2-methylphenyl)-6-oxo-1,6-dihydro-3-pyridazinyl]-2,3-dihydro-1H-imidazo[1,2-b]pyrazol-1-yl}propanoic acid (380 mg) as pale yellow powder.

¹H-NMR (500 MHz, CDCl₃) δ 2.18 (3H, s), 2.38 (2H, t, J=6.9 Hz), 3.55 (2H, brs), 3.85 (2H, t, J=8.3 Hz), 4.20 (2H, t, J=7.9 Hz), 6.91 (1H, d, J=9.7 Hz), 6.98 (1H, d, J=9.7 Hz), 7.07 (2H, dd, J=8.6, 8.6 Hz), 7.27-7.33 (4H, m), 7.42 (2H, dd, J=5.0, 8.1 Hz)

### EXAMPLE 10

To a solution of 3-{6-(4-fluorophenyl)-7-[1-(2-methylphenyl)-6-oxo-1,6-dihydro-3-pyridazinyl]-2,3-dihydro-1H-imidazo[1,2-b]pyrazol-1-yl}propanoic acid (0.87 g) in THF (15 mL) was added NaBH₄ (0.22 g), following BF₃-Et₂O complex (0.72 mL) at room temperature. The mixture was stirred at room temperature for 5.5 h. To the mixture were added dichloromethane (50 mL) and aqueous saturated NaHCO₃ solution (30 mL). The mixture was stirred at room temperature overnight. The organic layer was separated and concentrated under reduced pressure. The residue was purified by flash column chromatography (gradient elution: methanol/chloroform=5% to 10%) to give 6-[6-(4-fluorophenyl)-1-(3-hydroxypropyl)-2,3-dihydro-1H-imidazo[1,2-b]pyrazol-7-yl]-2-(2-methylphenyl)-3(2H)-pyridazinone as yellow amorphous (0.22 g).

Mass ESI (+) 446 (M+1)

¹H-NMR (500 MHz, CDCl₃) δ 1.34 (1H, brs), 1.61-1.67 (2H, m), 2.21 (3H, s), 3.34-3.40 (4H, m), 3.83 (2H, t, J=8.7 Hz), 4.21 (2H, t, J=8.2 Hz), 6.85 (1H, d, J=9.8 Hz), 6.95 (1H, d, J=9.8 Hz), 7.08: (2H, dd, J=8.2, 8.2 Hz), 7.33-7.38 (4H, m), 7.44 (2H, dd, J=5.5, 8.4 Hz)

### EXAMPLE 11

To a suspension of 3-{6-(4-fluorophenyl)-7-[1-(2-methylphenyl)-6-oxo-1,6-dihydro-3-pyridazinyl)-2,3-dihydro-1H-imidazo[1,2-b]pyrazol-1-yl}propanoic acid (96 mg) in dichloromethane (5 mL) was added catalytic amount of DMF. To the mixture was added dropwise oxalyl chloride (23 μL) at room temperature. The mixture was stirred at room temperature for 1.5 h. The solvent was removed under reduced pressure. The residue was dissolved in chloroform (5 mL). To a solution of morpholine (55 μL) in chloroform (5 mL) was added slowly the solution of acid chloride in chloroform. The mixture was stirred for 40 minutes. To the mixture was added water (5 mL). The organic layer was separated and concentrated under reduced pressure. The residue was purified by flash column chromatography (methanol/chloroform=8%) to give 6-{6-(4-fluorophenyl)-1-[3-(4-morpholinyl)-3-oxopropyl]-2,3-dihydro-1H-imidazo[1,2-b]pyrazol-7-yl}-2-(2-methylphenyl)-3(2H)-pyridazinone (35 mg) as yellow amorphous.

Mass ESI (+) 529 (M+1)

¹H-NMR (500 MHz, CDCl₃) δ 2.21-2.27 (5H, m), 2.92-2.96 (2H, m), 3.48-3.52 (6H, m), 3.62 (2H, t, J=4.5 Hz), 3.96 (2H, t, J=7.8 Hz), 4.16 (2H, t, J=8.1 Hz), 6.84 (1H, d, J=9.6 Hz), 6.93 (1H, d, J=9.5 Hz), 7.09 (2H, dd, J=8.1, 8.1 Hz), 7.30-7.38 (4H, m), 7.43 (2H, dd, J=5.5, 8.4 Hz)

### EXAMPLE 12

To a mixture of 6-[6-(4-fluorophenyl)-1-(3-hydroxypropyl)-2,3-dihydro-1H-imidazo[1,2-b]pyrazol-7-yl]-2-(2-methylphenyl)-3(2H)-pyridazinone (178 mg) and triethylamine (78 μL) in dichloromethane (10 mL) was added methanesulfonyl chloride (37 μL). The mixture was stirred for 1.5 h. To the mixture was added dichloromethane (10 mL), and the mixture was washed with successive water (10 mL) and 5% aqueous NaHCO₃ solution (10 mL). The mixture was dried over anhydrous MgSO₄ and concentrated under reduced pressure. The residue was purified by flash column chromatography (gradient elution: methanol/chloroform=0% to 10%) to give 3-[6-(4-fluorophenyl)-7-(1-(2-methylphenyl)-6-oxo-1,6-dihydropyridazin-3-yl)-2,3-dihydro-1H-imidazo[1,2-b]pyrazol-1-yl]propyl methanesulfonate (107 mg) as pale yellow amorphous.

¹H-NMR (CDCl₃) δ 1.80-1.86 (2H, m), 2.21 (3H, s), 2.89 (3H, s), 3.37 (2H, t, J=6.7 Hz), 3.82 (2H, t, J=6.3 Hz), 3.85 (2H, t, J=8.4 Hz), 4.23 (2H, t, J=8.2 Hz), 6.85 (1H, d, J=9.6 Hz), 6.93 (1H, d, J=9.8 Hz), 7.10 (2H, dd, J=8.6, 8.6 Hz), 7.30-7.39 (4H, m), 7.44 (2H, dd, J=5.4, 8.7 Hz)

### EXAMPLE 13

A mixture of 3-[6-(4-fluorophenyl)-7-(1-(2-methylphenyl)-6-oxo-1,6-dihydropyridazin-3-yl)-2,3-dihydro-1H-imidazo[1,2-b]pyrazol-1-yl]propyl methanesulfonate (52 mg), morpholine (10 μL) and anhydrous K₂CO₃ (17 mg) in CH₃CN (4 mL) was refluxed for 2 h. To the mixture were added morpholine (15 μL) and KI (10 mg), and the mixture was refluxed for 2 h. To the mixture was added water (20 mL) The mixture was extracted with EtOAc (30 mL). The extract was concentrated under reduced pressure. The residue was purified by flash column chromatography (gradient elution: methanol/chloroform=5% to 10%) to give 6-{6-(4-fluorophenyl)-1-[3-(4-morpholinyl)propyl]-2,3-dihydro-1H-imidazo[1,2-b]pyrazol-7-yl}-2-(2-methylphenyl)-3(2H)-pyridazinone (52 mg) as pale yellow amorphous.

Mass ESI (+) 515 (M+1)

¹H-NMR (500 MHz, CDCl₃) δ 1.56-1.63 (2H, m), 2.01 (2H, t, J=6.8 Hz), 2.21 (3H, s), 2.25 (4H, brs), 3.27 (2H, brs), 53.66 (4H, brs), 3.81 (2H, t, J=8.3 Hz), 4.20 (2H, t, J=7.9 Hz), 6.84 (1H, d, J=9.6 Hz), 6.94 (1H, d, J=9.6 Hz), 7.08 (2H, dd, J=8.0, 8.0 Hz), 7.31-7.38 (4H, m), 7.44 (2H, dd, J=5.4, 7.7 Hz)

### EXAMPLE 14

### 6-{2-(2,4-Difluorophenyl)-6-[(dimethylamino)methyl]-4,5,6,7-tetrahydropyrazolo[1,5-a]pyrimidin-3-yl}-3(2H)-pyridazinone was obtained according to a similar manner to Example 1

¹H-NMR (500 MHz, CDCl₃) δ 2.28 (s, 6H), 2.30-2.51 (m, 3H), 3.22 (t, 1H, J=11.9 Hz), 3.57 (d, 1H, J=11.5 Hz), 3.86 (dd, 1H, J=7.8 Hz, 12.5 Hz), 4.29 (dd, 1H, J=4.4 Hz, 11.8 Hz), 6.13 (brs, 1H), 6.75 (d, 1H, J=10.1 Hz), 6.89-7.01 (m, 2H), 7.27 (t, 1H, J=14.7 Hz), 7.52 (dd, 1H, J=8.1 Hz, 14.6 Hz)

### EXAMPLE 15

A mixture of 6-[2-(2,4-difluorophenyl)-6-(dimethylaminomethyl)-4,5,6,7-tetrahydropyrazolo[1,5-a]pyrimidin-3-yl]pyridazin-3(2H)-one (73 mg), 2-[(tert-butylamino)sulfonyl]phenylboronic acid (147 mg), cupric acetate monohydrate (8 mg) and pyridine (77 μL) in DMF (1.5 mL) was stirred at room temperature for 5 days. To the mixture was added water (40 mL), and the mixture was extracted with EtOAc (40 mL). The extract was washed with brine (40 mL) and concentrated under reduced pressure. The residue was purified by flash column chromatography (gradient elution: methanol/chloroform=0% to 5%) to give N-(tert-butyl)-2-[3-{2-(2,4-difluorophenyl)-6-[(dimethylamino)methyl]-4,5,6,7-tetrahydropyrazolo[1,5-a]pyrimidin-3-yl}-6-oxo-1(6H)-pyridazinyl)benzenesulfonamide (74 mg) as pale brown amorphous.

Mass ESI (+) 598 (M+1)

¹H-NMR (500 MHz, CDCl₃) δ 1.28 (9H, s), 2.22 (6H, s), 2.28-2.39 (3H, m), 3.04 (1H, brs), 3.44 (1H, d, J=11.0 Hz), 3.77 (1H, brs), 4.24 (1H, d, J=11.0 Hz), 5.36 (1H, s), 6.07 (1H, brs), 6.81 (1H, d, J=10.2 Hz), 6.92 (1H, t, J=10.0 Hz), 6.97-7.01 (2H, m), 7.50 (1H, d, J=7.9 Hz), 7.55 (1H, dd, J=7.8, 14.7 Hz), 7.61 (1H, t, J=7.9 Hz), 7.70 (1H, t, J=7.1 Hz), 8.17 (1H, d, J=7.8 Hz)

### EXAMPLE 16

6-{6-[(Dimethylamino)methyl]-2-(4-fluorophenyl)-4,5,6,7-tetrahydropyrazolo[1,5-a]pyrimidin-3-yl}-2-(2-methylphenyl)-3(2H)-pyridazinone was obtained according to a similar manner to Example 1.

Mass ESI (+) 459 (M+1)

¹H-NMR (500 MHz, CDCl₃) δ 2.22 (3H, s), 2.24 (6H, s), 2.27-2.44 (3H, m), 3.09 (1H, t, J=9.5 Hz), 3.47 (1H, d, J=11.8 Hz), 3.82 (1H, dd, J=7.8 Hz, 12.4 Hz), 4.25 (1H, dd, J=4.4 Hz, 12.2 Hz), 5.83 (1H, brs), 6.8 (1H, d, J=9.4 Hz), 7.02 (1H, d, J=9.9 Hz), 7.15 (2H, t, J=8.6 Hz), 7.33-7.39 (4H, m), 7.51 (2H, dd, J=5.5 Hz, 8.6 Hz)

### EXAMPLE 17

6-{2-(2,5-Difluorophenyl)-6-[(dimethylamino)methyl]-4,5,6,7-tetrahydropyrazolo[1,5-a]pyrimidin-3-yl}-2-(2-methylphenyl)-3(2H)-pyridazinone was obtained according to a similar manner to Example 1.

Mass ESI (+) 477 (M+1)

¹H-NMR (500 MHz, CDCl₃) δ 2.21 (s, 3H), 2.24 (s, 6H), 2.26-2.45 (m, 3H), 3.09 (t, 1H, J=9.7 Hz), 3.47 (d, 1H, J=12.0 Hz), 3.82 (dd, 1H, J=8.1 Hz, 12.8 Hz), 4.27 (dd, 1H, J=5.1 Hz, 12.5 Hz), 5.90 (brs, 1H), 6.38 (d, 1H, J=9.7 Hz), 7.00 (dd, 1H, J=1.4 Hz, 9.6 Hz), 7.13 (t, 2H, J=6.3 Hz), 7.29-7.36 (m, 5H)

### EXAMPLE 18

6-[2-(4-Fluorophenyl)-6-hydroxy-4,5,6,7-tetrahydropyrazolo[1,5-a]pyrimidin-3-yl)-2-(2-methylphenyl)-3(2H)-pyridazinone was obtained according to a similar manner to Example 1.

Mass ESI (+) 418 (M+1)

¹H-NMR (500 MHz, CDCl₃) δ 2.23 (3H, s), 2.42 (1H, brs), 3.35-3.45 (2H, m), 4.17-4.23 (1H, m), 4.26 (1H, dd, J=3.2, 13.3 Hz), 4.41 (1H, brs), 5.85 (1H, brs), 6.81 (1H, d, J=9.9 Hz), 7.02 (1H, d, J=9.9 Hz), 7.12-7.19 (2H, m), 7.31-7.42 (4H, m), 7.48-7.54 (2H, m)

### EXAMPLE 19

6-[6-(Dimethylamino)-2-(4-fluorophenyl)-4,5,6,7-tetrahydropyrazolo[1,5-a]pyrimidin-3-yl]-2-(2-methylphenyl)-3(2H)-pyridazinone was obtained according to a similar manner to Example 1.

Mass ESI (+) 445 (M+1)

¹H-NMR (500 MHz, CDCl₃) δ 2.22 (3H, s), 2.39 (6H, s), 2.86-2.92 (1H, m), 3.27 (1H, t, J=10.3 Hz), 3.50-3.57 (1H, m), 4.06 (1H, dd, J=8.2, 12.4 Hz), 4.32 (1H, dd, J=3.7, 12.6 Hz), 5.78 (1H, brs), 6.79 (1H, d, J=9.6 Hz), 7.01 (1H, d, J=9.6 Hz), 7.12-7.18 (2H, m), 7.30-7.42 (4H, m), 7.46-7.54 (2H, m)

### EXAMPLE 20

6-[2-(4-Fluorophenyl)-6-(4-morpholinyl)-4,5,6,7-tetrahydropyrazolo[1,5-a]pyrimidin-3-yl]-2-(2-methylphenyl)-3(2H)-pyridazinone was obtained according to a similar manner to Example 1.

Mass ESI (+) 445 (M+1)

¹H-NMR (500 MHz, CDCl₃) δ 2.22 (3H, s), 2.58-2.68 (4H, m), 2.96-3.06 (1H, m), 3.27 (1H, t, J=9.6 Hz), 3.52-3.60 (1H, m), 3.72 (4H, t, J=4.6 Hz), 4.04 (1H, dd, J=9.2, 12.1 Hz), 4.34 (1H, dd, J=4.4, 12.1 Hz), 5.78 (1H, brs), 6.80 (1H, d, J=9.9 Hz), 7.02 (1H, d, J=9.9 Hz), 7.12-7.20 (2H, m), 7.32-7.42 (4H, m), 7.47-7.54 (2H, m)

### EXAMPLE 21

6-[2-(4-Fluorophenyl)-6-(isopropylamino)-4,5,6,7-tetrahydropyrazolo[1,5-a]pyrimidin-3-yl]-2-(2-methylphenyl)-3(2H)-pyridazinone was obtained according to a similar manner to Example 1.

Mass ESI (+) 459 (M+1)

¹H-NMR (500 MHz, CDCl₃) δ 1.08 (6H, dd, J=2.7, 6.4 Hz), 2.23 (3H, s), 2.96-3.05 (1H, m), 3.12-3.20 (1H, m), 3.36-3.43 (1H, m), 3.43-3.50 (1H, m), 3.97 (1H, dd, J=5.7, 12.6 Hz), 4.25 (1H, dd, J=4.4, 12.6 Hz), 5.81 (1H, brs), 6.80 (1H, d, J=9.9 Hz), 7.02 (1H, d, J=9.9 Hz), 7.15 (2H, dd, J=8.7, 8.7 Hz), 7.30-7.42 (4H, m), 7.47-7.54 (22H, m)

### EXAMPLE 22

6-{2-(4-Fluorophenyl)-6-[(methylamino)methyl]-4,5,6,7-tetrahydropyrazolo[1,5-a]pyrimidin-3-yl}-2-(2-methylphenyl)-3(2H)-pyridazinone was obtained according to a similar manner to Example 1.

Mass ESI (+) 445 (M+1)

¹H-NMR (500 MHz, CDCl₃) δ 2.22 (3H, s), 2.47-2.49 (4H, m), 2.73 (2H, d, J=7.5 Hz), 3.14-3.19 (1H, m), 3.46-3.50 (1H, m), 3.89 (1H, dd, J=7.7, 12.8 Hz), 4.27 (1H, dd, J=5.0, 12.9 Hz), 5.82 (1H, brs), 6.80 (1H, d, J=9.4 Hz), 7.01 (1H, d, J=9.8 Hz), 7.15 (2H, dd, J=8.8, 8.8 Hz), 7.32-7.38 (4H, m), 7.50 (2H, dd, J=5.4, 8.7 Hz)

### EXAMPLE 23

6-{6-[(Dimethylamino)methyl]-2-(3-methoxyphenyl)-4,5,6,7-tetrahydropyrazolo[1,5-a]pyrimidin-3-yl}-2-(2-methylphenyl)-3(2H)-pyridazinone was obtained according to a similar manner to Example 1.

Mass ESI (+) 471 (M+1)

¹H-NMR (500 MHz, CDCl₃) δ 2.22 (3H, s), 2.24 (6H, s), 2.27-2.39 (3H, m), 3.09 (1H, dd, J=9.0, 9.0 Hz), 3.46 (1H, d, J=11.4 Hz), 3.81 (1H, dd, J=7.3, 12.4 Hz), 3.84 (3H, s), 4.26 (1H, dd, J=4.5, 12.3 Hz), 5.84 (1H, brs), 6.78 (1H, d, J=10.2 Hz), 6.96-6.98 (1H, m), 7.08-7.10 (3H, m), 7.33-7.37 (5H, m)

### EXAMPLE 24

6-{2-(2,4-Difluorophenyl)-6-[(methylamino)methyl]-4,5,6,7-tetrahydropyrazolo[1,5-a]pyrimidin-3-yl}-2-(2-methylphenyl)-3(2H)-pyridazinone was obtained according to a similar manner to Example 1.

Mass ESI (+) 463 (M+1)

¹H-NMR (500 MHz, CDCl₃) δ 2.20 (3H, s), 2.49 (3H, s), 2.50-2.53 (1H, m), 2.74-2.79 (2H, m), 3.16-3.21 (1H, m), 3.48 (1H, d, J=11.5 Hz), 3.92 (1H, dd, J=7.4, 12.4 Hz), 4.29 (1H, dd, J=5.0, 12.4 Hz), 5.90 (1H, brs), 6.84 (1H, d, J=10.0 Hz), 6.92-7.03 (4H, m), 7.32-7.39 (4H, m), 7.53 (1H, dd, J=8.7, 15.0 Hz)

### EXAMPLE 25

6-{6-[(Dimethylamino)methyl]-2-(3,5-dimethylphenyl)-4,5,6,7-tetrahydropyrazolo[1,5-a]pyrimidin-3-yl}-2-(2-methylphenyl)-3(2H)-pyridazinone was obtained according to a similar manner to Example 1.

Mass ESI (+) 469 (M+1)

¹H-NMR (500 MHz, CDCl₃) δ 2.23 (9H, s), 2.26-2.29 (1H, m), 2.35 (6H, s), 2.37-2.43 (2H, m), 3.08 (1H, dd, J=9.2, 9.2 Hz), 3.46 (1H, d, J=11.9 Hz), 3.80 (1H, dd, J=7.8, 12.4 Hz), 4.25 (1H, dd, J=5.0, 12.8 Hz), 5.83 (1H, brs), 6.77 (1H, d, J=9.7 Hz), 7.05 (1H, s), 7.10-7.13 (3H, m), 7.34-7.38 (4H, m)

### EXAMPLE 26

6-(2-(2-Chloro-4-fluorophenyl)-6-[(dimethylamino)methyl]-4,5,6,7-tetrahydropyrazolo[1,5-a]pyrimidin-3-yl}-2-(2-methylphenyl)-3(2H)-pyridazinone was obtained according to a similar manner to Example 1.

Mass ESI (+) 493 (M+1)

¹H-NMR (500 MHz, CDCl₃) δ 2.20 (3H, s), 2.25 (6H, s), 2.28-2.47 (3H, m), 3.10 (1H, t, J=9.6 Hz), 3.47-3.51 (1H, m), 3.81 (1H, dd, J=8.7, 11.8 Hz), 4.26 (1H, dd, J=4.6, 12.3 Hz), 5.95 (1H, brs), 6.79 (2H, d, J=1.4 Hz), 7.12 (1H, dt, J=2.8, 8.3 Hz), 7.26-7.28 (1H, m), 7.33-7.38 (4H, m), 7.50 (1H, dd, J=6.4, 8.6 Hz)

### EXAMPLE 27

6-{6-[(Dimethylamino)methyl]-2-(3-methylphenyl)-4,5,6,7-tetrahydropyrazolo[1,5-a]pyrimidin-3-yl}-2-(2-methylphenyl)-3(2H)-pyridazinone was obtained according to a similar manner to Example 1.

Mass ESI (+) 455 (M+1)

¹H-NMR (500 MHz, CDCl₃) δ 2.22 (3H, s), 2.23 (6H, s), 2.25-2.39 (3H, m), 2.40 (3H, s), 3.08 (1H, dd, J=9.6, 9.6 Hz), 3.46 (1H, d, J=11.5 Hz), 3.80 (1H, dd, J=7.7, 12.4 Hz), 4.25 (1H, dd, J=4.6, 12.3 Hz), 5.83 (1H, brs), 6.77 (1H, d, J=9.5 Hz), 7.08 (1H, d, J=9.6 Hz), 7.23-7.37 (8H, m)

### EXAMPLE 28

6-[2-(4-Fluorophenyl)-6-(4-morpholinylmethyl)-4,5,6,7-tetrahydropyrazolo[1,5-a]pyrimidin-3-yl]-2-(2-methylphenyl)-3(2H)-pyridazinone hydrochloride was obtained according to a similar manner to Example 5.

Mass ESI (+) 501 (M+1)

¹H-NMR (500 MHz, DMSO-d₆) δ 2.09 (3H, s), 2.72-2.82 (1H, m), 3.00-3.26 (5H, m), 3.38-4.06 (8H, m), 4.29-4.38 (1H, m), 6.11 (1H, brs), 6.95 (1H, d, J=9.5 Hz), 7.10 (1H, d, J=9.5 Hz), 7.25 (2H, dd, J=8.5, 18.5 Hz), 7.30-7.41 (4H, m), 7.49 (2H, dd, J=5.5, 8.5 Hz), 10.7 (1H, brs)

### EXAMPLE 29

6-[2-(4-Fluorophenyl)-6-[[(2-hydroxyethyl)(methyl)amino]methyl]-4,5,6,7-tetrahydropyrazolo[1,5-a]pyrimidin-3-yl]-2-(2-methylphenyl)-3(2H)-pyridazinone hydrochloride was obtained according to a similar manner to Example 5.

Mass ESI (+) 489 (M+1)

¹H-NMR (500 MHz, DMSO-d₆) δ 2.09 (3H, s), 2.67-2.76 (1H, m), 2.86 (3H, d, J=4.1 Hz), 3.09-3.21 (3H, m), 3.25-3.36 (2H, m), 3.42-3.54 (1H, m), 3.75-3.82 (2H, m), 3.92-4.00 (1H, m), 4.28-4.40 (1H, m), 6.13 (1H, brs), 6.95 (1H, d, J=19.5 Hz), 7.10 (1H, dd, J=2.6, 9.5 Hz), 7.25 (2H, dd, J=8.7, 8.7 Hz), 7.32-7.42 (4H, m), 7.49 (2H, dd, J=5.9, 8.7 Hz), 10.0 (1H, brs)

### EXAMPLE 30

6-[4-Ethyl-2-(4-fluorophenyl)-4,5,6,7-tetrahydropyrazolo[1,5-a]pyrimidin-3-yl]-2-(2-methylphenyl)-3(2H)-pyridazinone was obtained according to a similar manner to Example 6.

Mass ESI (+) 489 (M+1)

¹H-NMR (500 MHz, CDCl₃) δ 0.96 (3H, t, J=6.7 Hz), 2.15 (3H, s), 2.17-2.20 (2H, m), 3.21-3.25 (4H, m), 4.14 (2H, t, J=5.9 Hz), 6.92 (1H, d, J=9.6 Hz), 7.01 (2H, t, J=8.6 Hz), 7.14 (1H, d, J=9.6 Hz), 7.20 (1H, d, J=7.4 Hz), 7.29-7.36 (3H, m), 7.41 (2H, dd, J=5.5, 8.7 Hz)

### EXAMPLE 31

Ethyl 3-{6-(4-fluorophenyl)-7-(1-(2-methylphenyl)-6-oxo-1,6-dihydro-3-pyridazinyl]-2,3-dihydro-1H-imidazo[1,2-b]pyrazol-1-yl}propanoate was obtained according to a similar manner to Example 6.

Mass ESI (+) 488 (M+1)

¹H-NMR (500 MHz, CDCl₃) δ 1.21 (3H, t, J=7.3 Hz), 2.20 (3H, s), 2.34 (2H, t, J=6.9 Hz), 3.56 (2H, t, J=6.4 Hz), 3.86 (2H, t, J=8.7 Hz), 4.07 (2H, q, J=6.9 Hz), 4.19 (2H, t, J=7.9 Hz), 6.86 (1H, d, J=9.6 Hz), 6.96 (1H, d, J=9.6 Hz), 7.08 (2H, t, J=8.7 Hz), 7.29-7.36 (4H, m), 7.43 (2H, dd, J=5.5, 8.6 Hz)

### EXAMPLE 32

6-[2-(4-Fluorophenyl)-4-(3-hydroxypropyl)-4,5,6,7-tetrahydropyrazolo[1,5-a]pyrimidin-3-yl]-2-(2-methylphenyl)-3(2H)-pyridazinone was obtained according to a similar manner to Example 10.

Mass ESI (+) 460 (M+1)

¹H-NMR (CDCl₃) δ 1.59-1.64 (2H, m), 2.15-2.19 (5H, m), 3.25 (2H, t, J=5.5 Hz), 3.33 (2H, brs), 3.40-3.46 (3H, m), 4.14 (2H, t, J=6.3 Hz), 6.91 (1H, d, J=9.7 Hz), 7.01 (2H, dd, J=8.7, 8.7 Hz), 7.12 (1H, d, J=9.7 Hz), 7.23-7.36 (4H, m), 7.40 (2H, dd, J=5.5, 8.7 Hz).

### EXAMPLE 33

6-{6-[(Diethylamino)methyl]-2-(2,4-difluorophenyl)-4,5,6,7-tetrahydropyrazolo[1,5-a]pyrimidin-3-yl}-2-(2-methylphenyl)pyridazin-3(2H)-one was obtained according to a similar manner to Example 1.

Mass ESI (+) 506 (M+1)

¹H-NMR (CDCl₃) δ 0.98 (6H, t, J=7.4 Hz), 2.21 (3H, s), 2.37-2.43 (3H, m), 4.50 (4H, q, J=7.34 Hz), 3.07 (1H, dd, J=9.2, 9.2 Hz), 3.47 (1H, d, J=11.5 Hz), 3.82 (1H, dd, J=9.2, 9.2 Hz), 4.26 (1H, dd, J=4.1, 12.4 Hz), 5.89 (1H, brs), 6.83 (1H, d, J=9.5 Hz), 6.92-6.97 (m, 2H), 7.01 (1H, ddd, J=2.5, 8.3, 8.3 Hz), 7.34-7.39 (4H, m), 7.54 (1H, dd, J=8.3, 15.2 Hz)

### EXAMPLE 34

6-[6-{[Benzyl(methyl)amino]methyl}-2-(3-methylphenyl)-4,5,6,7-tetrahydropyrazolo[1,5-a]pyrimidin-3-yl]-2-(2-methylphenyl)pyridazin-3(2H)-one was obtained according to a similar manner to Example 1.

Mass ESI (+) 531 (M+1)

¹H-NMR (CDCl₃) δ 2.23 (3H, s), 2.24 (3H, s), 2.38-2.42 (5H, m), 2.47-2.51 (1H, m), 3.01 (1H, dd, J=9.7, 9.7 Hz), 3.46-3.55 (3H, m), 3.76 (1H, dd, J=9.6, 13.3 Hz), 4.31 (1H, dd, J=3.7, 13.3 Hz), 5.79 (1H, brs), 6.77 (1H, d, J=9.9 Hz), 7.08 (1H, d, J=10.0 Hz), 7.23-7.38 (13H, m)

### EXAMPLE 35

6-[2-(4-Fluorophenyl)-6,6-bis(hydroxymethyl)-4,5,6,7-tetrahydropyrazolo[1,5-a]pyrimidin-3-yl)-2-(2-methylphenyl)pyridazin-3(2H)-one was obtained according to a similar manner to Example 1.

Mass ESI (+) 462 (M+1)

¹H-NMR (CDCl₃) δ 2.22 (3H, s), 3.28 (2H, s), 3.77 (4H, m), 3.99 (2H, s), 5.80 (1H, brs), 6.80 (1H, d, J=10.1 Hz), 7.02 (1H, d, J=10.1 Hz), 7.15 (2H, dd, J=8.7 Hz, J=8.7 Hz), 7.31-7.41 (4H, m), 7.48 (2H, dd, J=8.7 Hz, J=5.5 Hz)

### EXAMPLE 36

6-{6-[(Dibenzylamino)methyl]-2-(2,4-difluorophenyl)-4,5,6,7-tetrahydropyrazolo[1,5-a]pyrimidin-3-yl}-2-(2-methylphenyl)pyridazin-3(2H)-one was obtained according to a similar manner to Example 1.

Mass ESI (+) 629 (M+1)

¹H-NMR (CDCl₃) δ 2.20 (3H, s), 2.46 (2H, d, J=7.7 Hz), 2.51-2.57 (1H, m), 2.83-2.89 (1H, m), 3.41-3.45 (1H, m), 3.49 (2H, d, J=13.8 Hz), 3.61-3.68 (3H, m), 4.36 (1H, dd, J=5.2, 12.4 Hz), 5.78 (1H, brs), 6.82 (1H, d, J=10.1 Hz), δ 6.92-6.96 (2H, m), 7.00-7.04 (1H, m), 7.21-7.39 (14H, m), 7.51-7.55 (1H, m)

### EXAMPLE 37

6-[2-(2,4-Difluorophenyl)-6-(hydroxymethyl)-4,5,6,7-tetrahydropyrazolo[1,5-a]pyrimidin-3-yl]-2-(2-methylphenyl)pyridazin-3(2H)-one was obtained according to a similar manner to Example 1.

Mass ESI (+) 450 (M+1)

¹H-NMR (CDCl₃) δ 2.21 (3H, s), 2.44-2.49 (1H, m), 3.24 (1H, dd, J=9.6, 9.6 Hz), 3.46 (1H, d, J=12.2 Hz), 3.70-3.76 (2H, m), 3.98 (1H, dd, J=7.5, 12.9 Hz), 4.26 (1H, dd, J=4.9, 12.4 Hz), 5.88 (1H, brs), 6.83 (1H, d, J=10.0 Hz), 6.92-6.97 (2H, m), 7.02 (1H, ddd, J=1.9, 8.7, 8.7 Hz), 7.29-7.36 (4H, m), 7.54 (1H, dd, J=8.7, 14.9 Hz)

### EXAMPLE 38

6-[6-(Hydroxymethyl)-2-(3-methylphenyl)-4,5,6,7-tetrahydropyrazolo[1,5-a]pyrimidin-3-yl]-2-(2-methylphenyl)pyridazin-3(2H)-one was obtained according to a similar manner to Example 1.

Mass ESI (+) 428 (M+1)

¹H-NMR (CDCl₃) δ 2.23 (3H, s), 2.40 (3H, s), 2.42-2.46 (1H, m), 3.22 (1H, dd, J=9.7, 9.7 Hz), 3.45 (1H, d, J=11.1 Hz), 3.67-3.75 (2H, m), 3.97 (1H, dd, J=7.4, 12.3 Hz), 4.24 (1H, dd, J=4.6, 12.2 Hz), 5.81 (1H, brs), 6.77 (1H, d, J=10.1 Hz), 7.08 (1H, d, J=9.8 Hz), 7.22-7.37 (8H, m)

### EXAMPLE 39

6-[6-{[Benzyl(tert-butyl)amino]methyl)-2-(2,4-difluorophenyl)-4,5,6,7-tetrahydropyrazolo[1,5-a]pyrimidin-3-yl]-2-(2-methylphenyl)pyridazin-3(2H)-one was obtained according to a similar manner to Example 1.

Mass ESI (+) 595 (M+1)

¹H-NMR (CDCl₃) δ 1.92 (9H, s), 2.20 (3H, s), 2.54-2.58 (1H, m), 2.64-2.69 (1H, m), 2.85-2.90 (1H, m), 3.25-3.29 (1H, m), 3.66 (1H, d, J=15.7 Hz), 3.78-3.82 (2H, m), 4.05 (1H, dd, J=4.6, 12.4 Hz), 5.72 (1H, brs), 6.81 (1H, d, J=9.8 Hz), 6.90-6.95 (2H, m), 7.00 (1H, ddd, J=2.3, 8.4, 8.4 Hz), 7.17 (1H, dd, J=7.3, 7.3 Hz), 7.23-7.27 (2H, m), 7.30-7.39 (6H, m), 7.51 (1H, dd, J=8.2, 14.5 Hz)

### EXAMPLE 40

6-[2-(2-Chloro-4-fluorophenyl)-6,6-bis(hydroxymethyl)-4,5,6,7-tetrahydropyrazolo[1,5-a]pyrimidin-3-yl]-2-(2-methylphenyl)pyridazin-3(2H)-one was obtained according to a similar manner to Example 1.

Mass ESI (−) 494 (M−1)

¹H-NMR (CDCl₃) δ 2.20 (3H, s), 2.27 (2H, m), 3.30 (2H, s), 3.77 (4H, m), 4.01 (2H, s), 5.92 (1H, brs), 6.78 (1H, d, 10.1 Hz), 6.81 (1H, d, J=10.1 Hz), 7.12 (1H, ddd, J=2.8 Hz, J=8.2 Hz, J=8.2 Hz), 7.26 (1H, dd, J=2.8 Hz, J=8.2 Hz), 7.31-7.41 (4H, m), 7.49 (1H, dd, J=8.2 Hz, J=6.0 Hz)

### EXAMPLE 41

6-[2-(2,4-Difluorophenyl)-6,6-bis(hydroxymethyl)-4,5,6,7-tetrahydropyrazolo[1,5-a]pyrimidin-3-yl]-2-(2-methylphenyl)pyridazin-3(2H)-one was obtained according to a similar manner to Example 1.

Mass ESI (−) 478 (M−1)

¹H-NMR (CDCl₃) δ 2.21 (3H, s), 2.22 (2H, t, J=4.6 Hz), 3.28 (2H, s), 3.76 (4H, d, J=4.6 Hz), 4.01 (2H, s), 5.87 (1H, brs), 6.83 (1H, d, J=9.6 Hz), 6.94 (1H, ddd, J=2.7 Hz, J=9.6 Hz, J=9.6 Hz), 6.96 (1H, dd, J=1.8 Hz, J=8.7 Hz), 7.01 (1H, ddd, J=2.3 Hz, J=6.4 Hz, J=8.7 Hz), 7.31-7.42 (4H, m), 7.54 (1H, dt, J=6.4 Hz, J=8.7 Hz)

### EXAMPLE 42

6-[6,6-Bis(hydroxymethyl)-2-(3-methylphenyl)-4,5,6,7-tetrahydropyrazolo[1,5-a]pyrimidin-3-yl]-2-(2-methylphenyl)pyridazin-3(2H)-one was obtained according to a similar manner to Example 1.

Mass ESI (+) 458 (M+1)

¹H-NMR (CDCl₃) δ 2.22 (3H, s), 2.39 (3H, s), 3.22 (2H, s), 3.69 (4H, s), 3.98 (2H, s), 5.80 (1H, brs), 6.77 (1H, d, J=9.7 Hz), 7.06 (1H, d, J=9.6 Hz), 7.22-7.26 (2H, m), 7.30-7.39 (6H, m)

### EXAMPLE 43

{2-(3-Methylphenyl)-3-[1-(2-methylphenyl)-6-oxo-1,6-dihydropyridazin-3-yl]-4,5,6,7-tetrahydropyrazolo[1,5-a]pyrimidine-6,6-diyl}bis(methylene) diacetate was obtained according to a similar manner to Example 1.

¹H-NMR (CDCl₃) δ 2.06 (6H, s), 2.22 (3H, s), 2.40 (3H, s), 3.29 (2H, s), 4.07 (2H, s), 4.14 (4H, dd, J=11.5, 22.8 Hz), 5.84 (1H, brs), 6.77 (1H, d, J=10.3 Hz), 7.08 (1H, d, J=10.2 Hz), 7.23-7.29 (2H, m), 7.31-7.37 (6H, m)

### EXAMPLE 44

{6-(Hydroxymethyl)-2-(3-methylphenyl)-3-[1-(2-methylphenyl)-6-oxo-1,6-dihydropyridazin-3-yl]-4,5,6,7-tetrahydropyrazolo[1,5-a]pyrimidin-6-yl}methyl acetate was obtained according to a similar manner to Example 1.

¹H-NMR (CDCl₃) δ 2.11 (3H, s), 2.23 (3H, s), 2.40 (3H, s), 3.27 (2H, s), 3.57 (2H, s), 4.01 (2H, dd, J=13.2, 37.0 Hz), 4.21 (2H, dd, J=11.6, 45.5 Hz), 5.82 (1H, brs), 6.78 (1H, d, J=9.6 Hz), 7.08 (1H, d, J=9.5 Hz), 7.23-7.28 (2H, m), 7.32-7.40 (6H, m)

### EXAMPLE 45

6-[(6Z)-2-(2,4-Difluorophenyl)-6-(2-hydroxyethylidene)-4,5,6,7-tetrahydropyrazolo[1,5-a]pyrimidin-3-yl]-2-(2-methylphenyl)pyridazin-3(2H)-one was obtained according to a similar manner to Example 1.

Mass ESI (+) 462 (M+1)

¹H-NMR (CDCl₃) δ 2.20 (3H, s), 3.87 (2H, s), 4.27 (2H, d, J=6.4 Hz), 4.85 (2H, s), 5.86 (1H, t, J=6.4 Hz), 5.95 (1H, brs), 6.83 (1H, d, J=10.1 Hz), 6.93-7.04 (3H, m), 7.32-7.38 (4H, m), 7.53 (1H, dd, J=8.2, 15.1 Hz)

### EXAMPLE 46

6-{2-(4-Fluorophenyl)-6-[(3-hydroxyazetidin-1-yl)methyl]-4,5,6,7-tetrahydropyrazolo[1,5-a]pyrimidin-3-yl}-2-(2-methylphenyl)pyridazin-3(2H)-one was obtained according to a similar manner to Example 5.

Mass ESI (+) 487 (M+1)

¹H NMR (CDCl₃) δ 1.98 (1H, brs), 2.22 (3H, s), 2.24 (1H, s), 2.48-2.62 (2H, m), 2.86-2.99 (2H, m), 3.05-3.15 (1H, m), 3.40-3.47 (1H, m), 3.63-3.74 (2H, m), 3.83 (1H, dd, J=8.2 Hz, J=12.6 Hz), 4.21 (1H, dd, J=12.6 Hz, J=5.0 Hz), 4.39-4.47 (1H, m), 5.80 (1H, brs), 6.79 (1H, d, J=10.1 Hz), 7.01 (1H, d, J=10.1 Hz), 7.14 (2H, dd, J=8.7 Hz, J=8.7 Hz), 7.31-7.41 (4H, m), 7.49 (2H, dd, J=8.7 Hz, J=5.5 Hz)

### EXAMPLE 47

6-(2-(4-Fluorophenyl)-6-[(4-methylpiperazin-1-yl)methyl]-4,5,6,7-tetrahydropyrazolo[1,5-a]pyrimidin-3-yl)-2-(2-methylphenyl)pyridazin-3(2H)-one dihydrochloride was obtained according to a similar manner to Example 5.

Mass ESI (+) 514 (M+1)

¹H-NMR (DMSO-d₆) δ 2.09 (3H, s), 2.59-2.88 (4H, m), 3.04-4.07 (14H, m), 4.22-4.39 (1H, m), 6.95 (1H, d, J=10.3 Hz), 7.10 (1H, d, J=10.3 Hz), 7.21-7.39 (4H, m), 7.45-7.52 (2H, m)

### EXAMPLE 48

6-[2-(4-Fluorophenyl)-6-(pyrrolidin-1-ylmethyl)-4,5,6,7-tetrahydropyrazolo[1,5-a]pyrimidin-3-yl]-2-(2-methylphenyl)pyridazin-3(2H)-one hydrochloride was obtained according to a similar manner to Example 5.

Mass ESI (+) 485 (M+1)

¹H-NMR (DMSO-d₆) δ 1.83-2.05 (4H, m), 2.09 (3H, s), 2.59-2.69 (1H, m), 2.95-3.08 (2H, m), 3.12-3.27 (3H, m), 3.46-3.52 (1H, m), 3.57-3.66 (2H, m), 3.94-4.00 (1H, m), 4.29-4.36 (1H, m), 6.12 (1H, brs), 6.95 (1H, d, J=9.8 Hz), 7.10 (1H, d, J=9.8 Hz), 7.21-7.29 (2H, m), 7.30-7.40 (4H, m), 7.46-7.52 (2H, m), 10.48 (1H, brs)

### EXAMPLE 49

6-[2-(4-Fluorophenyl)-6-{[(2-methoxyethyl)amino]methyl}-4,5,6,7-tetrahydropyrazolo[1,5-a]pyrimidin-3-yl]-2-(2-methylphenyl)pyridazin-3(2H)-one was obtained according to a similar manner to Example 5.

Mass ESI (+) 489 (M+H)

¹H-NMR (CDCl₃) δ 2.22 (3H, s), 2.35-2.43 (1H, m), 2.70-2.72 (2H, m), 2.75-2.80 (2H, m), 3.08-3.19 (1H, m), 3.35 (3H, s), 3.42-3.51 (3H, m), 3.83-3.90 (1H, m), 4.21-4.30 (1H, m), 5.81 (1H, s), 6.79 (1H, d, J=9.5 Hz), 7.01 (1H, d, J=10.0 Hz), 7.10-7.18 (2H, m), 7.30-7.40 (4H, m), 7.48-7.52 (2H, m)

### EXAMPLE 50

2-(2-Methylphenyl)-6-(2-phenylpyrazolo[1,5-a]pyrazin-3-yl)pyridazin-3(2H)-one was obtained according to a similar manner to Example 15.

Mass ESI (+) 380 (M+1)

¹H-NMR (DMSO-d₆) δ 2.16 (3H, s), 7.10 (1H, d, J=9.9 Hz), 7.34-7.43 (4H, m), 7.44-7.48 (1H, m), 7.51-7.57 (3H, m), 7.67-7.72 (2H, m), 8.07 (1H, d, J=4.9 Hz), 8.92 (1H, dd, J=1.4, 4.9 Hz), 9.23 (1H, d, J=1.4 Hz)

### EXAMPLES 51 AND 52

A racemate, 6-{2-(2,4-difluorophenyl)-6-[(dimethylamino)methyl]-4,5,6,7-tetrahydropyrazolo[1,5-a]pyrimidin-3-yl}-2-(2-methylphenyl)pyridazin-3(2H)-one was separated into each optical isomer, (+)-6-{2-(2,4-difluorophenyl)-6-[(dimethylamino)methyl]-4,5,6,7-tetrahydropyrazolo[1,5-a]pyrimidin-3-yl}-2-(2-methylphenyl)pyridazin-3(2H)-one and (−)-6-{2-(2,4-difluorophenyl)-6-[(dimethylamino)methyl]-4,5,6,7-tetrahydropyrazolo[1,5-a]pyrimidin-3-yl}-2-(2-methylphenyl)pyridazin-3(2H)-one by using of chiral HPLC method.
(+)-6-{2-(2,4-Difluorophenyl)-6-[(dimethylamino)methyl]-4,5,6,7-tetrahydropyrazolo[1,5-a]pyrimidin-3-yl}-2-(2-methylphenyl)pyridazin-3(2H)-one (Example 51)

[α]²⁶_{D}=+313° (c=1.0, CHCl₃)
(−)-6-{2-(2,4-Difluorophenyl)-6-[(dimethylamino)methyl]-4,5,6,7-tetrahydropyrazolo[1,5-a]pyrimidin-3-yl}-2-(2-methylphenyl)pyridazin-3(2H)-one (Example 52)

[α]²⁶_{D}=−314° (c=1.0, CHCl₃)

### EXAMPLES 53 AND 54

(+)-6-[2-(4-Fluorophenyl)-6-(hydroxymethyl)-4,5,6,7-tetrahydropyrazolo[1,5-a]pyrimidin-3-yl]-2-(2-methylphenyl)pyridazin-3(2H)-one and (−)-6-[2-(4-fluorophenyl)-6-(hydroxymethyl)-4,5,6,7-tetrahydropyrazolo[1,5-a]pyrimidin-3-yl]-2-(2-methylphenyl)pyridazin-3(2H)-one were obtained according to a similar manner to Examples 51 and 52.
(+)-6-[2-(4-Fluorophenyl)-6-(hydroxymethyl)-4,5,6,7-tetrahydropyrazolo[1,5-a]pyrimidin-3-yl]-2-(2-methylphenyl)pyridazin-3(2H)-one (Example 53)

[α]²⁶_{D}=−50.3° (c=0.45, CHCl₃)
(−)-6-[2-(4-Fluorophenyl)-6-(hydroxymethyl)-4,5,6,7-tetrahydropyrazolo[1,5-a]pyrimidin-3-yl]-2-(2-methylphenyl)pyridazin-3(2H)-one (Example 54)

[α]²⁶_{D}=50.3° (c=0.98, CHCl₃)

The following optical isomers could be also obtained in a similar manner to Examples 51 and 52.
(+)-6-{2-(4-Fluorophenyl)-6-[(dimethylamino)methyl]-4,5,6,7-tetrahydropyrazolo[1,5-a]pyrimidin-3-yl}-2-(2-methylphenyl)pyridazin-3(2H)-one
(−)-6-{2-(4-Fluorophenyl)-6-[(dimethylamino)methyl]-4,5,6,7-tetrahydropyrazolo[1,5-a]pyrimidin-3-yl}-2-(2-methylphenyl)pyridazin-3(2H)-one
(+)-6-{2-(3-Methylphenyl)-6-[(dimethylamino)methyl]-4,5,6,7-tetrahydropyrazolo[1,5-a]pyrimidin-3-yl}-2-(2-methylphenyl)pyridazin-3(2H)-one
(−)-6-(2-(3-Methylphenyl)-6-[(dimethylamino)methyl)-4,5,6,7-tetrahydropyrazolo[1,5-a]pyrimidin-3-yl)-2-(2-methylphenyl)pyridazin-3(2H)-one
(+)-6-{2-(2-Chloro-4-fluorophenyl)-6-[(dimethylamino)methyl]-4,5,6,7-tetrahydropyrazolo[1,5-a]pyrimidin-3-yl}-2-(2-methylphenyl)pyridazin-3(2H)-one
(−)-6-{2-(2-Chloro-4-fluorophenyl)-6-[(dimethylamino)methyl]-4,5,6,7-tetrahydropyrazolo[1,5-a]pyrimidin-3-yl}-2-(2-methylphenyl)pyridazin-3(2H)-one
(+)-6-{2,5-Difluorophenyl)-6-[(dimethylamino)methyl]-4,5,6,7-tetrahydropyrazolo[1,5-a]pyrimidin-3-yl}-2-(2-methylphenyl)pyridazin-3(2H)-one
(−)-6-(2-(2,5-Difluorophenyl)-6-[(dimethylamino)methyl]-4,5,6,7-tetrahydropyrazolo[1,5-a]pyrimidin-3-yl)-2-(2-methylphenyl)pyridazin-3(2H)-one
(+)-6-{2-(2,4-Difluorophenyl)-6-[(diethylamino)methyl]-4,5,6,7-tetrahydropyrazolo[1,5-a]pyrimidin-3-yl}-2-(2-methylphenyl)pyridazin-3(2H)-one
(−)-6-{2-(2,4-Difluorophenyl)-6-[(diethylamino)methyl]-4,5,6,7-tetrahydropyrazolo[1,5-a]pyrimidin-3-yl}-2-(2-methylphenyl)pyridazin-3(2H)-one
(+)-6-{2-(4-Fluorophenyl)-6-[(diethylamino)methyl]-4,5,6,7-tetrahydropyrazolo[1,5-a]pyrimidin-3-yl}-2-(2-methylphenyl)pyridazin-3(2H)-one
(−)-6-{2-(4-Fluorophenyl)-6-[(diethylamino)methyl]-4,5,6,7-tetrahydropyrazolo[1,5-a]pyrimidin-3-yl}-2-(2-methylphenyl)pyridazin-3(2H)-one
(+)-6-{2-(3-Methylphenyl)-6-[(dimethylamino)methyl]-4,5,6,7-tetrahydropyrazolo[1,5-a]pyrimidin-3-yl}-2-(2-methylphenyl)pyridazin-3(2H)-one
(−)-6-{2-(3-Methylphenyl)-6-[(diethylamino)methyl]-4,5,6,7-tetrahydropyrazolo[1,5-a]pyrimidin-3-yl}-2-(2-methylphenyl)pyridazin-3(2H)-one
(+)-6-{2-(2-Chloro-4-fluorophenyl)-6-[(diethylamino)methyl]-4,5,6,7-tetrahydropyrazolo[1,5-a]pyrimidin-3-yl}-2-(2-methylphenyl)pyridazin-3(2H)-one
(−)-6-{2-(2-Chloro-4-fluorophenyl)-6-[(diethylamino)methyl]-4,5,6,7-tetrahydropyrazolo[1,5-a]pyrimidin-3-yl}-2-(2-methylphenyl)pyridazin-3(2H)-one
(+)-6-{2,5-Difluorophenyl)-6-[(diethylamino)methyl)-4,5,6,7-tetrahydropyrazolo[1,5-a]pyrimidin-3-yl}-2-(2-methylphenyl)pyridazin-3(2H)-one
(−)-6-{2-(2,5-Difluorophenyl)-6-[(diethylamino)methyl]-4,5,6,7-tetrahydropyrazolo[1,5-a]pyrimidin-3-yl}-2-(2-methylphenyl)pyridazin-3(2H)-one
(+)-6-(2-(2,4-Difluorophenyl)-6-(hydroxymethyl)-4,5,6,7-tetrahydropyrazolo[1,5-a]pyrimidin-3-yl]-2-(2-methylphenyl)pyridazin-3(2H)-one
(−)-6-[2-(2,4-Difluorophenyl)-6-(hydroxymethyl)-4,5,6,7-tetrahydropyrazolo[1,5-a]pyrimidin-3-yl]-2-(2-methylphenyl)pyridazin-3(2H)-one
(+)-6-[2-(3-Methylphenyl)-6-(hydroxymethyl)-4,5,6,7-tetrahydropyrazolo[1,5-a]pyrimidin-3-yl]-2-(2-methylphenyl)pyridazin-3(2H)-one
(−)-6-[2-(3-Methylphenyl)-6-(hydroxymethyl)-4,5,6,7-tetrahydropyrazolo[1,5-a]pyrimidin-3-yl]-2-(2-methylphenyl)pyridazin-3(2H)-one
(+)-6-[2-(2,5-Difluorophenyl)-6-(hydroxymethyl)-4,5,6,7-tetrahydropyrazolo[1,5-a]pyrimidin-3-yl]-2-(2-methylphenyl)pyridazin-3(2H)-one
(−)-6-[2-(2,5-Difluorophenyl)-6-(hydroxymethyl)-4,5,6,7-tetrahydropyrazolol[1,5-a]pyrimidin-3-yl]-2-(2-methylphenyl)pyridazin-3(2H)-one
(+)-6-[2-(2-Chloro-4-fluorophenyl)-6-(hydroxymethyl)-4,5,6,7-tetrahydropyrazolo[1,5-a]pyrimidin-3-yl]-2-(2-methylphenyl)pyridazin-3(2H)-one
(−)-6-[2-(2-Chloro-4-fluorophenyl)-6-(hydroxymethyl)-4,5,6,7-tetrahydropyrazolo[1,5-a]pyrimidin-3-yl]-2-(2-methylphenyl)pyridazin-3(2H)-one
(+)-6-{2-(4-Fluorophenyl)-6-[(methylamino)methyl]-4,5,6,7-tetrahydropyrazolo[1,5-a]pyrimidin-3-yl}-2-(2-methylphenyl)pyridazin-3(2H)-one
(−)-6-{2-(4-Fluorophenyl)-6-[(methylamino)methyl]-4,5,6,7-tetrahydropyrazolo[1,5-a]pyrimidin-3-yl}-2-(2-methylphenyl)pyridazin-3(2H)-one
(+)-6-{2-(2,4-Difluorophenyl)-6-[(methylamino)methyl]-4,5,6,7-tetrahydropyrazolo[1,5-a]pyrimidin-3-yl}-2-(2-methylphenyl)pyridazin-3(2H)-one
(−)-6-{2-(2,4-Difluorophenyl)-6-[(methylamino)methyl]-4,5,6,7-tetrahydropyrazolo[1,5-a]pyrimidin-3-yl}-2-(2-methylphenyl)pyridazin-3(2H)-one
(+)-6-{2-(2,5-Difluorophenyl)-6-[(methylamino)methyl]-4,5,6,7-tetrahydropyrazolo[1,5-a]pyrimidin-3-yl}-2-(2-methylphenyl)pyridazin-3(2H)-one
(−)-6-{2-(2,5-Difluorophenyl)-6-[(methylamino)methyl]-4,5,6,7-tetrahydropyrazolo[1,5-a]pyrimidin-3-yl}-2-(2-methylphenyl)pyridazin-3(2H)-one
(+)-6-{2-(3-Methylphenyl)-6-[(methylamino)methyl]-4,5,6,7-tetrahydropyrazolo[1,5-a]pyrimidin-3-yl}-2-(2-methylphenyl)pyridazin-3(2H)-one
(−)-6-{2-(3-Methylphenyl)-6-[(methylamino)methyl)-4,5,6,7-tetrahydropyrazolo[1,5-a]pyrimidin-3-yl}-2-(2-methylphenyl)pyridazin-3(2H)-one
(+)-6-{2-(2-Chloro-4-fluorophenyl)-6-[(methylamino)methyl]-4,5,6,7-tetrahydropyrazolo[1,5-a]pyrimidin-3-yl}-2-(2-methylphenyl)pyridazin-3(2H)-one
(−)-6-{2-(2-Chloro-4-fluorophenyl)-6-[(methylamino)methyl]-4,5,6,7-tetrahydropyrazolo[1,5-a]pyrimidin-3-yl}-2-(2-methylphenyl)pyridazin-3(2H)-one
(+)-6-{6-[(tert-Butylamino)methyl]-2-(4-fluorophenyl)-4,5,6,7-tetrahydropyrazolo[1,5-a]pyrimidin-3-yl}-2-(2-methylphenyl)pyridazin-3(2H)-one
(−)-6-{6-[(tert-Butylamino)methyl]-2-(4-fluorophenyl)-4,5,6,7-tetrahydropyrazolo[1,5-a]pyrimidin-3-yl}-2-(2-methylphenyl)pyridazin-3(2H)-one
(+)-6-{6-[(tert-Butylamino)methyl]-2-(2,4-difluorophenyl)-4,5,6,7-tetrahydropyrazolo[1,5-a]pyrimidin-3-yl}-2-(2-methylphenyl)pyridazin-3(2H)-one
(−)-6-{6-[(tert-Butylamino)methyl]-2-(2,4-difluorophenyl)-4,5,6,7-tetrahydropyrazolo[1,5-a]pyrimidin-3-yl}-2-(2-methylphenyl)pyridazin-3(2H)-one
(+)-6-{6-[(tert-Butylamino)methyl]-2-(2,5-difluorophenyl)-4,5,6,7-tetrahydropyrazolo[1,5-a]pyrimidin-3-yl}-2-(2-methylphenyl)pyridazin-3(2H)-one
(−)-6-(6-[(tert-Butylamino)methyl]-2-(2,5-difluorophenyl)-4,5,6,7-tetrahydropyrazolo[1,5-a]pyrimidin-3-yl}-2-(2-methylphenyl)pyridazin-3(2H)-one
(+)-6-{6-[(tert-Butylamino)methyl]-2-(3-methylphenyl)-4,5,6,7-tetrahydropyrazolo[1,5-a]pyrimidin-3-yl}-2-(2-methylphenyl)pyridazin-3(2H)-one
(−)-6-{6-[(tert-Butylamino)methyl]-2-(3-methylphenyl)-4,5,6,7-tetrahydropyrazolo[1,5-a]pyrimidin-3-yl}-2-(2-methylphenyl)pyridazin-3(2H)-one
(+)-6-{2-(4-Fluorophenyl)-6-[(4-methylpiperazin-1-yl)methyl]-4,5,6,7-tetrahydropyrazolo[1,5-a]pyrimidin-3-yl}-2-(2-methylphenyl)pyridazin-3(2H)-one
(−)-6-{2-(4-Fluorophenyl)-6-[(4-methylpiperazin-1-yl)methyl]-4,5,6,7-tetrahydropyrazolo[1,5-a]pyrimidin-3-yl}-2-(2-methylphenyl)pyridazin-3(2H)-one
(+)-6-{2-(2,4-Difluorophenyl)-6-[(4-methylpiperazin-1-yl)methyl]-4,5,6,7-tetrahydropyrazolo[1,5-a]pyrimidin-3-yl}-2-(2-methylphenyl)pyridazin-3(2H)-one
(−)-6-{2-(2,4-Difluorophenyl)-6-[(4-methylpiperazin-1-yl)methyl]-4,5,6,7-tetrahydropyrazolo[1,5-a]pyrimidin-3-yl}-2-(2-methylphenyl)pyridazin-3(2H)-one
(+)-6-{2-(2,5-Difluorophenyl)-6-[(4-methylpiperazin-1-yl)methyl]-4,5,6,7-tetrahydropyrazolo[1,5-a]pyrimidin-3-yl}-2-(2-methylphenyl)pyridazin-3(2H)-one
(−)-6-{2-(2,5-Difluorophenyl)-6-[(4-methylpiperazin-1-yl)methyl]-4,5,6,7-tetrahydropyrazolo[1,5-a]pyrimidin-3-yl}-2-(2-methylphenyl)pyridazin-3(2H)-one
(+)-6-{2-(3-Methylphenyl)-6-[(4-methylpiperazin-1-yl)methyl]-4,5,6,7-tetrahydropyrazolo[1,5-a]pyrimidin-3-yl}-2-(2-methylphenyl)pyridazin-3(2H)-one
(−)-6-{2-(3-Methylphenyl)-6-[(4-methylpiperazin-1-yl)methyl]-4,5,6,7-tetrahydropyrazolo[1,5-a]pyrimidin-3-yl}-2-(2-methylphenyl)pyridazin-3(2H)-one
(+)-2-(4-Fluorophenyl)-3-[1-(2-methylphenyl)-6-oxo-1,6-dihydropyridazin-3-yl]-4,5,6,7-tetrahydropyrazolo[1,5-a]pyrimidine-6-carbonitrile
(−)-2-(4-Fluorophenyl)-3-[1-(2-methylphenyl)-6-oxo-1,6-dihydropyridazin-3-yl]-4,5,6,7-tetrahydropyrazolo[1,5-a]pyrimidine-6-carbonitrile
(+)-2-(2,4-Difluorophenyl)-3-[1-(2-methylphenyl)-6-oxo-1,6-dihydropyridazin-3-yl]-4,5,6,7-tetrahydropyrazolo[1,5-a]pyrimidine-6-carbonitrile
(−)-2-(2,4-Difluorophenyl)-3-[1-(2-methylphenyl)-6-oxo-1,6-dihydropyridazin-3-yl]-4,5,6,7-tetrahydropyrazolo[1,5-a]pyrimidine-6-carbonitrile
(+)-2-(2,5-Difluorophenyl)-3-[1-(2-methylphenyl)-6-oxo-1,6-dihydropyridazin-3-yl]-4,5,6,7-tetrahydropyrazolo[1,5-a]pyrimidine-6-carbonitrile (−)-2-(2,5-Difluorophenyl)-3-[1-(2-methylphenyl)-6-oxo-1,6-dihydropyridazin-3-yl]-4,5,6,7-tetrahydropyrazolo[1,5-a]pyrimidine-6-carbonitrile
(+)-2-(3-Methylphenyl)-3-[1-(2-methylphenyl)-6-oxo-1,6-dihydropyridazin-3-yl]-4,5,6,7-tetrahydropyrazolo[1,5-a]pyrimidine-6-carbonitrile
(−)-2-(3-Methylphenyl)-3-[1-(2-methylphenyl)-6-oxo-1,6-dihydropyridazin-3-yl]-4,5,6,7-tetrahydropyrazolo[1,5-a]pyrimidine-6-carbonitrile

### EXAMPLE 55

To a suspension 6-{2-(2,4-difluorophenyl)-6-[(dimethylamino)methyl]-4,5,6,7-tetrahydropyrazolo[1,5-a]pyrimidin-3-yl}-2-(2-methylphenyl)pyridazin-3(2H)-one (125 mg) and zinc (17.1 mg) in acetic acid was stirred at 120° C. for 4 hours. The reaction mixture was cooled to rt and adjusted pH 9 with saturated aqueous NaHCO₃ solution. The whole mixture was extracted with ethyl acetate and THF. The organic phase was washed with brine, dried over Na₂SO₄, filtered and evaporated in vacuo. The thus obtained oil was triturated with hexane to give 6-{2-(2,4-difluorophenyl)-6-[(dimethylamino)methyl]-4,5,6,7-tetrahydropyrazolo[1,5-a]pyrimidin-3-yl}-2-(2-methylphenyl)-4,5-dihydropyridazin-3(2H)-one (121 mg).

Mass ESI (+) 479 (M+1)

¹H-NMR (DMSO-d₆) δ 2.14 (3H, s), 2.17 (6H, s), 2.22 (2H, m), 2.30 (1H, m), 2.43 (4H, m), 2.99 (1H, m), 3.39 (1H, m), 3.72 (1H, m), 4.09 (1H, m), 6.14 (1H, brs), 7.16 (1H, m), 7.25 (4H, m), 7.35 (1H, m), 7.50 (1H, m)

### EXAMPLE 56

A mixture of ethyl 2-(4-fluorophenyl)-3-[1-(2-methylphenyl)-6-oxo-1,6-dihydropyridazin-3-yl]-4,5,6,7-tetrahydropyrazolo[1,5-a]pyrimidine-6-carboxylate (282 mg), 1 M NaOH solution (1.19 mL), MeOH (5.6 mL), and THF (8.5 mL) was heated at 60° C. for 3 h. After the heating, the mixture was cooled to room temperature and neutralized with 1N HCl. The mixture was extracted with CHCl₃/IPA (4/1), washed with brine and dried over MgSO₄ to give 2-(4-fluorophenyl)-3-[1-(2-methylphenyl)-6-oxo-1,6-dihydropyridazin-3-yl]-4,5,6,7-tetrahydropyrazolo[1,5-a]pyrimidine-6-carboxylic acid (266 mg).

Mass ESI (−) 444 (M−1)

### EXAMPLE 57

A mixture of 2-(4-fluorophenyl)-3-[1-(2-methylphenyl)-6-oxo-1,6-dihydropyridazin-3-yl]-4,5,6,7-tetrahydropyrazolo[1,5-a]pyrimidine-6-carboxylic acid (80.0 mg), WSCD HCl (41.3 mg), and cyclopropylamine (12.3 mg) in CH₂Cl₂ (1 mL) was stirred at rt. After stirring for 2 h, the mixture was extracted with CHCl₃/IPA (5/1), washed with brine, and dried over MgSO₄. After removal of solvent, the crude was purified by column chromatography and crystallized from CHCl₃/IPA (5/1) to give N-cyclopropyl-2-(4-fluorophenyl)-3-[1-(2-methylphenyl)-6-oxo-1,6-dihydropyridazin-3-yl]-4,5,6,7-tetrahydropyrazolo[1,5-a]pyrimidine-6-carboxamide (75 mg).

Mass ESI (+) 507 (M+Na)

¹H-NMR (CDCl₃) δ 0.51 (2H, m), 0.80 (2H, m), 2.20 (3H, s), 2.73 (1H, m), 2.93 (1H, m), 3.54 (2H, m), 4.40 (2H, m), 6.03 (1H, m), 6.30 (1H, m), 6.8-7.58 (8H, m)

### EXAMPLE 58

2-(4-Fluorophenyl)-3-[1-(2-methylphenyl)-6-oxo-1,6-dihydropyridazin-3-yl]-4,5,6,7-tetrahydropyrazolo[1,5-a]pyrimidine-6-carboxamide was obtained according to a similar manner to Example 57.

Mass ESI (+) 467 (M+Na)

¹H-NMR (CDCl₃) δ 2.22 (3H, s), 3.57 (2H, brs), 4.40 (2H, brs), 6.82 (1H, d), 7.01 (1H, d), 7.17 (2H, t), 7.30-7.43 (4H, m), 7.52 (2H, m)

### EXAMPLE 59

2-(4-Fluorophenyl)-N-(2-hydroxyethyl)-3-[1-(2-methylphenyl)-6-oxo-1,6-dihydropyridazin-3-yl]-4,5,6,7-tetrahydropyrazolo[1,5-a]pyrimidine-6-carboxamide was obtained according to a similar manner to Example 57.

Mass ESI (+) 511 (M+Na)

¹H-NMR (CDCl₃) δ 2.21 (3H, s), 2.96 (1H, quin), 3.43 (1H, quin), 3.52 (2H, d), 3.71 (3H, m), 4.35 (2H, t), 6.81 (1H, d), 7.00 (1H, d), 7.15 (2H, t), 7.34 (4H, m), 7.49 (2H, dd)

### EXAMPLE 60

To a mixture of 6-[3-(4-fluorophenyl)-5-({[4-(hydroxymethyl)tetrahydro-2H-thiopyran-4-yl)methyl}amino)-1H-pyrazol-4-yl]-2-(2-methylphenyl)pyridazin-3(2H)-one (650 mg) in CH₃CN (30 mL) was added Et₃N (650 mg) and methanesulfonylchloride (221 mg) at room temperature. The mixture was stirred for 6 h at 100° C. and concentrated. The residue was diluted with 10% aqueous K₂CO₃ and extracted with CHCl₃. The organic layer was washed with brine, dried over MgSO₄, filtered and concentrated in vacuo. The residue was purified by column chromatography on SiO₂ (eluted with Hex/AcOEt=1/4) to give 6-[2-(4-fluorophenyl)-2′,3′,4,5,5′,6′-hexahydrospiro[pyrazolo[1,5-a]pyrimidine-6,4′-thiopyran)-3-yl)-2-(2-methylphenyl)pyridazin-3(2H)-one (320 mg) as a yellow amorphous.

Mass ESI (+) 488(M+1)

¹H-NMR (DMSO-d₆) δ 1.70 (4H, m), 2.08 (3H, s), 2.61 (4H, m), 3.11 (2H, s), 3.89 (2H, s), 6.04 (1H, s), 6.93 (1H, d, J=10 Hz), 7.12 (1H, d, J=10 Hz), 7.22 (2H, t, J=9 Hz), 7.32-7.33 (2H, m), 7.35-7.37 (2H, m), 7.48 (2H, dd, J=9 Hz, 6 Hz)

### EXAMPLE 61

A mixture of 6-[5-({[1-(bromomethyl)cyclohexyl]methyl}amino)-3-(4-fluorophenyl)-1H-pyrazol-4-yl]-2-(2-methylphenyl)pyridazin-3(2H)-one (128 mg) and K₂CO₃ (38.6 mg) in DMF (1.3 mL) was stirred at room temperature for 19 h and then at 40° C. for 3 h. The reaction mixture was poured into H₂O (20 ml), and the products were extracted with AcOEt (20 ml×2). The extract was washed with H₂O (20 ml×3), dried over MgSO₄, filtrated and evaporated. The residue was purified by column chromatography (eluted with CHCl₃). The resultant pale yellow oil was crystallized from AcOEt-hexane to give 6-[2′-(4-fluorophenyl)-4′,5′-dihydrospiro[cyclohexane-1,6′-pyrazolo[1,5-a]pyrimidin]-3′-yl]-2-(2-methylphenyl)pyridazin-3(2H)-one (26.5 mg) as a pale yellow powder.

Mass ESI (+) 470 (M+1)

¹H-NMR (DMSO-d₆) δ 1.32-1.51 (10H, m), 2.08 (3H, s), 3.06 (2H, s), 3.84 (2H, s), 6.00-6.04 (1H, m), 6.93 (1H, d, J=9.8 Hz), 7.12 (1H, d, J=9.8 Hz), 7.18-7.25 (2H, m), 7.30-7.38 (4H, m), 7.45-7.52 (2H, m)

### EXAMPLE 62

6-[2′-(4-Fluorophenyl)-4′,5′-dihydrospiro[cyclopentane-1,6′-pyrazolo[1,5-a]pyrimidin]-3′-yl]-2-(2-methylphenyl)pyridazin-3(2H)-one was obtained according to a similar manner to Example 60.

Mass ESI (+) 456 (M+1)

¹H-NMR (DMSO-d₆) δ 1.38-1.45 (2H, m), 1.51-1.56 (2H, m), 1.64-1.68 (4H, m), 2.09 (3H, s), 3.02 (2H, d, J=2 Hz), 3.85 (2H, s), 6.11 (1H, brs), 6.93 (1H, d, J=10 Hz), 7.01 (1H, d, J=10 Hz), 7.23 (2H, td, J=9.0, 2.0 Hz), 7.34 (4H, m), 7.49 (2H, dd, J=9.0, 5.0 Hz)

### EXAMPLE 63

6-[2′-(4-Fluorophenyl)-2,3,4′,5,5′,6-hexahydrospiro[pyran-4,6′-pyrazolo[1,5-a]pyrimidin)-3′-yl]-2-(2-methylphenyl)pyridazin-3(2H)-one was obtained according to a similar manner to Example 60.

Mass ESI (+) 472 (M+1)

¹H-NMR (DMSO-d₆) δ 1.48 (4H, m), 2.08 (3H, s), 3.15 (2H, s), 3.60 (4H, m), 3.96 (2H, s), 6.07 (1H, s), 6.93 (1H, d, J=10 Hz), 7.12 (1H, d, J=10 Hz), 7.22 (2H, t, J=9 Hz), 7.32-7.37 (4H, m), 7.48 (2H, dd, J=9 Hz, 6 Hz)

### EXAMPLE 64

6-[6-(4-Fluorophenyl)-2,3-dihydro-1H-imidazo[1,2-b]pyrazol-7-yl]-2-(2-methylphenyl)pyridazin-3(2H)-one was obtained according to a similar manner to Example 60.

Mass ESI (−) 386 (M−1)

¹H-NMR (DMSO-d₆) δ 2.21 (3H, s), 4.28-4.61 (2H, m), 6.94 (1H, d), 7.00-7.13 (3H, m), 7.29-7.38 (4H, m), 7.40-7.49 (2H, m)

### EXAMPLE 65

6-[(6S)-6-(Benzyloxy)-2-(4-fluorophenyl)-4,5,6,7-tetrahydropyrazolo[1,5-a]pyrimidin-3-yl]-2-(2-methylphenyl)pyridazin-3(2H)-one was obtained according to a similar manner to Example 60.

Mass ESI (+) 508 (M+1)

¹H-NMR (CDCl₃) δ 2.22 (3H, s), 3.36-3.48 (2H, m), 4.07 (2H, quint, J=4.1 Hz), 4.21 (1H, dd, J=4.1, 12.8 Hz), 4.26 (1H, dd, J=4.1, 12.8 Hz), 4.64 (1H, d, J=12.1 Hz), 4.69 (1H, d, J=12.1 Hz), 5.79 (1H, s), 6.79 (1H, d, J=9.9 Hz), 7.01 (1H, d, J=9.9 Hz), 7.10-7.19 (2H, m), 7.27-7.42 (9H, m), 7.47-7.54 (2H, m).

### EXAMPLE 66

6-[(6R)-6-(Benzyloxy)-2-(4-fluorophenyl)-4,5,6,7-tetrahydropyrazolo[1,5-a]pyrimidin-3-yl)-2-(2-methylphenyl)pyridazin-3(2H)-one was obtained according to a similar manner to Example 60.

Mass ESI (+) 508 (M+1)

¹H-NMR (CDCl₃) δ 2.22 (3H, s), 3.39-3.48 (2H, m), 4.07 (2H, quint, J=4.0 Hz), 4.21 (1H, dd, J=4.0, 12.8 Hz), 4.27 (1H, dd, J=4.0, 12.8 Hz), 4.65 (1H, d, J=12.1 Hz), 4.69 (1H, d, J=12.1 Hz), 5.79 (1H, s), 6.79 (1H, d, J=9.9 Hz), 7.01 (1H, d, J=9.9 Hz), 7.12-7.18 (2H, m), 7.27-7.41 (9H, m), 7.47-7.54 (2H, m)

### EXAMPLE 67

6-[(6S)-6-(Benzyloxy)-2-(2,4-difluorophenyl)-4,5,6,7-tetrahydropyrazolo[1,5-a]pyrimidin-3-yl]-2-(2-methylphenyl)pyridazin-3(2H)-one was obtained according to a similar manner to Example 60.

Mass ESI (+) 526 (M+1)

¹H-NMR (CDCl₃) δ 2.21 (3H, s), 3.39-3.49 (2H, m), 4.04-4.13 (1H, m), 4.20-4.31 (2H, m), 4.65 (1H, d, J=13.1 Hz), 4.69 (1H, d, J=13.1 Hz), 5.87 (1H, s), 6.82 (1H, d, J=10.0 Hz), 6.91-6.98 (1H, m), 6.95 (1H, dd, J=1.6, 10.0 Hz), 6.98-7.07 (1H, m), 7.28-7.42 (9H, m), 7.50-7.59 (1H, m)

### EXAMPLE 68

6-[(6R)-6-(Benzyloxy)-2-(2,4-difluorophenyl)-4,5,6,7-tetrahydropyrazolo[1,5-a]pyrimidin-3-yl]-2-(2-methylphenyl)pyridazin-3(2H)-one was obtained according to a similar manner to Example 60.

Mass ESI (+) 526 (M+1)

¹H-NMR (CDCl₃) δ 2.21 (3H, s), 3.39-3.49 (2H, m), 4.04-4.11 (1H, m), 4.20-4.30 (2H, m), 4.65 (1H, d, J=12.6 Hz), 4.69 (1H, d, J=12.6 Hz), 5.87 (1H, s), 6.83 (1H, d, J=9.9 Hz), 6.91-6.98 (1H, m), 6.95 (1H, dd, J=1.8, 9.9 Hz), 6.98-7.04 (1H, m), 7.27-7.40 (9H, m), 7.55 (1H, dt, J=6.4, 8.2 Hz)

### EXAMPLE 69

6-[(6S)-2-(2,4-Difluorophenyl)-6-(2,2-dimethylpropoxy)-4,5,6,7-tetrahydropyrazolo[1,5-a]pyrimidin-3-yl]-2-(2-methylphenyl)pyridazin-3(2H)-one was obtained according to a similar manner to Example 60.

Mass ESI (+) 506 (M+1)

¹H-NMR (CDCl₃) δ 0.88 (9H, s), 2.21 (3H, s), 3.20 (2H, s), 3.30-3.38 (1H, m), 3.42-3.50 (1H, m), 3.92-3.98 (1H, m), 4.13 (1H, dd, J=5.5, 12.6 Hz), 4.28 (1H, dd, J=4.1, 12.6 Hz), 5.84 (1H, s), 6.83 (1H, d, J=9.9 Hz), 6.90-6.98 (1H, m), 6.96 (1H, dd, J=1.8, 9.9 Hz), 7.01 (1H, dt, J=2.6, 8.2 Hz), 7.32-7.40 (4H, m), 7.54 (1H, dt, J=6.6, 8.2 Hz)

### EXAMPLE 70

Ethyl 2-(2,4-difluorophenyl)-3-[1-(2-methylphenyl)-6-oxo-1,6-dihydropyridazin-3-yl]-4,5,6,7-tetrahydropyrazolo[1,5-a]pyrimidine-6-carboxylate was obtained according to a similar manner to Example 60.

Mass ESI (+) 493 (M+1)

¹H-NMR (CDCl₃) δ 1.27 (3H, t, J=7.0 Hz), 2.20 (3H, s), 3.16-3.21 (1H, m), 3.48-3.52 (1H, m), 3.64-3.68 (1H, m), 4.22 (2H, q, J=6.8 Hz), 4.34 (1H, dd, J=8.1, 12.4 Hz), 4.41 (1H, dd, J=5.3, 12.7 Hz), 5.95 (1H, brs), 6.84 (1H, d, J=9.7 Hz), 6.92-6.97 (m, 2H), 7.00-7.04 (1H, m), 7.32-7.40 (4H, m), 7.52-7.57 (1H, m)

### EXAMPLE 71

Ethyl 2-(4-fluorophenyl)-3-[1-(2-methylphenyl)-6-oxo-1,6-dihydropyridazin-3-yl]-4,5,6,7-tetrahydropyrazolo[1,5-a]pyrimidine-6-carboxylate was obtained according to a similar manner to Example 60.

Mass ESI (+) 474 (M+1)

¹H-NMR (CDCl₃) δ 1.27 (3H, t, J=7.1 Hz), 2.22 (3H, s), 3.15-3.20 (1H, m), 3.47-3.52 (1H, m), 3.65 (1H, ddd, 3.0, 3.0, 12.1 Hz), 4.22 (2H, q, J=7.2 Hz), 4.33 (1H, dd, J=8.2, 12.9 Hz), 4.40 (1H, dd, J=5.5, 12.8 Hz), 5.87 (1H, brs), 6.80 (1H, d, J=10.2 Hz), 7.02 (1H, d J=10.2 Hz), 7.15 (2H, dd, J=8.8, 8.8 Hz), 7.32-7.40 (4H, m), 7.49-7.51 (2H, m)

### EXAMPLE 72

6-[2-(2,4-Difluorophenyl)-5-methyl-4,5,6,7-tetrahydropyrazolo[1,5-a]pyrimidin-3-yl)-2-(2-methylphenyl)pyridazin-3(2H)-one was obtained according to a similar manner to Example 60.

Mass ESI (+) 434 (M+1)

¹H-NMR (CDCl₃) δ 1.19 (3H, d, J=6.6 Hz), 1.87-1.95 (1H, m), 2.11-2.15 (1H, m), 2.23 (3H, s), 3.53-3.57 (1H, m), 4.08-4.14 (1H, m), 4.20-4.25 (1H, m), 5.92 (1H, brs), 6.84 (1H, d, J=10.2 Hz), 6.92-6.96 (2H, m), 7.01 (1H, ddd, J=1.9, 8.1, 8.1 Hz), 7.34-7.37 (4H, m), 1.54 (1H, ddd, 7.2, 8.4, 15.3 Hz)

### EXAMPLE 73

6-[6-(tert-Butoxymethyl)-2-(4-fluorophenyl)-4,5,6,7-tetrahydropyrazolo[1,5-a]pyrimidin-3-yl]-2-(2-methylphenyl)pyridazin-3(2H)-one was obtained according to a similar manner to Example 60.

Mass ESI (+) 488 (M+1)

¹H-NMR (CDCl₃) δ 1.17 (9H, s), 2.23 (3H, s), 2.44 (1H, brs), 3.14-3.20 (1H, m), 3.36-3.46 (3H, m), 3.92 (1H, dd, J=7.9, 12.0 Hz), 4.23 (1H, dd, J=5.1, 12.5 Hz), 5.81 (1H, brs), 6.80 (1H, d, J=9.5 Hz), 7.03 (1H, d, J=10.0 Hz), 7.15 (2H, dd, J=8.2, 8.2 Hz), 7.33-7.40 (4H, m), 7.51 (2H, dd, J=5.1, 8.1 Hz)

### EXAMPLE 74

6-[2′-(2,4-Difluorophenyl)-4′,5′-dihydrospiro[cyclopropane-1,6′-pyrazolo[1,5-a]pyrimidin]-3′-yl]-2-(2-methylphenyl)pyridazin-3(2H)-one was obtained according to a similar manner to Example 60.

Mass ESI (+) 446 (M+1)

¹H-NMR (CDCl₃) δ 0.71-0.76 (4H, m), 2.22 (3H, s), 3.12 (2H, s), 3.95 (2H, s), 5.95 (1H, brs), 6.84 (1H, d, J=10.2 Hz), 6.92-7.04 (3H, m), 7.34-7.39 (4H, m), 7.55 (1H, dd, J=8.2, 14.6 Hz)

### EXAMPLE 75

6-[2′-(4-Fluorophenyl)-4′,5′-dihydrospiro[cyclopropane-1,6′-pyrazolo[1,5-a]pyrimidin)-3′-yl]-2-(2-methylphenyl)pyridazin-3(2H)-one was obtained according to a similar manner to Example 60.

Mass ESI (+) 428 (M+1)

¹H-NMR (CDCl₃) δ 0.71-0.76 (4H, m), 2.23 (3H, s), 3.11 (2H, d, J=1.9 Hz), 3.94 (2H, s), 5.88 (1H, brs), 6.80 (1H, d, J=10.1 Hz), 7.03 (1H, d, J=10.2 Hz), 7.15 (2H, dd, J=8.7, 8.7 Hz), 7.34-7.38 (4H, m), 7.49-7.52 (2H, m)

### EXAMPLE 76

2-(2-Methylphenyl)-6-[2′-(3-methylphenyl)-4′,5′-dihydrospiro[cyclopropane-1,6′-pyrazolo[1,5-a]pyrimidin]-3′-yl]pyridazin-3(2H)-one was obtained according to a similar manner to Example 7.

Mass ESI (+) 424 (M+1).

¹H-NMR (CDCl₃) δ 0.70-0.76 (4H, m), 2.24 (3H, s), 2.40 (3H, s), 3.11 (2H, brs), 3.94 (2H, s), 5.88 (1H, brs), 6.78 (1H, d, J=10.0 Hz), 7.10 (1H, d, J=9.8 Hz), 7.23-7.39 (8H, m)

### EXAMPLE 77

6-[2′-(2,4-Difluorophenyl)-4′,5′-dihydrospiro[cyclobutane-1,6′-pyrazolo[1,5-a]pyrimidin]-3′-yl)-2-(2-methylphenyl)pyridazin-3(2H)-one was obtained according to a similar manner to Example 7.

Mass ESI (+) 941 (2M+Na)

¹H-NMR (CDCl₃) δ 1.96-2.09 (6H, m), 2.22 (3H, s), 3.26 (2H, s), 4.05 (2H, s), 5.90 (1H, brs), 6.82 (1H, d, J=9.7 Hz), 6.92-6.97 (2H, m), 7.01 (1H, ddd, 2.5, 7.8, 7.8 Hz), 7.35-7.39 (4H, m), 7.55 (1H, ddd, J=6.4, 8.3, 8.3 Hz)

### EXAMPLE 78

6-[2′-(4-Fluorophenyl)-4′,5′-dihydrospiro[cyclobutane-1,6′-pyrazolo[1,5-a]pyrimidin]-3′-yl]-2-(2-methylphenyl)pyridazin-3(2H)-one was obtained according to a similar manner to Example 7.

Mass ESI (+) 905 (2M+Na)

¹H-NMR (CDCl₃) δ 1.96-2.11 (6H, m), 2.23 (3H, s), 3.26 (2H, s), 4.04 (2H, s), 5.83 (1H, brs), 6.79 (1H, d, J=10.0 Hz), 7.02 (1H, d, J=9.6 Hz), 7.14 (2H, dd, J=8.8, 8.8 Hz), 7.34-7.39 (4H, m), 7.48-7.51 (2H, m)

### EXAMPLE 79

To a mixture of 6-[3-(2,4-difluorophenyl)-5-({[3-(hydroxymethyl)azetidin-3-yl]methyl}amino)-1H-pyrazol-4-yl]-2-(2-methylphenyl)pyridazin-3(2H)-one (73 mg) and polystyrene-supported triphenylphosphine (1 mmol/d, 280 mg) in dichloromethane (2 ml) was added diethyl azodicarboxylate (44 mL), and the mixture was agitated at room temperature for 1 hour. The insoluble materials were removed by filtration. The filtrate was concentrated under reduced pressure. To the residue were added polystyrene-supported triphenylphosphine (1 mmol/d, 210 mg), dichloromethane (2 ml) and diethyl azodicarboxylate (33 mL), and the mixture was agitated at room temperature overnight. The insoluble materials were removed by filtration. The filtrate was concentrated under reduced pressure. The residue was purified by flash column chromatography on SiO₂ (eluent; 0% methanol in chloroform to 8% methanol in chloroform) to give 6-[2′-(2,4-difluorophenyl)-1-isopropyl-4′,5′-dihydrospiro[azetidine-3,6′-pyrazolo[1,5-a]pyrimidin]-3′-yl]-2-(2-methylphenyl)pyridazin-3(2H)-one as yellow amorphous (54 mg).

Mass EST (+) 503 (M+1)

¹H-NMR (CDCl₃) δ 0.93 (6H, d, J=6.2 Hz), 2.21 (3H, s), 2.36-2.42 (1H, m), 3.07 (2H, d, J=7.8 Hz), 3.23 (2H, d J=7.7 Hz), 3.46 (2H, s), 4.18 (2H, s), 5.96 (1H, brs), 6.83 (1H, d, J=9.9 Hz), 6.92-6.96 (2H, m), 7.01 (1H, ddd, J=2.3, 8.3, 8.3 Hz), 7.32-7.40 (4H, m), 7.54 (1H, dd, 8.3, 14.8 Hz)

### EXAMPLE 80

6-[6-(2,4-Difluorophenyl)-2,2-dimethyl-2,3-dihydro-1H-imidazo[1,2-b]pyrazol-7-yl)-2-(2-methylphenyl)pyridazin-3(2H)-one was obtained according to a similar manner to

### EXAMPLE 7

Mass ESI (+) 434 (M+1)

¹H-NMR (CDCl₃) δ 1.51 (6H, s), 2.21 (3H, s), 4.01 (2H, s), 4.30 (1H, s), 6.86 (1H, d, J=10.1 Hz), 6.91-6.99 (3H, m), 7.33-7.39 (4H, m), 7.56 (1H, dd, J=8.2, 14.7 Hz)

### EXAMPLE 81

6-[(3R)-6-(2,4-Difluorophenyl)-3-methyl-2,3-dihydro-1H-imidazo[1,2-b]pyrazol-7-yl]-2-(2-methylphenyl)pyridazin-3(2H)-one was obtained according to a similar manner to Example 7.

Mass ESI (−) 419 (M+1)

¹H-NMR (CDCl₃) δ 1.60 (3H, d, J=6.6 Hz), 2.20 (3H, s), 3.60-3.64 (1H, m), 4.15 (1H, dd, J=8.2, 8.2 Hz), 4.42 (1H, brs), 4.53-4.60 (1H, m), 6.86 (1H, d, J=9.7 Hz), 6.91-6.98 (2H, m), 7.01 (1H, ddd, J=2.7, 8.3, 8.3 Hz), 7.31-7.38 (4H, m), 7.57 (1H, dd, J=8.3, 15.1 Hz)

### EXAMPLE 82

6-[(3S)-6-(2,4-Difluorophenyl)-3-methyl-2,3-dihydro-1H-imidazo[1,2-b]pyrazol-7-yl]-2-(2-methylphenyl)pyridazin-3(2H)-one was obtained according to a similar manner to Example 7.

Mass ESI (−) 419 (M+1)

¹H-NMR (CDCl₃) δ 1.60 (3H, d, J=6.6 Hz), 2.20 (3H, s), 3.60-3.64 (1H, m), 4.15 (1H, dd, J=8.2, 8.2 Hz), 4.42 (1H, brs), 4.53-4.60 (1H, m), 6.86 (1H, d, J=9.7 Hz), 6.91-6.98 (2H, m), 7.01 (1H, ddd, J=2.7, 8.3, 8.3 Hz), 7.31-7.38 (4H, m), 7.57 (1H, dd, J=8.3, 15.1 Hz)

### EXAMPLE 83

6-[(7aS)-2-(2,4-Difluorophenyl)-6,7,7a,8-tetrahydro-5H-pyrrolo[1′,2′,3,4]imidazo[1,2-b]pyrazol-3-yl]-2-(2-methylphenyl)pyridazin-3(2H)-one was obtained according to a similar manner to Example 7.

Mass ESI (+) 446 (M+1)

¹H-NMR (CDCl₃) δ 1.70-1.78 (1H, m), 1.84-2.00 (2H, m), 2.14 (3H, s), 2.16-2.22 (1H, m), 3.32-3.30 (2H, m), 4.14 (1H, dd, J=5.0, 10.7 Hz), 4.31 (1H, dd, J=9.4, 9.4 Hz), 4.49-4.55 (1H, m), 6.81 (1H, dd, J=9.4, 9.4 Hz), 6.90-6.94 (2H, m), 7.13 (1H, d, J=9.5 Hz), 7.27-7.34 (4H, m), 7.49-7.53 (1H, m)

### EXAMPLE 84

6-[(2R)-6-(4-Fluorophenyl)-2-methyl-2,3-dihydro-1H-imidazo[1,2-b]pyrazol-7-yl]-2-(2-methylphenyl)pyridazin-3(2H)-one was obtained according to a similar manner to Example 60.

Mass ESI (+) 402 (M+1)

¹H-NMR (CDCl₃) δ 1.44 (3H, d, J=6.0 Hz), 2.23 (3H, s), 3.79 (1H, dd, J=8.0 Hz, J=9.5 Hz), 4.37 (1H, dd, J=8.0 Hz, J=9.5 Hz), 4.48 (1H, brs), 4.47-4.55 (1H, m), 6.84 (1H, d, J=10.0 Hz), 7.04 (1H, d, J=10.0 Hz), 7.14 (2H, dd, J=8.5 Hz, J=8.5 Hz), 7.33-7.40 (4H, m), 7.51 (2H, dd, J=5.5 Hz, J=8.5 Hz)

### EXAMPLE 85

6-[6,6-Difluoro-2-(4-fluorophenyl)-4,5,6,7-tetrahydropyrazolo[1,5-a]pyrimidin-3-yl]-2-(2-methylphenyl)pyridazin-3(2H)-one was obtained according to a similar manner to Example 61.

Mass ESI (+) 438 (M+1)

¹H-NMR (CDCl₃) δ 2.22 (3H, s), 3.62 (2H, t, 11.0 Hz), 4.46 (2H, t, 12.0 Hz), 5.99 (1H, brs), 6.83 (1H, d, J=10.1 Hz), 7.01 (1H, d, J=10.1 Hz), 7.16 (2H, dd, J=8.7 Hz, J=8.7 Hz), 7.32-7.42 (4H, m), 7.50 (2H, dd, J=8.7 Hz, J=5.5 Hz)

### EXAMPLE 86

6-[2′-(4-Fluorophenyl)-4′,5′-dihydrospiro[1,3-dioxolane-2,6′-pyrazolo[1,5-a]pyrimidin]-3′-yl]-2-(2-methylphenyl)pyridazin-3(2H)-one was obtained according to a similar manner to Example 61.

Mass ESI (+) 460 (M+1)

¹H-NMR (CDCl₃) δ 2.22 (3H, s), 3.33 (2H, s), 4.06-4.16 (4H, m), 4.15 (2H, s), 5.96 (1H, brs), 6.79 (1H, d, 10.1 Hz), 7.00 (1H, d, J=10.1 Hz), 7.15 (2H, dd, J=8.7 Hz, J=8.7 Hz), 7.31-7.41 (4H, m), 7.50 (2H, dd, J=5.5 Hz, J=8.7 Hz)

### EXAMPLE 87

6-[(2R)-2-[(Benzyloxy)methyl]-6-(4-fluorophenyl)-2,3-dihydro-1H-imidazo[1,2-b]pyrazol-7-yl]-2-(2-methylphenyl)pyridazin-3(2H)-one was obtained according to a similar manner to Example 60.

Mass ESI (+) 530 (M+Na)

¹H-NMR (CD₃Cl) δ 2.95 (3H, s), 3.61 (2H, d, J=6.4 Hz), 3.69 (1H, brs), 4.01-4.06 (2H, m), 4.37 (1H, m), 4.54 (2H, s), 6.86 (1H, d), 7.03 (1H, d, J=9.8 Hz), 7.11-7.66 (13H, m), 8.03 (1H, s)

### EXAMPLE 88

6-[(2S)-2-[(Benzyloxy)methyl]-6-(4-fluorophenyl)-2,3-dihydro-1H-imidazo[1,2-b]pyrazol-7-yl]-2-(2-methylphenyl)pyridazin-3(2H)-one was obtained according to a similar manner to Example 60.

Mass ESI (+) 530 (M+Na)

### EXAMPLE 89

Benzyl 2′-(4-fluorophenyl)-3′-[1-(2-methylphenyl)-6-oxo-1,6-dihydropyridazin-3-yl)-6′,7′-dihydro-1H,4′H-spiro(piperidine-4,5′-pyrazolo[1,5-a]pyrimidine]-1-carboxylate was obtained according to a similar manner to Example 60.

Mass ESI (+) 605 (M+1)

### EXAMPLE 90

6-[2′-(4-Fluorophenyl)-2,3,5,6,6′,7′-hexahydro-4′H-spiro[pyran-4,5′-pyrazolo[1,5-a]pyrimidin]-3′-yl]-2-(2-methylphenyl)pyridazin-3(2H)-one was obtained according to a similar manner to Example 60.

Mass ESI (+) 472 (M+1)

¹H-NMR (CDCl₃) δ 1.48-1.56 (2H, m), 1.68-1.79 (2H, m), 2.01 (2H, t, J=6 Hz), 2.25 (3H, s), 3.07-3.22 (2H, m), 3.60-3.75 (2H, m), 4.18 (2H, t, J=6 Hz), 6.79-6.83 (1H, m), 6.83 (1H, d, J=10 Hz), 7.06 (1H, d, J=10 Hz), 7.14-7.19 (2H, m), 7.35-7.39 (4H, m), 7.48-7.53 (2H, m)

### EXAMPLE 91

6-[2-(4-Fluorophenyl)-1′,1′-dioxido-2′,3′,4,5,5′, 6′-hexahydrospiro[pyrazolo[1,5-a]pyrimidine-6,4′-thiopyran]-3-yl]-2-(2-methylphenyl)pyridazin-3(2H)-one was obtained according to a similar manner to Example 60.

Mass ESI (+) 542 (M+Na)

¹H-NMR (DMSO-d₆) δ 1.86-1.98 (4H, m), 2.09 (3H, s), 3.13 (4H, m), 3.22 (2H, brs), 4.02 (2H, s), 6.11 (1H, br), 6.94 (1H, d, J=10 Hz), 7.13 (1H, d, J=10 Hz), 7.23 (2H, t, J=9 Hz), 7.32-7.38 (4H, m), 7.49 (2H, dd, J=9 Hz, 5 Hz)

### EXAMPLE 92

tert-Butyl 2′-(4-fluorophenyl)-3′-[1-(2-methylphenyl)-6-oxo-1,6-dihydropyridazin-3-yl]-4′,5′-dihydro-1H-spiro[piperidine-4,6′-pyrazolo[1,5-a]pyrimidine]-1-carboxylate was obtained according to a similar manner to Example 60.

Mass ESI (+) 593 (M+Na)

¹H-NMR (CDCl₃) δ 1.39-1.51 (4H, m), 1.46, 1.47 (9H, s), 2.23, 2.93 (3H, s), 3.12-3.32 (8H, m), 5.88, 6.20 (1H, br), 6.57-7.56 (10H, m)

### EXAMPLE 93

6-[(5S)-5-({[tert-Butyl(diphenyl)silyl]oxy}methyl)-2-(4-fluorophenyl)-4,5,6,7-tetrahydropyrazolo[1,5-a]pyrimidin-3-yl]-2-(2-methylphenyl)pyridazin-3(2H)-one was obtained according to a similar manner to Example 60.

¹H-NMR (DMSO-d₆) δ 0.90 (9H, s), 1.97-2.00 (1H, m), 2.03 (3H, s), 2.15-2.23 (1H, m), 3.52-3.68 (3H, m), 4.00-4.05 (2H, m), 6.03 (1H, brs), 6.92 (1H, d, J=10 Hz), 7.07 (1H, d, J=10 Hz), 7.19-7.57 (18H, m)

### EXAMPLE 94

6-[(5R)-5-({[tert-Butyl(diphenyl)silyl]oxy}methyl)-2-(4-fluorophenyl)-4,5,6,7-tetrahydropyrazolo[1,5-a]pyrimidin-3-yl]-2-(2-methylphenyl)pyridazin-3(2H)-one was obtained according to a similar manner to Example 60.

¹H-NMR (DMSO-d₆) δ 0.90 (9H, s), 1.97-2.00 (1H, m), 2.03 (3H, s), 2.15-2.23 (1H, m), 3.52-3.68 (3H, m), 4.00-4.05 (2H, m), 6.03 (1H, brs), 6.92 (1H, d, J=10 Hz), 7.07 (1H, d, J=10 Hz), 7.19-7.57 (18H, m)

### EXAMPLE 95

A mixture of 6-[(6S)-6-(benzyloxy)-2-(4-fluorophenyl)-4,5,6,7-tetrahydropyrazolo[1,5-a]pyrimidin-3-yl]-2-(2-methylphenyl)pyridazin-3(2H)-one (430 mg) and palladium hydroxide (250 mg, 20% wt. on carbon) in EtOH (20 mL) was stirred under a hydrogen atmosphere at 45-50° C. for 6 hours. To the reaction mixture was further added palladium hydroxide (50 mg, 20% wt. on carbon) and the mixture was stirred under a hydrogen atmosphere at 50° C. for 1 hour. After the catalyst was filtered off, the filtrate was concentrated in vacuo. The residue was purified by flash silica-gel column chromatography (gradient elution: AcOEt/hexane=1/2 to 1/1) followed by crystallization from a mixed solvent of diethyl ether and dichloromethane to give 6-[(6S)-2-(4-fluorophenyl)-6-hydroxy-4,5,6,7-tetrahydropyrazolo[1,5-a]pyrimidin-3-yl]-2-(2-methylphenyl)pyridazin-3(2H)-one (115 mg) as pale yellow crystals.

Mass ESI (+) 418 (M+1)

¹H-NMR (CDCl₃) δ 2.23 (3H, s), 2.37 (1H, d, J=7.3 Hz), 3.35-3.45 (2H, m), 4.17-4.23 (1H, m), 4.26 (1H, dd, J=3.4 Hz, 13.1 Hz), 4.38-4.46 (1H, m), 5.85 (1H, s), 6.81 (1H, d, J=9.6 Hz), 7.03 (1H, d, J=9.6 Hz), 7.12-7.19 (2H, m), 7.31-7.42 (4H, m), 7.48-7.54 (2H, m)

### EXAMPLE 96

6-{6-[(Methylamino)methyl]-2-(3-methylphenyl)-4,5,6,7-tetrahydropyrazolo[1,5-a]pyrimidin-3-yl}-2-(2-methylphenyl)pyridazin-3(2H)-one was obtained according to a similar manner to Example 95.

Mass ESI (+) 441 (M+1)

¹H-NMR (CDCl₃) δ 2.23 (3H, s), 2.37-2.40 (1H, m), 2.45 (3H, s), 2.69 (2H, d, J=6.8 Hz), 3.14 (1H, dd, J=9.1 Hz, J=9.1 Hz), 3.47 (1H, d, J=12.0 Hz), 3.86 (1H, dd, J=8.2 Hz, 12.9 Hz), 4.27 (1H, dd, J=5.1, 12.9 Hz), 5.83 (1H, brs), 6.77 (1H, d, J=10.2 Hz), 7.08 (1H, d, J=9.9 Hz), 7.23-7.39 (8H, m)

### EXAMPLE 97

6-[6-(Aminomethyl)-2-(2,4-difluorophenyl)-4,5,6,7-tetrahydropyrazolo[1,5-a]pyrimidin-3-yl]-2-(2-methylphenyl)pyridazin-3(2H)-one was obtained according to a similar manner to Example 95.

Mass ESI (+) 449 (M+1)

¹H-NMR (DMSO-d₆) δ 2.03 (3H, s), 2.90-2.92 (2H, m), 3.12 (1H, dd, J=8.3, 12.4 Hz), 3.95 (1H, dd, J=7.8, 12.9 Hz), 4.28 (1H, dd, J=5.2, 12.5 Hz), 6.18 (1H, brs), 6.96 (1H, d, J=10.3 Hz), 7.13-7.18 (2H, m), 7.22 (1H, d, J=7.4 Hz), 7.28-7.37 (4H, m), 7.51-7.56 (1H, m)

### EXAMPLE 98

6-{6-[(tert-Butylamino)methyl]-2-(2,4-difluorophenyl)-4,5,6,7-tetrahydropyrazolo[1,5-a]pyrimidin-3-yl}-2-(2-methylphenyl)pyridazin-3(2H)-one was obtained according to a similar manner to Example 95.

Mass ESI (+) 505 (M+1)

¹H-NMR (CDCl₃) δ 1.14 (9H, d, J=5.1 Hz), 2.18 (3H, s), 2.21 (2H, s), 2.52 (1H, brs), 3.27-3.53 (1H, m), 3.52-3.57 (1H, m), 3.91-4.02 (1H, m), 4.29-4.34 (1H, m), 5.86 (1H, brs), 6.84 (1H, d, J=10.1 Hz), 6.90-7.01 (4H, m), 7.29-7.36 (3H, m), 7.49-7.54 (1H, m)

### EXAMPLE 99

6-[(6S)-2-(2,4-Difluorophenyl)-6-hydroxy-4,5,6,7-tetrahydropyrazolo[1,5-a]pyrimidin-3-yl]-2-(2-methylphenyl)pyridazin-3(2H)-one was obtained according to a similar manner to Example 95.

Mass ESI (+) 436 (M+1)

¹H-NMR (CDCl₃) δ 2.21 (3H, s), 2.37 (1H, d, J=6.9 Hz), 3.35-3.49 (2H, m), 4.18-4.25 (1H, m), 4.27 (1H, dd, J=3.4, 13.1 Hz), 4.40-4.47 (1H, m), 5.92 (1H, s), 6.84 (1H, d, J=9.7 Hz), 6.92-7.06 (2H, m), 6.97 (1H, dd, J=2.1, 9.7 Hz), 7.30-7.42 (4H, m), 7.55 (1H, dt, J=6.4, 8.4 Hz)

### EXAMPLE 100

6-[(6R)-2-(4-Fluorophenyl)-6-hydroxy-4,5,6,7-tetrahydropyrazolo[1,5-a]pyrimidin-3-yl]-2-(2-methylphenyl)pyridazin-3(2H)-one was obtained according to a similar manner to Example 95.

Mass ESI (+) 418 (M+1)

¹H-NMR (CDCl₃) δ 2.22 (3H, s), 3.36-3.50 (2H, m), 4.20-4.53 (3H, m), 6.00 (1H, s), 6.83 (1H, d, J=9.6 Hz), 7.02 (1H, d, J=9.6 Hz), 7.18 (2H, t, J=8.2 Hz), 7.30-7.41 (4H, m), 7.50-7.58 (2H, m)

### EXAMPLE 101

6-[(6R)-2-(2,4-Difluorophenyl)-6-hydroxy-4,5,6,7-tetrahydropyrazolo[1,5-a]pyrimidin-3-yl]-2-(2-methylphenyl)pyridazin-3(2H)-one was obtained according to a similar manner to Example 95.

Mass ESI (+) 436 (M+1)

¹H-NMR (CDCl₃) δ 2.21 (3H, s), 2.33 (1H, d, J=6.9 Hz), 3.35-3.49 (2H, m), 4.18-4.25 (1H, m), 4.27 (1H, dd, J=3.4, 13.1 Hz), 4.40-4.47 (1H, m), 5.92 (1H, s), 6.85 (1H, d, J=10.0 Hz), 6.92-7.06 (2H, m), 6.97 (1H, dd, J=2.1, 10.0 Hz), 7.30-7.41 (4H, m), 7.55 (1H, dt, J=6.4, 8.2 Hz)

### EXAMPLE 102

6-[2′-(4-Fluorophenyl)-6′,7′-dihydro-4′H-spiro[piperidine-4,5′-pyrazolo[1,5-a]pyrimidin]-3′-yl]-2-(2-methylphenyl)pyridazin-3(2H)-one was obtained according to a similar manner to Example 95.

Mass ESI (+) 471 (M+1)

¹H-NMR (DMSO-d₆) δ 1.38-1.82 (6H, m), 2.10 (3H, s), 2.71-2.90 (2H, m), 2.96-3.11 (2H, m), 4.01-4.17 (2H, m), 6.23 (1H, s), 6.97 (1H, d, J=10.2 Hz), 7.15 (1H, d, J=9.4 Hz), 7.21-7.29 (2H, m), 7.32-7.44 (4H, m), 7.45-7.55 (2H, m)

### EXAMPLE 103

6-[(2R)-6-(4-Fluorophenyl)-2-(hydroxymethyl)-2,3-dihydro-1H-imidazol-1,2-b]pyrazol-7-yl]-2-(2-methylphenyl)pyridazin-3(2H)-one was obtained according to a similar manner to Example 95.

Mass ESI (+) 440 (M+Na)

¹H-NMR (CDCl₃) δ 2.22 (3H, s), 3.65-3.86 (2H, m), 4.05-4.19 (2H, m), 4.36 (1H, t), 4.54 (1H, m), 6.85 (1H, d), 7.03 (1H, d), 7.09-7.55 (8H, m)

### EXAMPLE 104

6-[(2S)-6-(4-Fluorophenyl)-2-(hydroxymethyl)-2,3-dihydro-1H-imidazo[1,2-b]pyrazol-7-yl]-2-(2-methylphenyl)pyridazin-3(2H)-one was obtained according to a similar manner to Example 95.

Mass ESI (+) 440 (M+Na)

### EXAMPLE 105

To a mixture of tert-butyl 2′-(4-fluorophenyl)-3′-[1-(2-methylphenyl)-6-oxo-1,6-dihydropyridazin-3-yl]-4′,5′-dihydro-1H-spiro(piperidine-4,6′-pyrazolo[1,5-a]pyrimidine]-1-carboxylate (223 mg) and dioxane (2.23 mL) was added 4 M HCl-dioxane (2.23 mL), and the whole mixture was stirred at rt for 4 h. The reaction mixture was evaporated, and the residue was added H₂O (20 ml), and the mixture was washed with CHCl₃ (20 ml×2). The aqueous layer was neutralized with NaHCO₃ and extracted with CHCl₃ (20 ml×2). The extract was dried over anh. MgSO₄, filtrated and evaporated. To the residue was added AcOEt and 4 M HCl-dioxane, and the mixture was then evaporated. The residue was dried in vacuo to give 6-[2′-(4-fluorophenyl)-4′,5′-dihydrospiro[piperidine-4,6′-pyrazolo[1,5-a]pyrimidin]-3′-yl]-2-(2-methylphenyl)pyridazin-3(2H)-one hydrochloride (156 mg) as a pale yellow foam.

Mass ESI (+) 471 (M+1)

### EXAMPLE 106

To a mixture of 6-[2′-(4-fluorophenyl)-4′,5′-dihydrospiro[piperidine-4,6′-pyrazolo[1,5-a]pyrimidin]-3′-yl]-2-(2-methylphenyl)pyridazin-3(2H)-one hydrochloride (150 mg) and MeCN (3 mL) were added ethyl iodide (215 mg) and K₂CO— (123 mg), and the whole mixture was stirred at rt for 19 h. The reaction mixture was evaporated, and the residue was diluted with H₂O (20 ml) and extracted with CHCl₃ (20 ml×2). The extract was dried over anhydrous MgSO₄, filtrated and evaporated. The residue was purified by column chromatography (eluent: 5% MeOH in CHCl₃). The obtained compound (51 mg) was dissolved in dioxane (0.5 mL), treated with 4 M HCl-dioxane (0.1 mL) and concentrated to give 6-[1-ethyl-2′-(4-fluorophenyl)-4′,51-dihydrospiro[piperidine-4,6′-pyrazolo[1,5-a]pyrimidin]-3′-yl]-2-(2-methylphenyl)pyridazin-3(2H)-one hydrochloride (51.6 mg) as a pale yellow foam.

Mass ESI (+) 499 (M+1)

¹H-NMR (DMSO-d₆) δ 1.20-1.28 (3H, m), 1.68-1.88 (4H, m), 2.06-2.13 (3H, m), 2.96-3.16 (5H, m), 3.29-3.43 (3H, m), 3.83-3.86 (1H, m), 4.17-4.23 (1H, m), 6.95 (1H, d, J=9.8 Hz), 7.11-7.17 (1H, m), 7.20-7.28 (2H, m), 7.30-7.39 (4H, m), 7.46-7.54 (2H, m), 10.04-10.28 (1H, m)

### EXAMPLE 107

To a mixture of 6-[1-acetyl-2′-(4-fluorophenyl)-4′,5′-dihydrospiro[piperidine-4,6′-pyrazolo[1,5-a]pyrimidin]-3′-yl]-2-(2-methylphenyl)pyridazin-3(2H)-one (120 mg) and CH₂Cl₂ (2 mL) was added Ac₂O (0.034 mL) and triethylamine (0.086 mL) on ice bath, and the whole was stirred at room temperature for 4 h. The reaction mixture was diluted with AcOEt (30 ml), washed with H₂O and brine (20 ml, each), dried over MgSO₄ and evaporated. The residue was purified by column chromatography (eluent: 5% MeOH in CHCl₃) to give 6-[1-acetyl-2′-(4-fluorophenyl)-4′,5′-dihydrospiro[piperidine-4,6′-pyrazolo[1,5-a]pyrimidin]-3′-yl]-2-(2-methylphenyl)pyridazin-3(2H)-one (74 mg).

Mass ESI (+) 535 (M+Na)

¹H-NMR (CDCl₃) δ 1.56-1.68 (4H, m), 2.10 (3H, s), 2.23 (3H, S), 3.17-3.23 (2H, m), 3.46-3.59 (3H, m), 3.71-3.79 (1H, m), 4.02-4.16 (2H, m), 5.96 (1H, br s), 6.83 (1H, d, J=9.8 Hz), 7.04 (1H, d, J=9.8 Hz), 7.18 (2H, t, J=8.7 Hz), 7.31-7.43 (4H, m), 7.51-7.57 (2H, m)

### EXAMPLE 108

6-[2′-(4-Fluorophenyl)-1-(2-hydroxyethyl)-4′,5′-dihydrospiro[piperidine-4,6′-pyrazolo[1,5-a]pyrimidin]-3′-yl]-2-(2-methylphenyl)pyridazin-3(2H)-one was obtained according to a similar manner to Example 106.

Mass ESI (+) 515 (M+1)

¹H-NMR (CDCl₃) δ 1.68-1.73 (4H, m), 2.23 (3H, s), 2.53-2.67 (6H, m), 3.14-3.18 (2H, m), 3.66 (2H, t, J=5 Hz), 3.96 (2H, s), 5.81 (1H, br), 6.80 (1H, d, J=10 Hz), 7.03 (1H, d, J=10 Hz), 7.12-7.18 (2H, m), 7.34-7.40 (4H, m), 7.48-7.52 (2H,

### EXAMPLE 109

6-[2′-(4-Fluorophenyl)-1-(3-hydroxypropyl)-4′,5′-dihydrospiro[piperidine-4,61-pyrazolo[1,5-a]pyrimidin]-3′-yl]-2-(2-methylphenyl)pyridazin-3(2H)-one was obtained according to a similar manner to Example 106.

Mass ESI (+) 529 (M+1)

¹H-NMR (CDCl₃) δ 1.57-1.80 (6H, m), 2.22 (3H, s), 2.42-2.79 (6H, m), 3.14 (2H, s), 3.80 (2H, t, J=5 Hz), 3.95 (2H, s), 5.80 (1H, s), 6.80 (1H, d, J=10 Hz), 7.03 (1H, d, J=10 Hz), 7.10-7.20 (2H, m), 7.32-7.42 (4H, m), 7.47-7.53 (2H, m)

### EXAMPLE 110

6-[1-Acetyl-2′-(4-fluorophenyl)-6′,7′-dihydro-4′H-spiro[piperidine-4,5′-pyrazolo[1,5-a]pyrimidin]-3′-yl]-2-(2-methylphenyl)pyridazin-3(2H)-one was obtained according to a similar manner to Example 107.

Mass ESI (+) 513 (M+1)

¹H-NMR (DMSO-d₆) δ 1.44-1.70 (4H, m), 1.95-2.02 (5H, m), 2.08 (3H, s), 2.78 (1H, t), 3.52 (1H, d), 3.89 (1H, d), 4.08 (2H, t), 6.50 (1H, brs), 6.95 (1H, d), 7.08 (1H, d), 7.22-7.41 (6H, m), 7.46-7.58 (2H, q)

### EXAMPLE 111

N-({2-(2,4-Difluorophenyl)-3-[1-(2-methylphenyl)-6-oxo-1,6-dihydropyridazin-3-yl]-4,5,6,7-tetrahydropyrazolo[1,5-a]pyrimidin-6-yl}methyl)cyclopropanecarboxamide was obtained according to a similar-manner to Example 107.

Mass ESI (+) 517 (M+1)

¹H-NMR (CDCl₃)δ 0.72-0.76 (2H, m), 0.93-0.96 (2H, m), 1.29-1.34 (1H, m), 2.20 (3H, s), 2.52 (1H, brs), 3.14 (1H, dd, J=8.7, 8.7 Hz), 3.27-3.32 (1H, m), 3.35-3.42 (2H, m), 3.91 (1H, dd, J=7.2, 12.4 Hz), 4.21 (1H, dd, J=4.7, 12.5 Hz), 5.87 (1H, brs), 6.08 (1H, brs), 6.83 (1H, d, J=10.1 Hz), 6.92-6.97 (2H, m), 7.00-7.05 (1H, m), 7.31-7.36 (4H, m), 7.53 (1H, dd, J=8.2, 15.1 Hz)

### EXAMPLE 112

1-({2-(2,4-Difluorophenyl)-3-[1-(2-methylphenyl)-6-oxo-1,6-dihydropyridazin-3-yl]-4,5,6,7-tetrahydropyrazolo[1,5-a]pyrimidin-6-yl}methyl)-3-ethylurea was obtained according to a similar manner to Example 107.

Mass ESI (+) 520 (M+1)

¹H-NMR (CDCl₃) δ 1.07 (3H, t, J=7.4 Hz), 2.19 (3H, s), 2.48 (1H, brs), 3.12-3.22 (4H, m), 3.26 (1H, brs), 3.40 (1H, d, J=10.9 HZ), 3.90 (1H, dd, J=6.9, 12.3 Hz), 4.19 (1H, dd, J=5.0, 12.9 Hz), 4.54 (1H, brs), 4.86 (1H, brs), 5.86 (1H, brs), 6.82 (1H, d, J=10.0 Hz), 6.92-6.97 2H, m), 7.01 (1H, ddd, J=2.4, 7.9, 7.9 Hz), 7.30-7.36 (4H, m), 7.52 (1H, dd, J=8.2, 14.5 Hz)

### EXAMPLE 113

To a mixture of 6-[2-(4-fluorophenyl)-6-(hydroxymethyl)-4,5,6,7-tetrahydropyrazolo[1,5-a]pyrimidin-3-yl]-2-(2-methylphenyl)pyridazin-3(2H)-one (300 mg) in pyridine (1.47 g) was added acetic anhydride (277 mg) at room temperature. After stirring for 14 h, the mixture was concentrated and partitioned between EtOAc and 5% aqueous citric acid. The organic layer was washed with saturated aqueous NaHCO₃ and brine, dried over Na₂SO₄, filtered and concentrated. The residue was triturated with IPE to give {2-(4-fluorophenyl)-3-[1-(2-methylphenyl)-6-oxo-1,6-dihydropyridazin-3-yl]-4,5,6,7-tetrahydropyrazolo[1,5-a]pyrimidin-6-yl}methyl acetate (248 mg) as yellow powder.

Mass ESI (+) 474 (M+1)

¹H-NMR (DMSO-d₆) δ 2.05 (3H, s), 2.09 (3H, s), 3.04-3.14 (1H, m), 3.28-3.41 (2H, m), 3.84-3.92 (1H, m), 4.06 (2H, d, J=7.3 Hz), 4.18 (1H, dd, J=4.8, 11.7 Hz), 6.04 (1H, s), 6.93 (1H, d, J=10.4 Hz), 7.09 (1H, d, J=10.4 Hz), 7.19-7.26 (2H, m), 7.31-7.38 (4H, m), 7.45-7.51 (2H, m).

### EXAMPLE 114

tert-Butyl 2-(4-fluorophenyl)-3-[1-(2-methylphenyl)-6-oxo-1,6-dihydropyridazin-3-yl]-4,5,6,7-tetrahydropyrazolo[1,5-a]pyrimidin-6-yl carbonate was obtained according to a similar manner to Example 113.

¹H-NMR (CDCl₃) δ 1.48 (9H, s), 2.22 (3H, s), 3.45-3.61 (2H, m), 4.28-4.41 (2H, m), 5.18-5.26 (1H, m), 5.83 (1H, s), 6.80 (1H, d, J=9.9 Hz), 7.01 (1H, d, J=9.9 Hz), 7.15 (2H, t, J=9.2 Hz), 7.30-7.43 (4H, m), 7.47-7.55 (2H, m)

### EXAMPLE 115

To a solution of 6-[2-(4-fluorophenyl)-6-(hydroxymethyl)-4,5,6,7-tetrahydropyrazolo[1,5-a]pyrimidin-3-yl]-2-(2-methylphenyl)pyridazin-3(2H)-one (1.08 g) in DMSO (15 mL) was added Et3N (2.53 g), and the solution was stirred at room temperature for 5 minutes. To the solution was added dropwise SO₃-pyridine complex (1.59 g) in DMSO (5 mL) over 15 minutes period, and the solution was stirred at room temperature for 5 hours. To the solution was added AcOEt (30 mL), and the solution was washed successively with 10% aqueous citric acid solution (30 mL×4), saturated aqueous NaHCO₃ solution (30 mL) and brine (30 mL) The organic layer was dried over anhydrous MgSO₄, and filtered off. The filtrate was concentrated in vacuo to give 2-(4-fluorophenyl)-3-(1-(2-methylphenyl)-6-oxo-1,6-dihydropyridazin-3-yl]-4,5,6,7-tetrahydropyrazolo[1,5-a]pyrimidine-6-carbaldehyde (863 mg) as a yellow solid.

Mass ESI (−) 428 (M−1)

¹H NMR (CDCl₃) δ 2.22 (3H, s), 3.09 (1H, m), 3.63-3.76 (2H, s), 4.31 (1H, dd, J=5.0 Hz, J=13.0 Hz), 4.60 (1H, dd, J=5.0 Hz, J=13.0 Hz), 5.87 (1H, brs), 6.80 (1H, d, J=10.1 Hz), 7.00 (1H, d, J=10.1 Hz), 7.15 (2H, dd, J=8.7 Hz, J=8.7 Hz), 7.31-7.41 (4H, m), 7.49 (2H, dd, J=8.7 Hz, J=5.5 Hz), 9.79 (1H, s)

### EXAMPLE 116

To a solution of 2-(4-fluorophenyl)-3-[1-(2-methylphenyl)-6-oxo-1,6-dihydropyridazin-3-yl]-4,5,6,7-tetrahydropyrazolo[1,5-a]pyrimidine-6-carbaldehyde (215 mg) in MeOH (7 mL) were added 50% aqueous hydroxylamine solution (1.5 mL) and dichloromethane (1 mL) at room temperature. After stirring for 1 day at room temperature, the mixture was evaporated in vacuo. The crystalline residue was washed with a mixed solvent of dichloromethane and diethyl ether to give 2-(4-fluorophenyl)-3-[1-(2-methylphenyl)-6-oxo-1,6-dihydropyridazin-3-yl]-4,5,6,7-tetrahydropyrazolo[1,5-a]pyrimidine-6-carbaldehyde oxime (150 mg) as a pale yellow solid.

Mass ESI (+) 445 (M+1)

¹H-NMR (CDCl₃) δ 2.22 (3H, s), 3.08-3.17 (1H, m), 3.23-3.31 (1H, m), 3.53-3.62 (1H, m), 4.24 (2H, dd, J=8.9, 13.1 Hz), 4.49 (2H, dd, J=4.8, 13.1 Hz), 5.91 (1H, s), 6.81 (1H, d, J=10.1 Hz), 7.02 (1H, d, J=10.1 Hz), 7.13-7.19 (2H, m), 7.31-7.41 (4H, m), 7.46-7.53 (3H, m), 8.95 (1H, s)

### EXAMPLE 117

A solution of 2-(4-fluorophenyl)-3-[1-(2-methylphenyl)-6-oxo-1,6-dihydropyridazin-3-yl]-4,5,6,7-tetrahydropyrazolo[1,5-a]pyrimidine-6-carbaldehyde oxime (45 mg) in formic acid (1 mL) was stirred under reflux for 1 day. To the reaction mixture was added water (20 mL) and the mixture was extracted with dichloromethane. The organic layer was washed successively with 5% aqueous Na₂CO₃ solution and brine, dried over Na₂SO₄ and concentrated in vacuo. The residue was purified by flash silica-gel column chromatography (gradient elution: MeOH/chloroform=0/1 to 1/19) followed by crystallization from a mixed solvent of diethyl ether and dichloromethane to give 2-(4-fluorophenyl)-3-[1-(2-methylphenyl)-6-oxo-1,6-dihydropyridazin-3-yl]-4,5,6,7-tetrahydropyrazolo[1,5-a]pyrimidine-6-carbonitrile (20 mg) as a pale yellow solid.

Mass ESI (+) 427 (M+1)

¹H-NMR (CDCl₃) δ 2.22 (3H, s), 3.39 (1H, quint, J=5.2 Hz), 4.59-4.67 (2H, m), 4.41 (2H, d, J=5.2 Hz), 6.01 (1H, s), 6.82 (1H, d, J=9.9 Hz), 7.01 (1H, d, J=9.9 Hz), 7.13-7.20 (2H, m), 7.29-7.43 (4H, m), 7.46-7.53 (2H, m)

### EXAMPLE 118

A mixture of 6-[2-(4-fluorophenyl)-6-(iodomethyl)-4,5,6,7-tetrahydropyrazolo[1,5-a]pyrimidin-3-yl]-2-(2-methylphenyl)pyridazin-3(2H)-one (1.43 g) and NaOMe (612 mg) in MeOH (14.3 mL) was refluxed for 12 h. After removal of solvent, the mixture was extracted with EtOAc, washed with 5% citric acid, and dried over MgSO₄. After removal of solvent, 6-[2-(4-fluorophenyl)-6-methylene-4,5,6,7 tetrahydropyrazolo[1,5-a]pyrimidin-3-yl)-2-(2-methylphenyl)pyridazin-3(2H)-one (1.0 g) was obtained as a yellow amorphous solid.

Mass ESI (+) 414 (M+1)

¹H-NMR (CDCl₃) δ 2.22 (3H, s), 3.90 (2H, s), 4.80 (2H, s), 5.28 (1H, s), 5.33 (1H, s), 5.97 (1H, s), 6.82 (1H, d, J=10.0 Hz), 7.02 (1H, d, J=10.4 Hz), 7.14-7.20 (2H, m), 7.31-7.40 (4H, m), 7.50-7.56 (2H, m).

### EXAMPLE 119

A mixture of 6-[2-(4-fluorophenyl)-6-methylene-4,5,6,7-tetrahydropyrazolo[1,5-a]pyrimidin-3-yl)-2-(2-methylphenyl)pyridazin-3(2H)-one (100 mg), 10% palladium on carbon (26 mg), and MeOH (1 mL) was stirred for 5 h under H₂ gas until the reaction was complete. The mixture was filtrated through the Celite® pad and the filtrate was evaporated. The crude was purified by column chromatography (eluent: 1% MeOH in CHCl₃) to give 6-[2-(4-fluorophenyl)-6-methyl-4,5,6,7-tetrahydropyrazolo[1,5-a]pyrimidin-3-yl]-2-(2-methylphenyl)pyridazin-3(2H)-one (43.8 mg).

Mass ESI (+) 438 (M+Na)

¹H-NMR (CDCl₃) δ 1.02 (3H, d), 2.08 (3H, s), 2.21 (1H, brs), 2.88 (1H, t), 3.27 (1H, m), 3.63 (1H, dd), 4.12 (1H, dd), 6.92 (1H, d), 7.08 (1H, d), 7.23 (2H, t), 7.34 (4H, m), 7.47 (2H, dd).

### EXAMPLE 120

A mixture of 6-[2-(4-fluorophenyl)-6-methylene-4,5,6,7-tetrahydropyrazolo[1,5-a]pyrimidin-3-yl]-2-(2-methylphenyl)pyridazin-3(2H)-one (150 mg), OsO₄ (46 mg), N-methylmorpholine N-oxide (55.3 mg), H₂O (0.6 mL), acetone (0.6 mL), and MeCN (0.6 mL) was stirred for 3 weeks and then filtered through the Celite® pad. The filtrate was evaporated and the residue was purified by column chromatography to give 6-[2-(4-fluorophenyl)-6-(hydroxymethyl)pyrazolo[1,5-a]pyrimidin-3-yl]-2-(2-methylphenyl)pyridazin-3(2H)-one (37.0 mg)

Mass ESI (+) 450 (M+Na)

¹H-NMR (CDCl₃) δ 2.14 (3H, s), 4.83 (2H, s), 7.04 (2H, t), 7.14 (3H, m), 7.30 (2H, m), 7.70 (2H, dd), 7.92 (1H, d), 8.58 (1H, s), 8.71 (1H, s)

### EXAMPLE 121

A mixture of 6-[2′-(4-fluorophenyl)-4′,5′-dihydrospiro[1,3-dioxolane-2,6′-pyrazolo[1,5-a]pyrimidin]-3′-yl]-2-(2-methylphenyl)pyridazin-3(2H)-one (500 mg) and conc. aqueous HCl solution (10 ml) was stirred at 80° C. for overnight. To the solution were added water (40 mL) and AcOEt (60 mL), and the biphasic solution was basified with Na₂CO₃. The aqueous layer was removed, and the organic layer was washed successively with saturated aqueous NaHCO₃ solution (30 mL×2) and brine (20 mL). The organic layer was dried over anhydrous MgSO₄, and filtered off. The filtrate was concentrated in vacuo to give 2-(4-fluorophenyl)-3-[1-(2-methylphenyl)-6-oxo-1,6-dihydropyridazin-3-yl]-4,5-dihydropyrazolo[1,5-a]pyrimidin-6(7H)-one (453 mg) as a yellow oil.

Mass ESI (−) 414 (M−1)

¹H-NMR (CDCl₃) δ 2.21 (3H, s), 3.97 (1H, s), 4.74 (1H, s), 6.08 (1H, brs), 6.86 (1H, d, J=9.6 Hz), 7.06 (1H, d, J=9.6 Hz), 7.18 (2H, dd, J=8.7 Hz, J=8.7 Hz), 7.32-7.42 (4H, m), 7.51 (2H, dd, J=8.7 Hz, J=5.5 Hz)

### EXAMPLE 122

To the solution of 2-(4-fluorophenyl)-3-[1-(2-methylphenyl)-6-oxo-1,6-dihydropyridazin-3-yl]-4,5-dihydropyrazolo[1,5-a]pyrimidin-6(7H)-one (453 mg) in EtOH (5 mL) was added a solution of hydroxylamine hydrochloride (94.7 mg) in water (0.35 mL), and the solution was stirred at room temperature for 1.5 hours. To the solution was added CHCl₃ (50 mL), and the suspension was washed successively with 10% aqueous citric acid solution (30 mL), saturated aqueous NaHCO₃ solution (30 mL) and brine (30 mL). The organic layer was dried over anhydrous MgSO₄, and filtered off. The filtrate was concentrated in vacuo. The residue was purified by flash silica-gel column chromatography (gradient elution: AcOEt/hexane=0/1 to 1/0) to give 2-(4-fluorophenyl)-3-[1-(2-methylphenyl)-6-oxo-1,6-dihydropyridazin-3-yl]-4,5-dihydropyrazolo[1,5-a]pyrimidin-6(7H)-one oxime (122 mg, geometrical isomer ratio=1:3) as a brown solid.

Mass ESI (+) 431 (M+1)

¹H-NMR (CDCl₃) δ 2.23 (3H, s), 4.00 (2H, d, J=2.0 Hz), 4.28 (0.6H, d, 2.0 Hz), 4.79 (0.6H, s), 5.04 (2H, s), 5.98 (2H, brs), 6.84 (0.3H, d, J=10.0 Hz), 6.84 (1H, d, J=10.0 Hz), 7.03 (0.3H, d, J=10.0 Hz), 7.04 (1H, d, J=10.0 Hz), 7.16 (0.6H, dd, J=8.5 Hz, J=8.5 Hz), 7.17 (2H, dd, J=8.5 Hz, J=8.5 Hz), 7.32-7.42 (4H, m), 7.51 (2H, dd, J=9.0 Hz, J=5.5 Hz), 7.51 (0.6H, dd, J=9.0 Hz, J=5.5 Hz), 7.82 (1H, s), 7.97 (0.3H, s)

### EXAMPLE 123

To a solution of 6-[(5S)-5-({[tert-butyl(diphenyl)silyl]oxy}methyl)-2-(4-fluorophenyl)-4,5,6,7-tetrahydropyrazolo[1,5-a]pyrimidin-3-yl]-2-(2-methylphenyl)pyridazin-3(2H)-one (450 mg) in THF (4.5 mL) was added a solution of 1 M tetrabutylammonium fluoride in THF (0.67 mL) at rt. After stirring for 30 min, the mixture was partitioned between EtOAc and H₂O. The organic layer was dried over MgSO₄, filtered and concentrated in vacuo. The residue was purified by SiO₂ column chromatography (eluent: 2-5% MeOH in CHCl₃). The obtained oil was crystallized from i-PrOH-Hex to give 6-[(5S)-2-(4-fluorophenyl)-5-(hydroxymethyl)-4,5,6,7-tetrahydropyrazolo[1,5-a]pyrimidin-3-yl]-2-(2-methylphenyl)pyridazin-3(2H)-one (269 mg).

Mass ESI (+) 454 (M+Na)

¹H-NMR (DMSO-d₆) δ 1.75-1.84 (1H, m), 2.05-2.09 (1H, m), 2.11 (3H, s), 3.26-3.33 (1H, m), 3.40 (1H, m), 3.48-3.52 (1H, m), 4.00-4.11 (2H, m), 4.89 (1H, t, 5 Hz), 6.11 (1H, brs), 6.92 (1H, d, J=10 Hz), 7.03 (1H, d, J=10 Hz), 7.26 (2H, t, J=9 Hz), 7.31-7.37 (4H, m), 7.49 (2H, dd, J=9 Hz, 5 Hz)

### EXAMPLE 124

To a solution of 6-[(5S)-5-({[tert-butyl(diphenyl)silyl]oxy}methyl)-2-(4-fluorophenyl)-4,5,6,7-tetrahydropyrazolo[1,5-a]pyrimidin-3-yl]-2-(2-methylphenyl)pyridazin-3(2H)-one (450 mg) in THF (4.5 mL) was added a solution of 1 M tetrabutylammonium fluoride in THF (0.67 mL) at rt. After stirring for 30 min, the mixture was partitioned between EtOAc and H₂O. The organic layer was dried over MgSO₄, filtered and concentrated in vacuo. The residue was purified by SiO₂ column chromatography (eluent: 2-5% MeOH in CHCl₃). The obtained oil was crystallized from i-PrOH-Hex to give 6-[(5S)-2-(4-fluorophenyl)-5-(hydroxymethyl)-4,5,6,7-tetrahydropyrazolo[1,5-a]pyrimidin-3-yl)-2-(2-methylphenyl)pyridazin-3(2H)-one (269 mg).

Mass ESI (+) 454 (M+Na)

¹H-NMR (DMSO-d₆) δ 1.71-1.84 (1H, m), 2.05-2.09 (1H, m), 2.11 (3H, s), 3.26-3.33 (1H, m), 3.40 (1H, m), 3.48-3.52 (1H, m), 3.98-4.14 (2H, m), 4.89 (1H, t, 5 Hz), 6.11 (1H, brs), 6.92 (1H, d, J=10 Hz), 7.03 (1H, d, J=10 Hz), 7.26 (2H, t, J=9 Hz), 7.31-7.37 (4H, m), 7.49 (2H, dd, J=9 Hz, 5 Hz)

### EXAMPLE 125

(i) To a solution of lithium N,N-bistrimethylsilylamide (1.55 mL, 1 M solution in THF) in THF (5 mL) was slowly added a mixture of 6-[2-(4-fluorophenyl)-2-oxoethyl]-2-(2-methylphenyl)pyridazin-3(2H)-one (500 mg) and THF (10 ml) below −60° C. on dry ice-acetone bath, and the whole mixture was stirred at −78° C. for 30 min. To the mixture was added cyanocarbonyloxyethane (154 mg), and the whole was stirred at −78° C. for 3 h and then at room temperature for 6.5 h. The reaction mixture was diluted with saturated aq. NH₄Cl (30 ml) and extracted with AcOEt (50 ml). The organic layer was washed with H₂O and brine (30 ml), dried over MgSO₄ and evaporated. The residue was purified by column chromatography (eluent: CHCl₃-MeOH) to give ethyl 3-(4-fluorophenyl)-2-[1-(2-methylphenyl)-6-oxo-1,6-dihydropyridazin-3-yl)-3-oxopropanoate (254 mg) as a pale yellow oil.

### Mass ESI (−) 393 (M−1)

(ii) A mixture of ethyl 3-(4-fluorophenyl)-2-[1-(2-methylphenyl)-6-oxo-1,6-dihydropyridazin-3-yl)-3-oxopropanoate obtained in above (i) (196 mg), hydrazine monohydrochloride (40.0 mg) and DMF (4 mL) was stirred at room temperature for 5 h. The reaction mixture was diluted with AcOEt (30 ml), and washed with H₂O (20 ml×3) and brine (20 ml). A separated solid in organic layer was collected to give 6-[3-(4-fluorophenyl)-5-hydroxy-1H-pyrazol-4-yl]-2-(2-methylphenyl)pyridazin-3(2H)-one (19.5 mg) as a colorless powder.

Mass ESI (+) 385 (M+Na)

(iii) To a mixture of 6-(3-(4-fluorophenyl)-5-hydroxy-1H-pyrazol-4-yl]-2-(2-methylphenyl)pyridazin-3(2H)-one obtained in above (ii) (100 mg) and DMF (35 mL) were added K₂CO₃ (152 mg) and 1,2-dibromoethane (52.3 mg) at room temperature, and the whole mixture was stirred at 50° C. for 8 h. The mixture was diluted with AcOEt (300 ml). The whole mixture was washed with H₂O (150 ml×3) and brine (50 ml), and the organic layer was dried over MgSO₄ and evaporated. The residue was purified by column chromatography (eluent: CHCl₃-MeOH) to give 6-[6-(4-fluorophenyl)-2,3-dihydropyrazolo[5,1-b][1,3]oxazol-7-yl]-2-(2-methylphenyl)pyridazin-3(2H)-one (45 mg) as a pale yellow amorphous solid.

Mass ESI (+) 411 (M+Na)

¹H-NMR (CDCl₃) δ 2.02 (3H, s), 4.39 (2H, t, J=8 Hz), 5.20 (2H, t, J=8.2 Hz), 7.05 (1H, d, J=9.6 Hz), 7.12-7.19 (2H, m), 7.22-7.38 (5H, m), 7.52-7.59 (2H, m)

### EXAMPLE 126

6-[2-(4-Fluorophenyl)-6,7-dihydro-5H-pyrazolo[5,1-b)[1,3]oxazin-3-yl]-2-(2-methylphenyl)pyridazin-3(2H)-one was obtained according to a similar manner to Example 125.

Mass ESI (+) 425 (M+Na)

¹H-NMR (CDCl₃) δ 2.13 (3H, s), 2.36 (2H, m), 4.26 (2H, t), 4.42 (2H, t), 6.99 (3H, m), 7.30 (5H, m), 7.52 (2H, m)

### EXAMPLE 127

6-[2-(4-Fluorophenyl)-5,6,7,8-tetrahydropyrazolo[5,1-b][1,3]oxazepin-3-yl]-2-(2-methylphenyl)pyridazin-3(2H)-one was obtained according to a similar manner to Example 125.

Mass ESI (+) 439 (M+Na)

¹H-NMR (CDCl₃) δ 1.82-1.90 (2H, m), 1.99-2.08 (5H, m), 4.17-4.29 (4H, m), 7.09-7.21 (4H, m), 7.24-7.36 (3H, m), 7.48-7.56 (3H, m)

### EXAMPLE 128

To a solution of 2-(2-methylphenyl)-6-(2-phenylpyrazolo[1,5-a]pyrazin-3-yl)pyridazin-3(2H)-one (200 mg) in THF (2 mL) and EtOH (1 mL) was added a solution of NaBH₄ in H₂O (0.2 mL) at rt. After 2 h with stirring, the mixture was hearted at 50° C. for 10 min, then quenched by the addition of 1N HCl and adjusted to pH 3. The whole mixture was stirred for 15 min, made alkaline with sat. aq. NaHCO₃ and extracted with EtOAc. The organic layer was dried over Na₂SO₄, filtered and concentrated in vacuo. The residue was purified by column chromatography on SiO₂ (eluent: 5% MeOH in CHCl₃). The obtained oil was treated with 4N HCl, concentrated and triturated with EtOAc to give 2-(2-methylphenyl)-6-(2-phenyl-4,5,6,7-tetrahydropyrazolo[1,5-a]pyrazin-3-yl)pyridazin-3(2H)-one hydrochloride (137 mg) as a powder.

Mass ESI (+) 384 (M+1)

¹H-NMR (DMSO-d₆) δ 2.14 (3H, s), 3.63-3.74 (2H, m), 4.35-4.48 (4H, m), 7.04 (1H, d, J=9.8 Hz), 7.13 (1H, d, J=9.8 Hz), 7.26-7.58 (10H, m), 9.61-9.76 (2H, m).

### EXAMPLE 129

To a suspension of 2-(2-methylphenyl)-6-(2-phenyl-4,5,6,7-tetrahydropyrazolo[1,5-a]pyrazin-3-yl)pyridazin-3(2H)-one hydrochloride (95.0 mg) in CH₂Cl₂ (1.9 mL) were added acetic anhydride (0.032 mL) and N-ethyl-N,N-diisopropylamine (0.118 mL), successively. After stirring for 2 h, the mixture was concentrated in vacuo and partitioned between EtOAc and H₂O. The organic layer was washed with brine, dried over Na₂SO₄, filtered and concentrated. The residue was purified by column chromatography on SiO₂ (eluent: 5% MeOH in CHCl₃) and triturated with diisopropyl ether to give 6-(5-acetyl-2-phenyl-4,5,6,7-tetrahydropyrazolo[1,5-a]pyrazin-3-yl)-2-(2-methylphenyl)pyridazin-3(2H)-one (59 mg) as powder.

Mass ESI (+) 448 (M+Na)
¹H-NMR (DMSO-d₆) δ 2.02-2.21 (6H, m), 3.90-3.99 (2H, m), 4.11-4.32 (2H, m), 4.68-4.82 (2H, m), 7.01 (1H, d, J=9.6 Hz), 7.11-7.17 (1H, m), 7.25-7.56 (9H, m)

### EXAMPLE 130

To a solution of 2-(2-methylphenyl)-6-(2-phenyl-4,5,6,7-tetrahydropyrazolo[1,5-a]pyrazin-3-yl)pyridazin-3(2H)-one hydrochloride (80.0 mg) and acetone (0.046 mL) in CH₂Cl₂ (1.6 mL) was added NaBH(OAc)₃ (88.4 mg) at rt. After 14 h with stirring, the mixture was quenched with 1N HCl (1 mL) and partitioned between EtoAc and sat. aq. NaHCO₃. The organic layer was washed with sat. aq. NaHCO₃ and brine, dried over Na₂SO₄, filtered and concentrated in vacuo. The residue was purified by column chromatography on SiO₂ (eluent: 5% MeOH in CHCl₃). The obtained oil was triturated with diisopropyl ether to give 6-(5-isopropyl-2-phenyl-4,5,6,7-tetrahydropyrazolo[1,5-a]pyrazin-3-yl)-2-(2-methylphenyl)pyridazin-3(2H)-one (67 mg) as powder.

Mass ESI (+) 426 (M+1)

¹H-NMR (DMSO-d₆) δ 1.02 (6H, d, J=6.9 Hz), 2.11 (3H, s), 2.88 (1H, q, J=6.9 Hz), 2.95 (2H, t, J=5.3 Hz), 3.73 (2H, s), 4.14 (2H, t, J=5.3 Hz), 6.99 (1H, d, J=9.9 Hz), 7.16 (1H, d, J=9.9 Hz), 7.31-7.51 (9H, m).

### EXAMPLE 131

To a suspension of NaH (88 mg, 55% in oil) in THF (4 mL) was added a solution of ethyl diethylphosphonoacetate in THF (2 mL) at 0° C., and the solution was stirred at the same temperature for 30 minutes. To the solution was added a solution of 2-(4-fluorophenyl)-3-[1-(2-methylphenyl)-6-oxo-1,6-dihydropyridazin-3-yl]-4,5-dihydropyrazolo[1,5-a]pyrimidin-6(7H)-one (691 mg) in THF (2 mL) at 0° C., and the solution was stirred at the same temperature for 1 hour. To the reaction mixture was added saturated aqueous NH₄Cl solution (5 mL), and the solution was extracted with CH₂Cl₂ (10 mL×2). The extracts were combined, and the solution was washed successively with 10% aqueous citric acid solution (10 mL×2), saturated aqueous NaHCO₃ solution (10 mL) and brine (10 mL). The organic layer was dried over anhydrous MgSO₄, and filtered off. The filtrate was concentrated in vacuo. The residue was purified by flash silica-gel column chromatography (gradient elution: AcOEt/hexane=0/1 to 1/0) to give ethyl {2-(4-fluorophenyl)-3-(1-(2-methylphenyl)-6-oxo-1,6-dihydropyridazin-3-yl]-4,5-dihydropyrazolo[1,5-a]pyrimidin-6-yl}acetate (311 mg) as a pale yellow solid.

Mass ESI (+) 486 (M+1)

¹H-NMR (CDCl₃) δ 1.27 (3H, t, J=7.0 Hz), 2.22 (3H, s), 3.07 (2H, s), 4.16 (2H, q, J=7.0 Hz), 4.14 (2H, s), 5.73 (1H, brs), 6.81 (1H, d, J=10.0 Hz), 6.82 (1H, s), 7.00 (1H, d, J=10.0 Hz), 7.16 (2H, dd, J=8.5 Hz, J=8.5 Hz), 7.30-7.42 (4H, m), 7.50 (2H, dd, J=5.5 Hz, J=8.5 Hz)

### EXAMPLE 132

6-[2-(4-Fluorophenyl)-6,6-dimethyl-4,5,6,7-teterahydropyrazolo[1,5-a]pyrimidin-3-yl]-2-(2-methylphenyl)pyridazin-3(2H)-one was obtained according to a similar manner to Example 60.

Mass ESI (+) 430 (M+1)

¹H-NMR (DMSO-d₆) δ 1.03 (6H, s), 2.09 (3H, s), 2.95 (2H, s), 3.76 (2H, s), 6.09 (1H, br), 6.93 (1H, d, J=10 Hz), 7.11 (1H, d, J=10 Hz), 7.23 (2H, t, J=9 Hz), 7.32-7.38 (4H, m), 7.49 (2H, dd, J=9 Hz, 5 Hz)

The compounds of the present invention are listed in the following tables.

| No. | Structure |
|---|---|
| 1 | 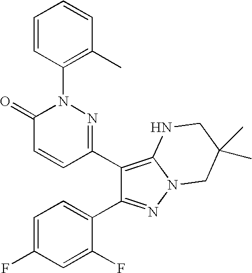 |
| 2 | 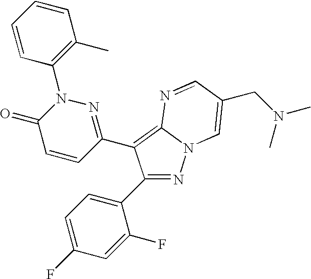 |
| 3 | 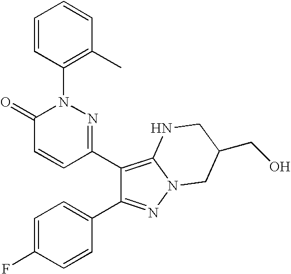 |
| 4 | 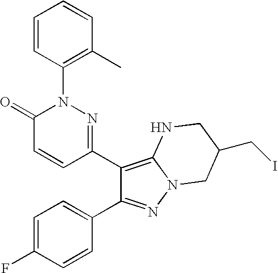 |
| 5 | 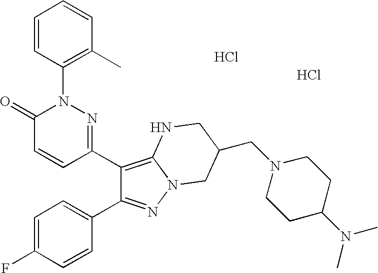 |
| 6 | 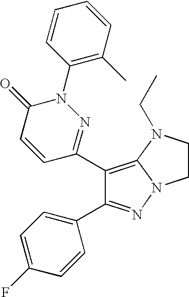 |
| 7 | 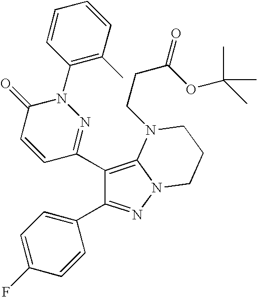 |
| 8 | 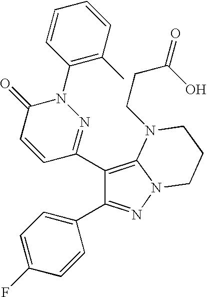 |
| 9 | 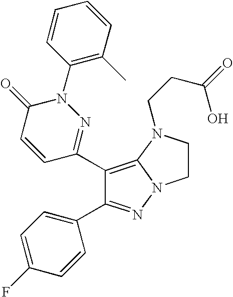 |
| 10 | 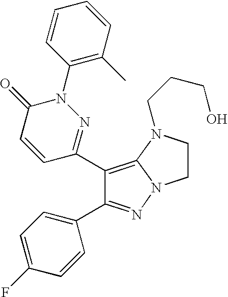 |
| 11 | 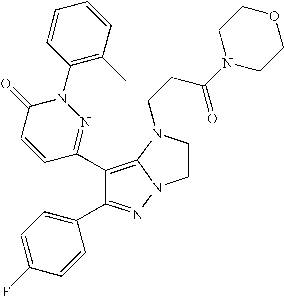 |
| 12 | 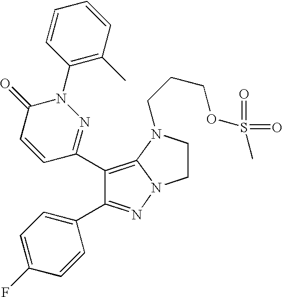 |
| 13 | 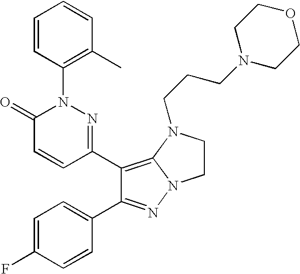 |
| 14 | 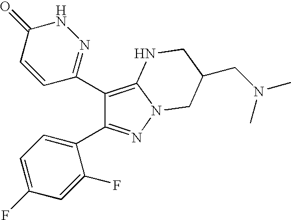 |
| 15 | 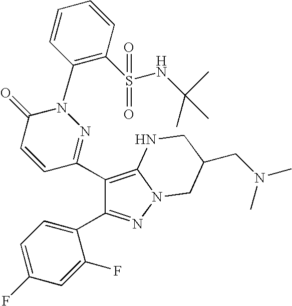 |
| 16 | 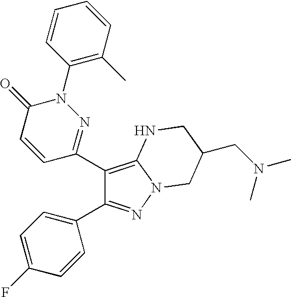 |
| 17 | 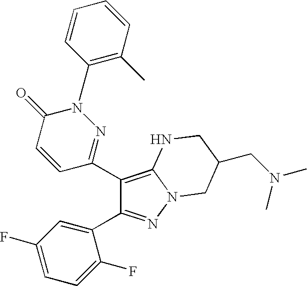 |
| 18 | 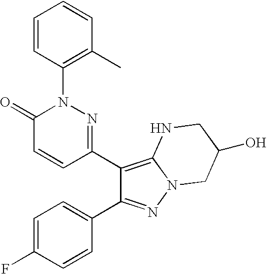 |
| 19 | 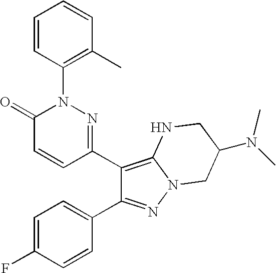 |
| 20 | 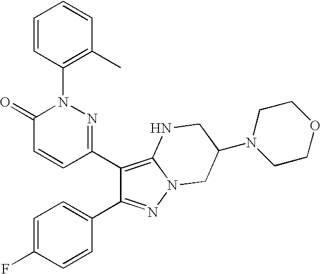 |
| 21 | 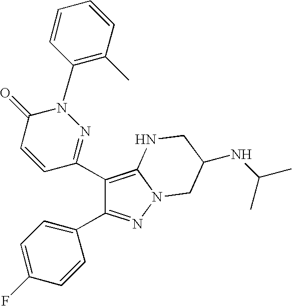 |
| 22 | 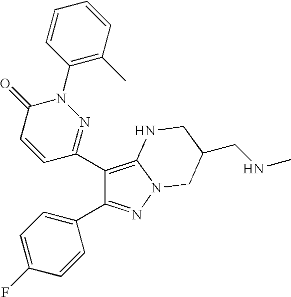 |
| 23 | 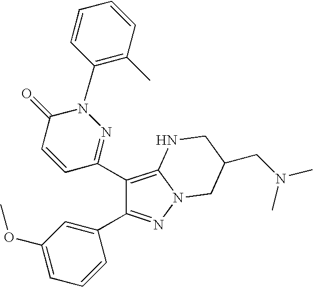 |
| 24 | 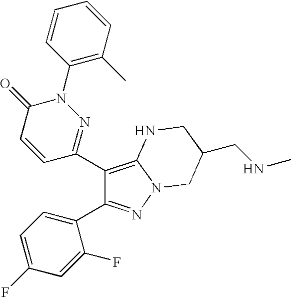 |
| 25 | 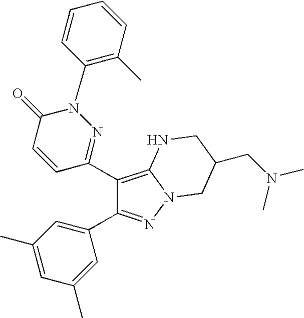 |
| 26 | 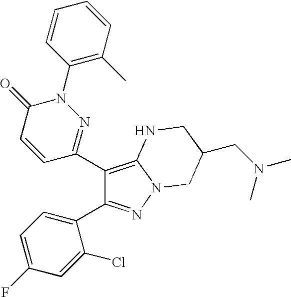 |
| 27 | 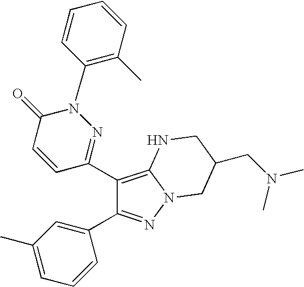 |
| 28 | 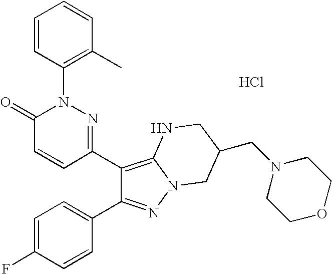 |
| 29 | 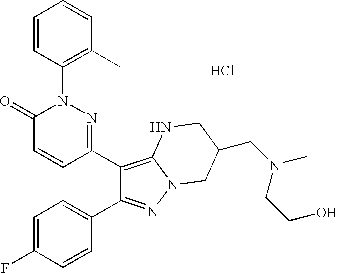 |
| 30 | 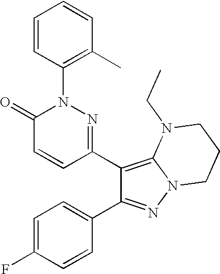 |
| 31 | 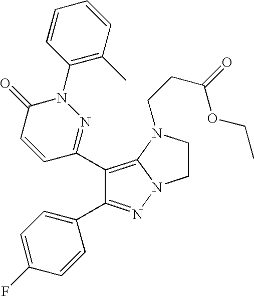 |
| 32 | 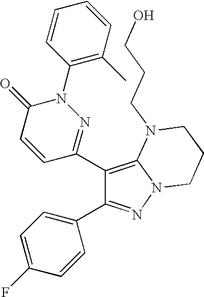 |
| 33 | 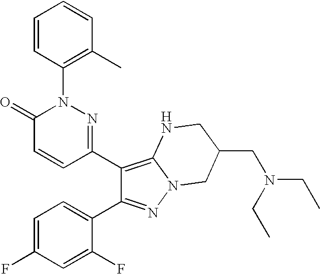 |
| 34 | 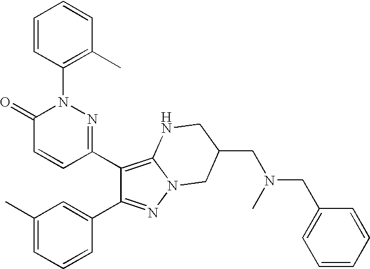 |
| 35 | 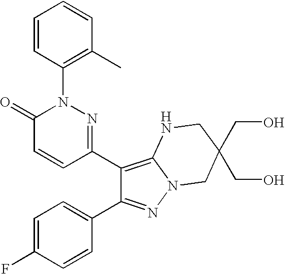 |
| 36 | 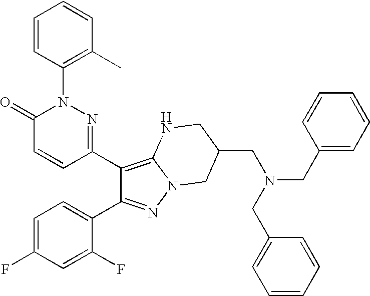 |
| 37 | 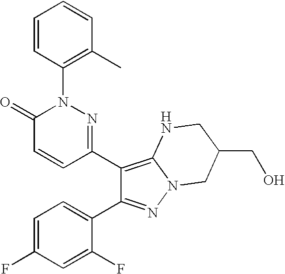 |
| 38 | 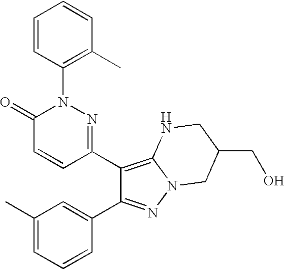 |
| 39 | 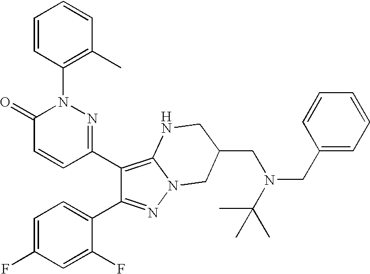 |
| 40 | 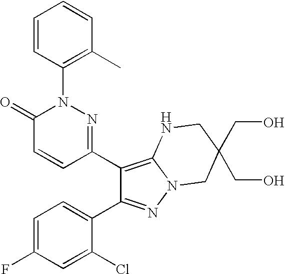 |
| 41 | 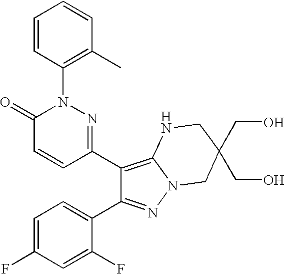 |
| 42 | 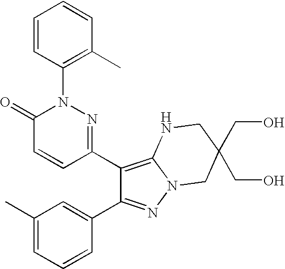 |
| 43 | 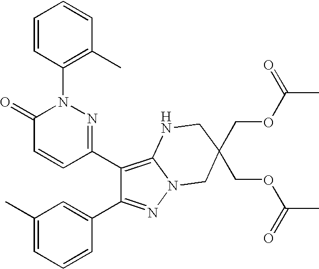 |
| 44 | 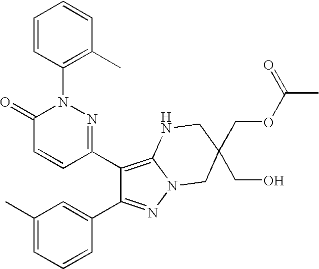 |
| 45 | 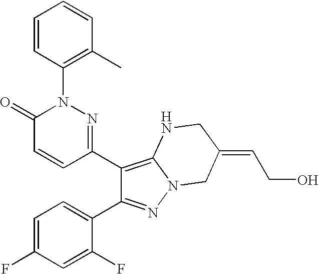 |
| 46 | 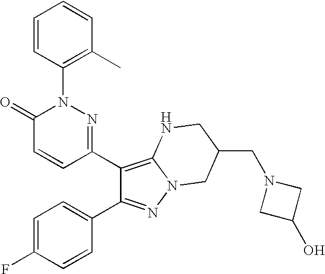 |
| 47 | 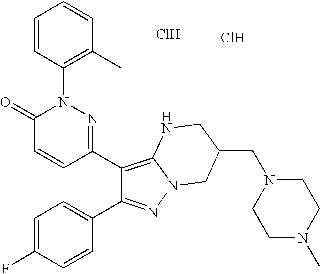 |
| 48 | 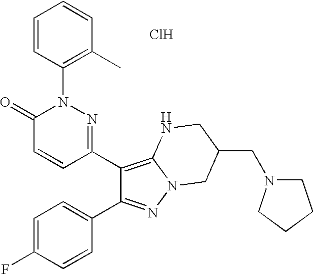 |
| 49 | 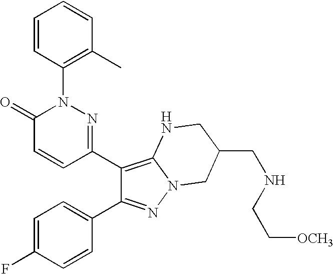 |
| 50 | 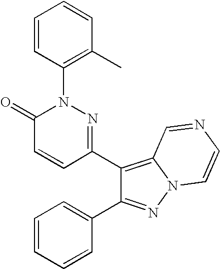 |
| 51 | 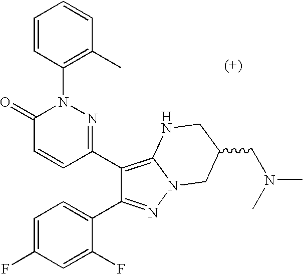 |
| 52 | 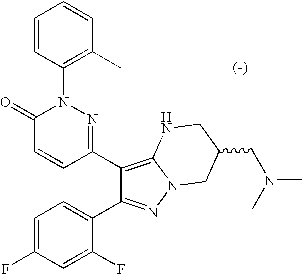 |
| 53 | 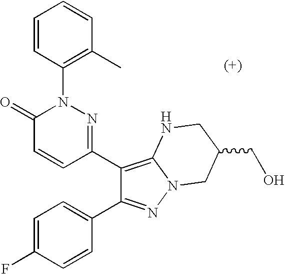 |
| 54 | 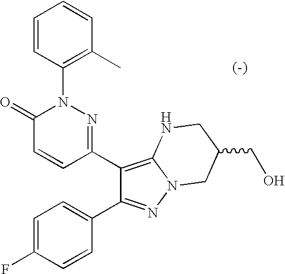 |
| 55 | 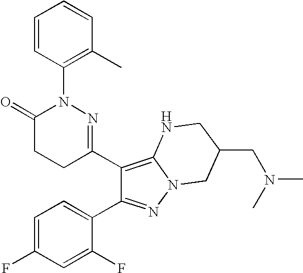 |
| 56 | 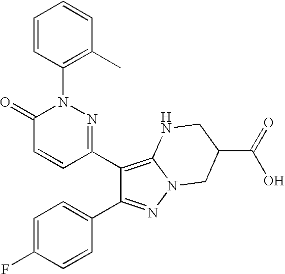 |
| 57 | 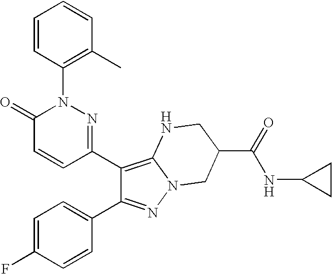 |
| 58 | 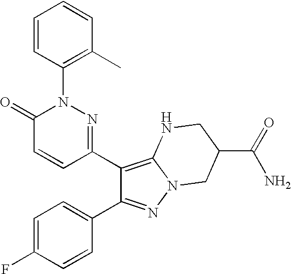 |
| 59 | 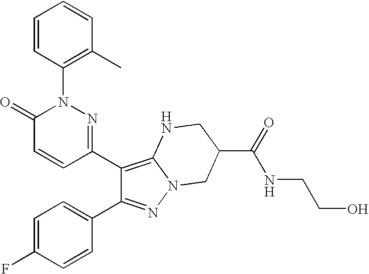 |
| 60 | 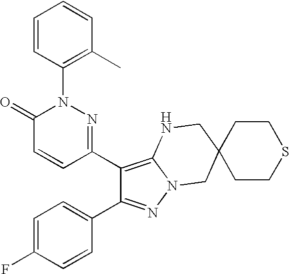 |
| 61 | 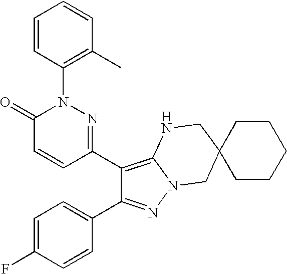 |
| 62 | 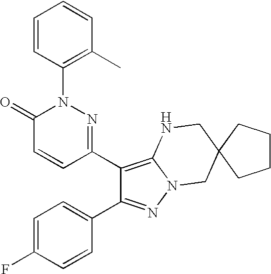 |
| 63 | 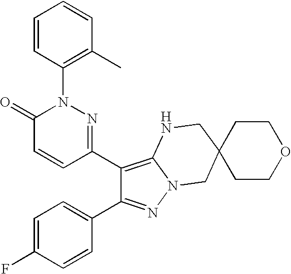 |
| 64 | 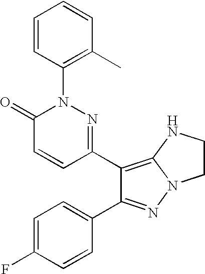 |
| 65 | 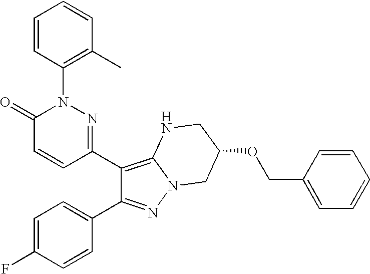 |
| 66 | 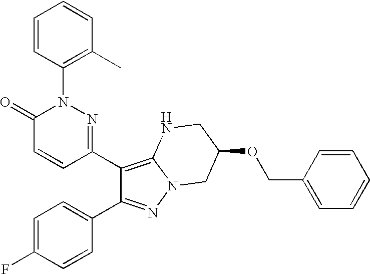 |
| 67 | 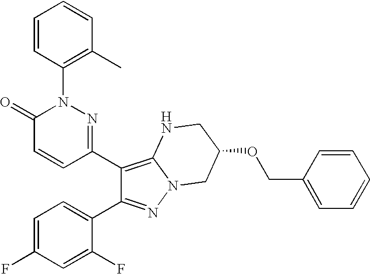 |
| 68 | 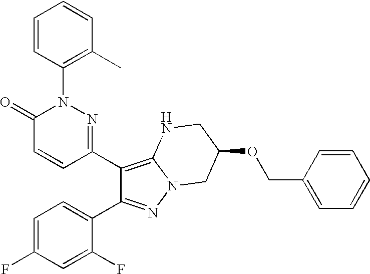 |
| 69 | 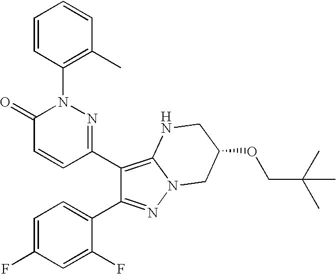 |
| 70 | 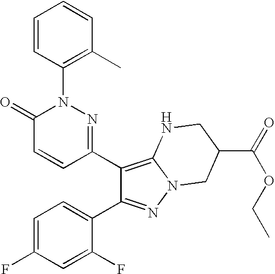 |
| 71 | 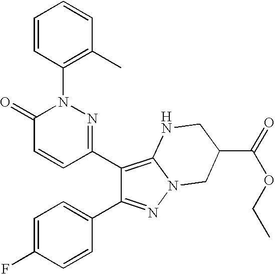 |
| 72 | 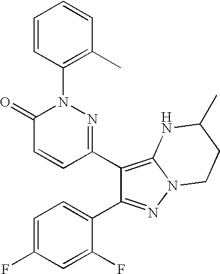 |
| 73 | 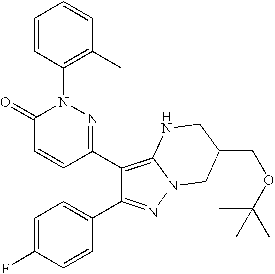 |
| 74 | 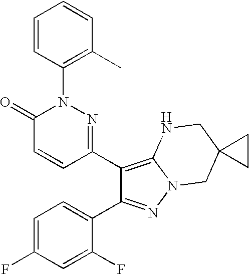 |
| 75 | 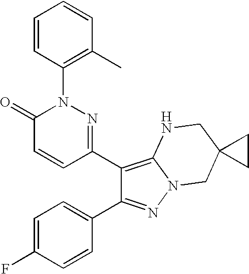 |
| 76 | 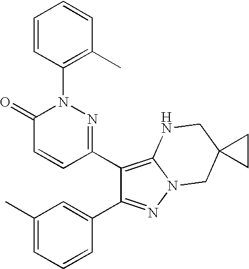 |
| 77 | 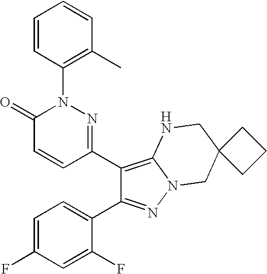 |
| 78 | 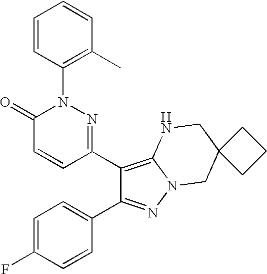 |
| 79 | 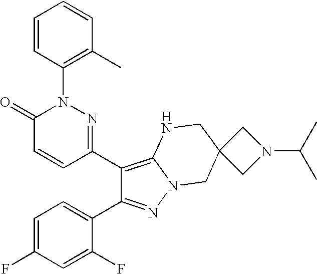 |
| 80 | 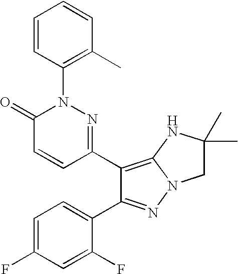 |
| 81 | 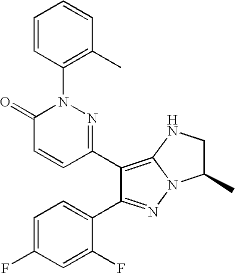 |
| 82 | 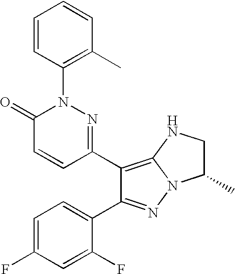 |
| 83 | 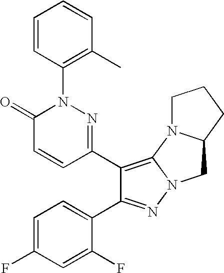 |
| 84 | 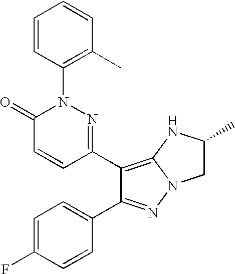 |
| 85 | 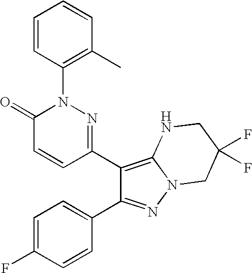 |
| 86 | 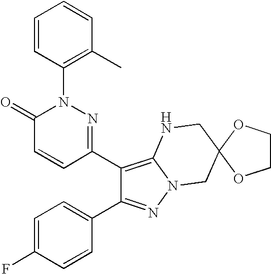 |
| 87 | 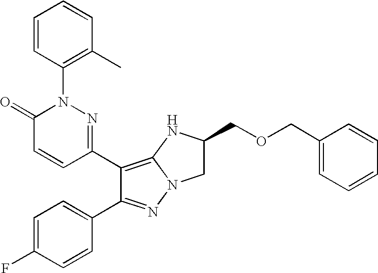 |
| 88 | 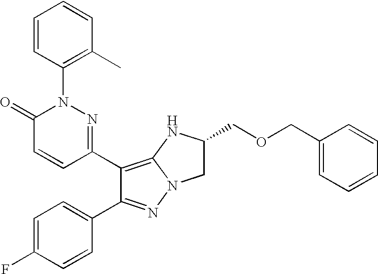 |
| 89 | 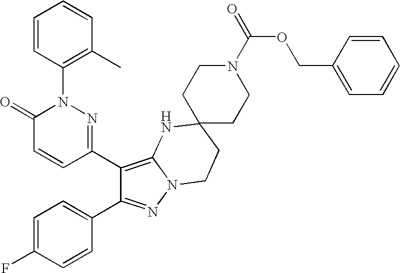 |
| 90 | 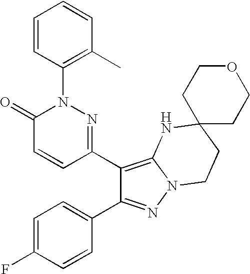 |
| 91 | 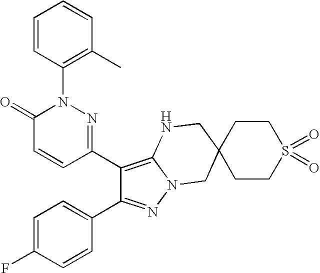 |
| 92 | 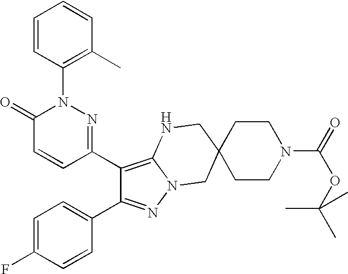 |
| 93 | 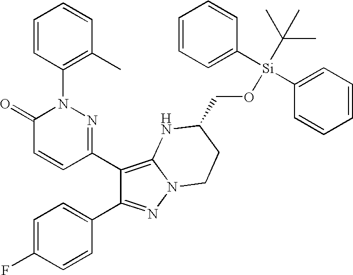 |
| 94 | 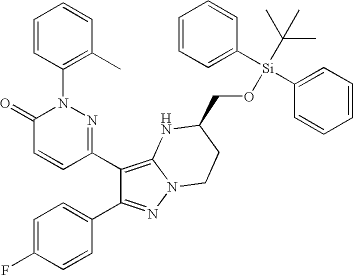 |
| 95 | 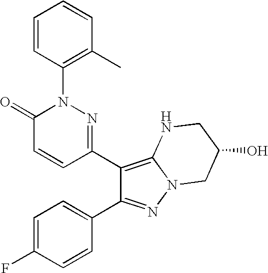 |
| 96 | 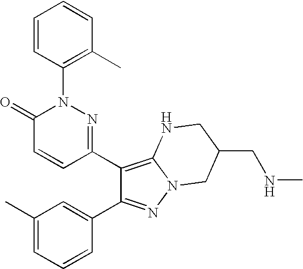 |
| 97 | 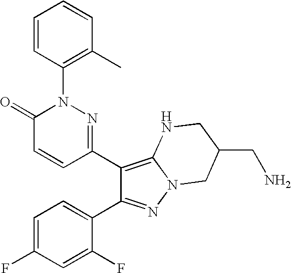 |
| 98 | 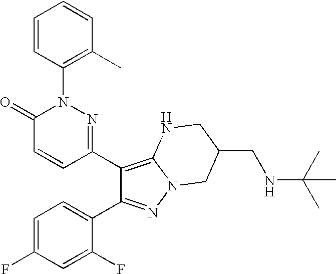 |
| 99 | 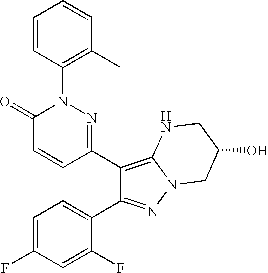 |
| 100 | 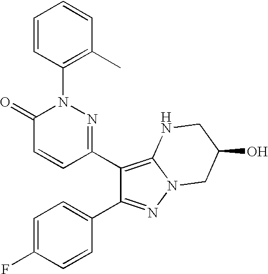 |
| 101 | 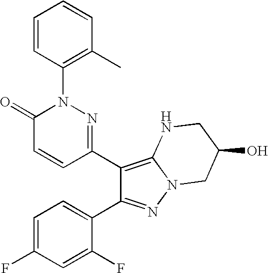 |
| 102 | 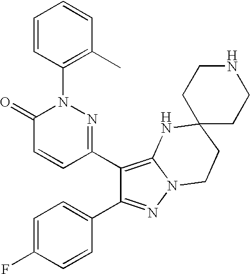 |
| 103 | 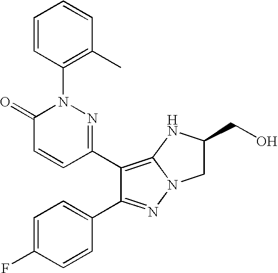 |
| 104 | 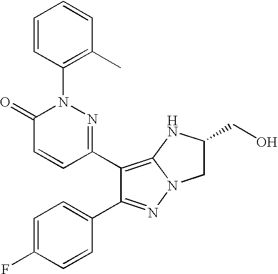 |
| 105 | 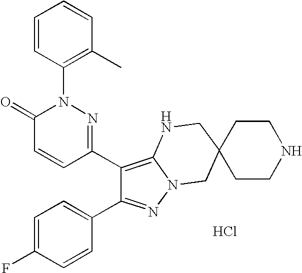 |
| 106 | 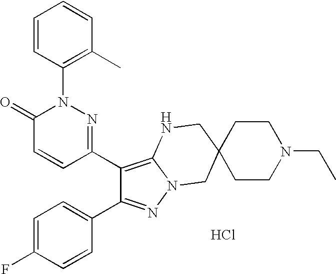 |
| 107 | 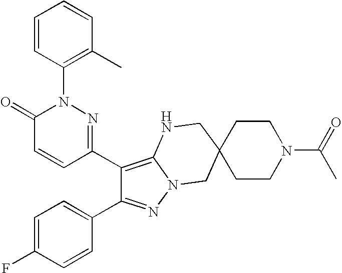 |
| 108 | 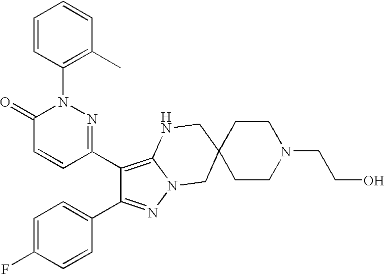 |
| 109 | 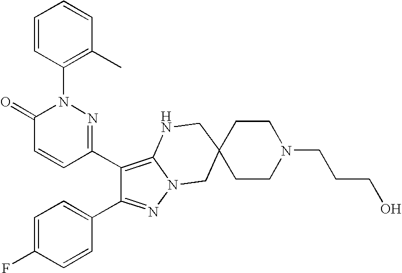 |
| 110 | 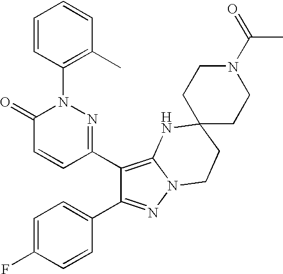 |
| 111 | 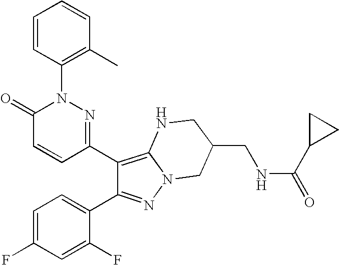 |
| 112 | 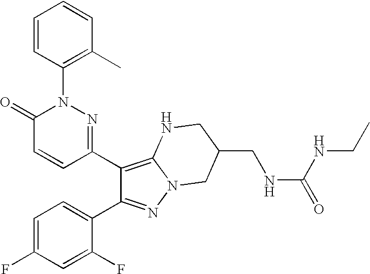 |
| 113 | 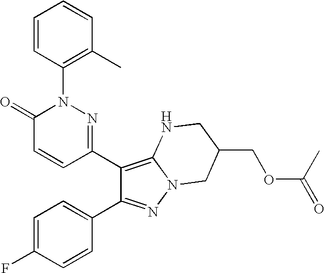 |
| 114 | 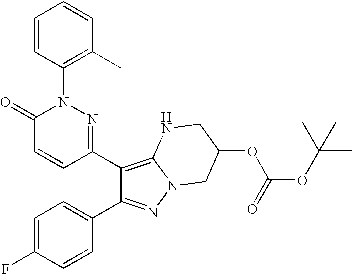 |
| 115 | 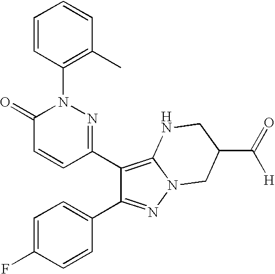 |
| 116 | 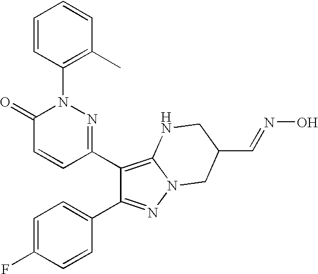 |
| 117 | 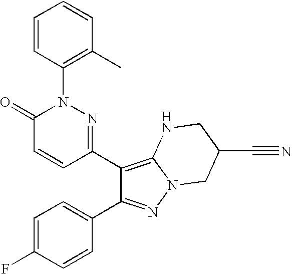 |
| 118 | 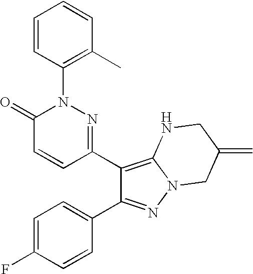 |
| 119 | 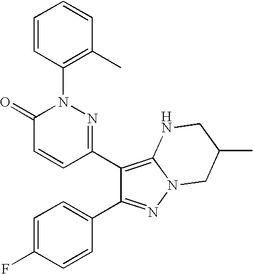 |
| 120 | 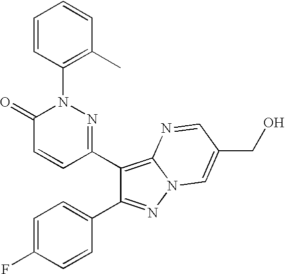 |
| 121 | 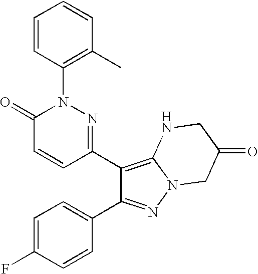 |
| 122 | 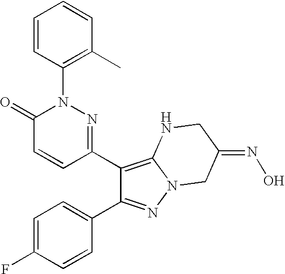 |
| 123 | 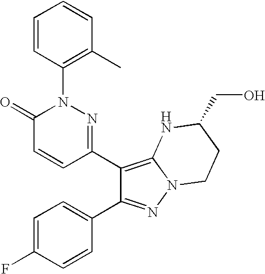 |
| 124 | 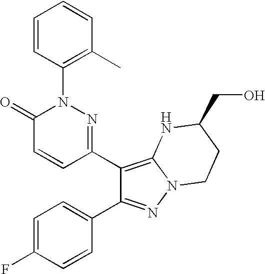 |
| 125 | 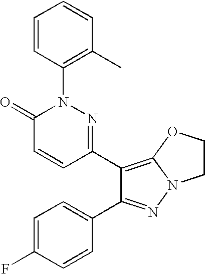 |
| 126 | 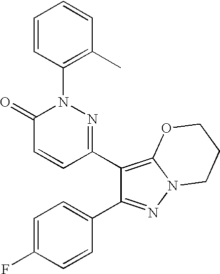 |
| 127 | 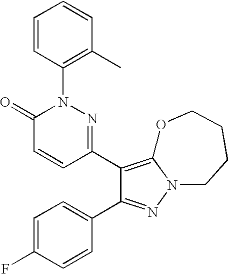 |
| 128 | 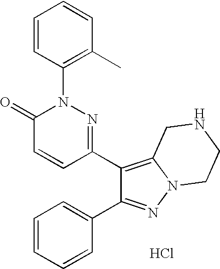 |
| 129 | 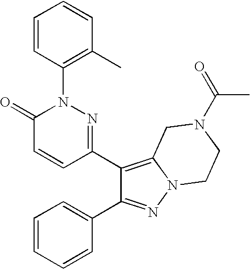 |
| 130 | 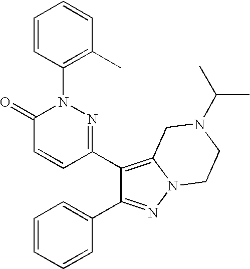 |
| 131 | 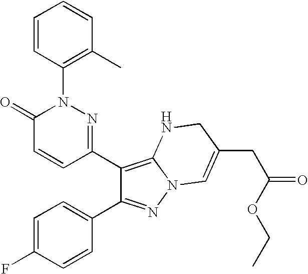 |
| 132 | 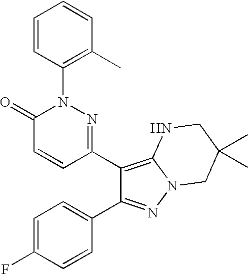 |

### INDUSTRIAL APPLICABILITY

As mentioned above, the present invention can provide a novel pyridazinone derivative compound and a pharmaceutically acceptable salt thereof, and a pharmaceutical composition comprising said compound as an active ingredient and a pharmaceutically acceptable salt thereof. The pyridazinone derivative compound is useful as an active ingredient of a therapeutic or prophylactic agent for various diseases such as pain, rheumatoid arthritis, other conditions associated with inflammation, Crohn's disease, inflammatory bowel disease, psoriasis, etc.

This application is based on the patent application No. 60/712,825, which was filed in the United States on Sep. 1, 2005, and the contents of which are incorporated hereinto by reference.

1. A pyridazinone derivative compound shown by the following formula (I): 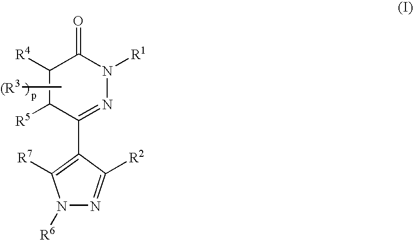
wherein
R¹ is selected from the group consisting of hydrogen, substituted or unsubstituted lower alkyl and substituted or unsubstituted aryl;
R² is selected from the group consisting of substituted or unsubstituted aryl and substituted or unsubstituted heteroaryl;
R³ is lower alkyl;
P is 0, 1 or 2; and
R⁴ and R⁵ are each hydrogen or taken together to form a bond;
R⁶ and R⁷ are taken together to form a group of the formula: 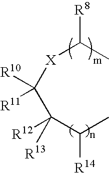
wherein
R⁸ is hydrogen,
X is oxygen or N—R⁹, in which R⁹ is hydrogen, substituted or unsubstituted lower alkanoyl or substituted or unsubstituted lower alkyl; or
R⁸ and R⁹ may be taken together to form a bond;
m and n are each 0, 1 or 2;
R¹⁰ and R¹² are each selected from the group consisting of hydrogen, halogen, hydroxy, formyl, cyano, substituted or unsubstituted lower alkyl, substituted or unsubstituted amino, substituted or unsubstituted lower alkoxy, saturated cyclic amino, substituted or unsubstituted carbamoyl, carboxy, substituted or unsubstituted lower alkoxycarbonyl and substituted or unsubstituted acyloxy;
R¹¹, R¹³ and R¹⁴ are each selected from the group consisting of hydrogen, halogen, substituted or unsubstituted lower alkyl, carboxy and substituted or unsubstituted lower alkoxycarbonyl;
R¹⁰ and R¹¹ or R¹² and R¹³ may be taken together to form oxo, hydroxyimino, substituted or unsubstituted lower alkylene in which one or more carbon(s) may be replaced by hetero atom(s), or substituted or unsubstituted lower alkylidene;
R⁹ and R¹⁰ may be taken together to form lower alkylene or a bond;
R¹¹ and R¹² or R¹³ and R¹⁴ may be taken together to form a bond;
provided that when n=1 and R¹⁰, R¹¹, R¹², R¹³ and R¹⁴ are simultaneously hydrogen, R⁹ is substituted or unsubstituted lower alkyl or substituted or unsubstituted lower alkanoyl, or a pharmaceutically acceptable salt thereof.

2. The pyridazinone derivative compound of claim 1, wherein
R¹ is hydrogen or substituted or unsubstituted aryl;
R² is substituted or unsubstituted aryl;
p is 0;
R⁴ and R⁵ are each hydrogen or taken together to form a bond; and
R⁶ and R^{v} are taken together to form a group of the formula: 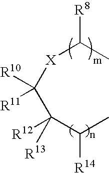
wherein
R⁸ is hydrogen;
X is oxygen or N—R⁹, in which R⁹ is hydrogen, substituted or unsubstituted lower alkanoyl or substituted or unsubstituted lower alkyl; or
R⁸ and R⁹ may be taken together to form a bond;
m and n are each 0, 1 or 2;
R¹⁰ and R¹² are each selected from the group consisting of hydrogen, halogen, hydroxy, formyl, cyano, substituted or unsubstituted lower alkyl, substituted or unsubstituted amino, substituted or unsubstituted lower alkoxy, saturated cyclic amino, substituted or unsubstituted carbamoyl, carboxy substituted or unsubstituted lower alkoxycarbonyl and substituted or unsubstituted acyloxy;
R¹¹, R¹³ and R¹⁴ are each selected from the group consisting of hydrogen, halogen and substituted or unsubstituted lower alkyl;
R¹⁰ and R¹¹ or R¹² and R¹³ may be taken together to form oxo, hydroxyimino, substituted or unsubstituted lower alkylene in which one or more carbon(s) may be replaced by hetero atom(s), or substituted or unsubstituted lower alkylidene;
R⁹ and R¹⁰ may be taken together to form lower alkylene or a bond;
R¹¹ and R¹² or R¹³ and R¹⁴ may be taken together to form a bond,
provided that when n=1 and R¹⁰), R¹¹, R¹², R¹³ and R¹⁴ are simultaneously hydrogen, R⁹ is substituted or unsubstituted lower alkyl or substituted or unsubstituted lower alkanoyl,
or a pharmaceutically acceptable salt thereof.

3. The pyridazinone derivative compound of claim 2, wherein
R¹ is hydrogen or (C₆₋₁₄)aryl optionally substituted by (C₁₋₆)alkyl or (C₁₋₆) alkylaminosulfonyl;
R² is (C₆₋₁₄)aryl optionally substituted by 1 to 3 substituent(s) selected from halogen, (C₁₋₆)alkyl and (C₁₋₆)alkoxy;
p is 0;
R⁴ and R⁵ are each hydrogen or taken together to form a bond; and
R⁶ and R⁷ are taken together to form a group of the formula: 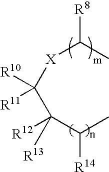
wherein
R⁸ is hydrogen;
X is oxygen or N—R⁹, in which R⁹ is hydrogen, (C₁₋₆)alkyl optionally substituted by carboxy, hydroxy, (C₁₋₆)alkoxycarbonyl, morpholino, morpholinocarbonyl or (C₁₋₆)alkylsulfonyloxy, or (C₂₋₇) alkanoyl; or
R⁸ and R⁹ are taken together to form a bond;
m and n are each 0, 1 or 2;
R¹⁰ is hydrogen, or (C₁₋₆)alkyl optionally substituted by (C₆₋₁₄) aryl(C₁₋₆) alkoxy, di(C₆₋₁₄)aryl(C₁₋₆)alkylsilyloxy or hydroxy;
R¹¹ is hydrogen or (C₁₋₆)alkyl;
R¹² is selected from the group consisting of hydrogen;
halogen;
hydroxy;
carboxy;
formyl;
cyano;
(C₁₋₆)alkyl optionally substituted by hydroxy, hydroxyimino, halogen, (C₁₋₆) alkoxy, (C₁₋₇)alkanoyloxy, amino, mono- or di-(C₁₋₆)alkylamino (wherein one or both of said (C₁₋₆)alkyl is (are) optionally substituted by hydroxy, (C₆₋₁₄) aryl or (C₃₋₆) cycloalkyl-carbonyl), (C₁₋₆)alkylureido, morpholino, or 4- to 6-membered cyclic amino optionally substituted by hydroxy, (C₂₋₆)alkyl or di(C₁₋₆)alkylamino;
mono- or di-(C₁₋₆)alkylamino;
4- to 6-membered cyclic amino;
C₁₋₆ alkoxy optionally substituted by (C₆₋₁₄) aryl;
carbamoyl optionally substituted by (C₃₋₆)cycloalkyl or hydroxy(C₁₋₆)alkyl;
(C₁₋₆)alkoxy-carbonyl; and
(C₁₋₆)alkoxy-carbonyloxy;
R¹³ is hydrogen, or (C₁₋₆)alkyl optionally substituted by hydroxy or (C₁₋₇)alkanoyloxy;
R¹⁴ is hydrogen;
R¹⁰ and R¹¹ may be taken together to form (C₂₋₆)alkylene in which one or more carbon atom(s) may be replaced with heteroatom(s), which is optionally substituted by (C₆₋₁₄)aryl(C₁₋₆) alkoxycarbonyl or (C₁₋₇)alkanoyl;
R¹² and R¹³ may be taken together to form
C₂₋₆ alkylene in which one or more carbon atom(s) may be replaced with heteroatom(s) which is optionally substituted by (C₁₋₆)alkyl optionally substituted by hydroxy, or (C₁₋₇)alkanoyl optionally substituted by C₁₋₆ alkoxy;
(C₁₋₆)alkylidene optionally substituted by hydroxy;
oxo; or
hydroxyimino;
R⁹ and R¹⁰ may be taken together to form (C₂₋₆)alkylene or a bond;
R¹¹ and R¹³ may be taken together to form a bond; or
R¹³ and R¹⁴ may be taken together to form a bond;
provided that when n=1 and R¹⁰, R¹¹, R¹², R¹³ and R¹⁴ are simultaneously hydrogen, R⁹ is substituted or unsubstituted lower alkyl or substituted or unsubstituted lower alkanoyl;
or a pharmaceutically acceptable salt thereof.

4. The compound of claim 1, wherein
R¹ is selected from the group consisting of hydrogen, substituted or unsubstituted lower alkyl and substituted or unsubstituted aryl;
R² is selected from the group consisting of substituted or unsubstituted aryl and substituted or unsubstituted thienyl;
R³ is lower alkyl;
p is 0, 1 or 2;
R⁴ and R⁵ are taken together to form a bond; and
R⁶ and R⁷ are taken together to form a group of the formula: 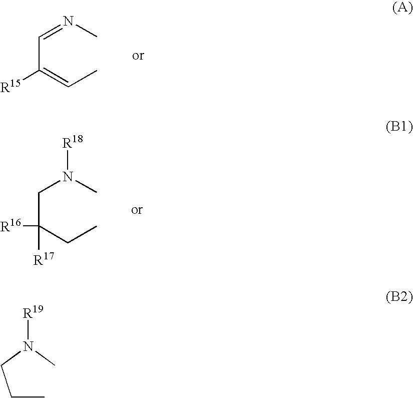
wherein
R¹⁵ is selected from the group consisting of hydroxy, substituted or unsubstituted lower alkyl, substituted or unsubstituted amino, substituted or unsubstituted lower alkoxy, saturated cyclic amino, substituted or unsubstituted carbamoyl, carboxy and substituted or unsubstituted lower alkoxycarbonyl;
R¹⁶ is selected from the group consisting of hydrogen, halogen, hydroxy, substituted or unsubstituted lower alkyl, substituted or unsubstituted amino, saturated cyclic amino, substituted or unsubstituted lower alkoxy, substituted or unsubstituted carbamoyl, carboxy and substituted or unsubstituted lower alkoxycarbonyl;
R¹⁷ is selected from the group consisting of hydrogen, halogen and substituted or unsubstituted lower alkyl; or
R¹⁶ and R¹⁷ are taken together to form lower alkylene or lower alkylidene;
R¹⁸ is hydrogen or substituted or unsubstituted lower alkyl, provided that when both R¹⁶ and R¹⁷ are simultaneously hydrogen, R¹⁸ is substituted or unsubstituted lower alkyl; and
R¹⁹ is hydrogen or substituted or unsubstituted lower alkyl,
or a pharmaceutically acceptable salt thereof.

5. The compound of claim 4, wherein
R¹ is hydrogen or substituted or unsubstituted aryl;
R² is substituted or unsubstituted aryl;
p is 0;
R⁴ and R⁵ are taken together to form a bond; and
R⁶ and R⁷ are taken together to form a group of the formula: 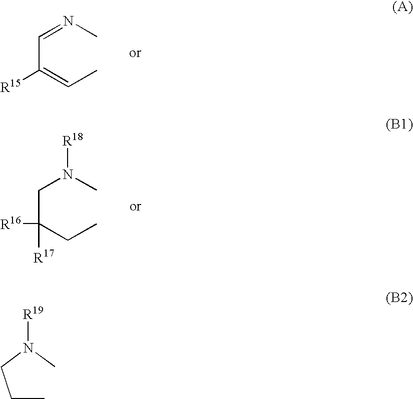
wherein
R¹⁵ is substituted or unsubstituted lower alkyl;
R¹⁶ is selected from the group consisting of hydrogen, hydroxy, substituted or unsubstituted lower alkyl, substituted or unsubstituted amino and saturated cyclic amino;
R¹⁷ is hydrogen;
R¹⁸ is hydrogen or substituted or unsubstituted lower alkyl; and
R¹⁹ is hydrogen or substituted or unsubstituted lower alkyl,
or a pharmaceutically acceptable salt thereof.

6. The compound of claim 5, wherein
R¹ is selected from the group consisting of hydrogen and (C₆₋₁₄)aryl optionally substituted by (C₁₋₆) alkyl or (C₁₋₆)alkylaminosulfonyl;
R² is (C₆₋₁₄)aryl optionally substituted by 1 to 3 substituent(s) selected from halogen, (C₁₋₆)alkyl and (C₁₋₆) alkoxy;
p is 0;
R⁴ and R⁵ are taken together to form a bond; and
R⁶ and R⁷ are taken together to form a group of the formula: 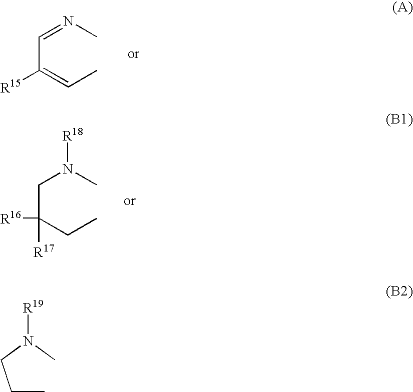
wherein
R¹⁵ is mono- or di-(C₁₋₆)alkylamino-(C₁₋₆)alkyl or hydroxy(C₁₋₆) alkyl;
R¹⁶ is selected from the group consisting of hydrogen;
hydroxy;
C₁₋₆ alkyl optionally substituted by hydroxy, halogen, methylamino, dimethylamino, (2-hydroxyethyl)methylamino, morpholino or 4-(dimethylamino)-1-piperidinyl;
mono- or di-(C₁₋₆)alkylamino; and
piperidino;
R¹⁷ is hydrogen;
R¹⁸ is hydrogen or (C₁₋₆)alkyl optionally substituted by (C₁₋₆)alkoxycarbonyl, carboxy or hydroxy; and
R¹⁹ is (C₁₋₆)alkyl optionally substituted by carboxy, hydroxy, (C₁₋₆)alkoxycarbonyl, morpholino, morpholinocarbonyl or (C₁₋₆)alkylsulfonyloxy,
or a pharmaceutically acceptable salt thereof.

7. A pharmaceutical composition comprising the compound of claim 1 or a pharmaceutically acceptable salt thereof in admixture with a pharmaceutically acceptable carrier.

8. The pharmaceutical composition of claim 7, which is for the prevention or the treatment of a disease selected from the group consisting of pain, rheumatoid arthritis, other conditions associated with inflammation, Crohn's disease, inflammatory bowel disease and psoriasis.

9. A method for preventing or treating a disease selected from the group consisting of pain, rheumatoid arthritis, other conditions associated with inflammation, Crohn's disease, inflammatory bowel disease and psoriasis, which comprises administering an effective amount of the compound of claim 1 or a pharmaceutically acceptable salt thereof to a mammal in need thereof.

10. Use of the compound of claim 1 or a pharmaceutically acceptable salt thereof for the production of a pharmaceutical composition for the prevention or the treatment of a disease selected from the group consisting of pain, rheumatoid arthritis, other conditions associated with inflammation, Crohn's disease, inflammatory bowel disease and psoriasis.
